(19) United States
(12) Patent Application Publication
(10) Pub. No.: US 2004/0156826 A1
(43) Pub. Date: Aug. 12, 2004

(54) Treatment of patients with multiple sclerosis based on gene expression changes in central nervous system tissues

(76) Inventors: Fernando Dangond, Newton, MA (US); Daehee Hwang, Seattle, WA (US); Steven R. Gullans, Natick, MA (US)

Correspondence Address: FULBRIGHT & JAWORSKI L.L.P., SUITE 2400, 600 CONGRESS AVENUE, AUSTIN, TX, 78701-3271, US

(21) Appl. No.: 10/670,766

(22) Filed: Sep. 25, 2003

Related U.S. Application Data
(60) Provisional application No. 60/414,219, filed on Sep. 27, 2002.

(51) Int. Cl. A61K 48/00; A61K 38/21

(52) U.S. Cl. 424/93.2; 514/12; 424/85.6

(57) ABSTRACT

The present invention identifies a number of gene markers whose expression is altered in multiple sclerosis (MS). These markers can be used to diagnose or predict MS in subjects, and can be used in the monitoring of therapies. In addition, these genes identify therapeutic targets, the modification of which may prevent MS development or progression.

The present application claims benefit of priority U.S. Provisional Serial No. 60/414,219, filed Sep. 27, 2002, the entire contents of which are hereby incorporated by reference.

### BACKGROUND OF THE INVENTION

1. Field of the Invention

The present invention relates generally to the fields of molecular biology, genomics, immunology and neurobiology. More particularly, it concerns the identification of specific genes that are dysregulated in patients afflicted with multiple sclerosis (MS), and the use of these genes as targets for MS therapies.

2. Description of Related Art

Multiple sclerosis (MS) continues to be a serious health problem that afflicts hundreds of thousands each year in the US alone, and millions worldwide. One of the difficult aspects of dealing with MS is identifying patients early in the course of the disease. This is difficult not only because of the lack of a definitive biological test for MS, but because the symptoms may overlap with those of numerous other diseases.

The concordance rate of multiple sclerosis among monozygotic twins is 20-40%, while the risk of a non-twin sibling of an MS patient of developing MS is 2-4%. These facts highly suggest the presence of polygenic susceptibility (nonmendelian inheritance). Although no single gene is associated with all types of MS, several reports have revealed that some genes are associated with MS in certain populations. The well known HLA association with MS has been demonstrated in populations of northern European ancestry. In the Finnish population an association with the myelin basic protein gene has been reported (Tienari et al., 1992). In an European MS patient population, an association with a T cell differentiation-related antigen, CD45, has been demonstrated (Jacobsen et al., 2000).

Since the disease is polymorphic (i.e., not inherited in a classical mendelian pattern but clearly multiple genes are involved in leading to predisposition), recent genomic approaches have been implemented to elucidate multiple genes simultaneously that may be associated with the disease. A recent publication by Lock et al. (2002) demonstrates how gene expression profiling using DNA microarrays to examine MS brain tissues can help identify multiple single genes that are associated with the disease, and may therefore serve as targets of treatment. By altering the function of the product of some of these genes in the animal model of MS, experimental autoimmune encephalomyelitis (EAE), these authors confirmed that some genes found to be altered by DNA microarray screening indeed had an impact on the severity of the disease.

Another approach to identify potential single gene associations is to examine polymorphic gene variants or single nucleotide polymorphisms (SNPs) of candidate genes, or screen the entire genome to establish the SNPs that are associated with the disease. Multiple polymorphisms have been associated with MS, as follows: (a) polymorphisms associated with MS disease susceptibility found in the following genes: SCA2 (Chataway et al., 1999), interferon α (Miterski et al., 1999), estrogen receptor (Niino et al., 2000), plasminogen activator inhibitor 1 (Luomala et al., 2000), tumor necrosis factor α (Fernandez-Arquero et al., 1999; Lucotte et al., 2000), monocyte chemotactic protein 3 (Fiten et al., 1999), vitamin D receptor (Fukazawa et al., 1999), CTLA4 (Fukazawa et al., 1999), γ aminobutyric acid (Gade-Andavolu et al., 1998); (b) polymorphisms associated with disease severity found in the following genes: interleukin 6 (Vandenbroeck et al., 2000), IgG Fc receptor (FcγR) (Myhr et al., 1999), glutathione-S-transferase (Mann et al., 2000); (c) polymorphisms associated with age of onset of MS found in the following genes: interleukin 4 (Vandenbroeck et al., 1997) and chemokine receptor CCR5 (Barcellos et al., 2000); and (d) polymorphism associated with remyelination capacity: apolipoprotein E (Carlin et al., 2000). Other gene polymorphisms that have been associated with MS include intercellular adhesion molecule 1 (ICAM-1) (Mycko et al., 1998), the pro-inflammatory gene lymphotoxin (Mycko et al., 1998) and immunoglobulin heavy chain gene polymorphisms (Hashimoto et al., 1993; Walter et al., 1991).

Despite these individual associations, there has yet to be put forth a cohesive set of genes that provide clearly relevant targets for genetic based therapies.

### SUMMARY OF THE INVENTION

Thus, in accordance with the present invention, there is provided a method for treating or preventing multiple sclerosis (MS) comprising administering to a subject with MS a composition that causes an increase in the level of a gene product selected from the group consisting of those genes indicated by a minus (−) sign in Tables 1-15, except those indicated by asterisk(s). In still yet a further embodiment, there is provided a method for treating or preventing multiple sclerosis (MS) comprising administering to a subject with MS a composition that causes a decrease in the level of a gene product selected from the group consisting of those genes indicated by a plus (+) sign in Tables 1-15, except those indicated by asterisk(s). Further, genes from Table 16, 17, or 18 are lists of genes previously reported to be associated with MS central nervous system tissues by Lock et al. (2002), Chabas et al. (2001), and Whitney et al. (1999), respectively, and are indicated by asterisks in Tables 1-15 as also found by the presented inventors to be dysregulated in MS spinal cords, may be used as targets in combination with one or more of the genes from Tables 1-15.

The inventors also found, quite strikingly, that the CD18 (probe set X64072, also represented by accession number M15395) subunit of lymphocyte function antigen-1 (LFA-1) and of CR3 and CR4 complement, is highly upregulated in all MS samples (including samples with minimal or no inflammation by histological criteria). CD18 plays a role in immune cell activation, cell-cell contacts and as a mediator of phagocytosis. Bowen et al. (1998) reported a Phase I study using humanized monoclonal antibodies against CD18 protein in MS patients. In addition, Yusuf-Makagiansar et al. (2002) proposed the use of antibodies, peptides and small molecules against CD18 protein to treat autoimmune diseases and inflammation. The present inventors intention, based on striking findings of CD18 mRNA elevation in MS spinal cords, is to target the expression of CD18 mRNA, not protein, in MS central nervous system tissues using technologies such as antisense constructs, RNA interference and other methods described further below.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 Kernel density estimate based on five ratios.

FIGS. 2A & 2B Kernel density estimate and histogram of ratios with an adjusted bandwidth.

### DESCRIPTION OF ILLUSTRATIVE EMBODIMENTS

In autoimmune diseases, activated T and B cells are hypothesized to clonally expand (i.e., proliferate into multiple daughter cells) and lead to tissue destruction, via infiltration of target tissues with direct cytotoxicity and/or release of harming soluble factors or antibodies. Macrophages are also important mediators of tissue damage. MS is widely considered an autoimmune disease, but there is significant controversy about the key molecules that participate in such process. It also is a heterogeneous disease, and within a single patient, one finds different degrees (and localization in anatomical regions) of demyelination, inflammation and degeneration. The inventors thus examined post-mortem spinal cords via histopathology techniques to determine what type of multiple sclerosis lesions they were working with. Using DNA microarrays, they then determined (by comparing each sample to the average of normal spinal cord samples) the gene expression changes that were unique to each type of MS lesion.

The results reveal that histopathologically different MS spinal cord lesions also exhibit distinct gene expression changes. Thus, gene lists, as set forth in the Tables below, result from the analysis of: (Table 1) MS spinal cord gray matter from a sample with minimal to no inflammation; (Table 2) MS spinal cord gray matter from a sample with lymphocytic inflammation, demyelination and axonal loss; (Table 3) MS spinal cord gray matter from a sample characterized by inflammation by lymphocytes and macrophages, and demyelination; (4) MS spinal cord gray matter from a sample with axonal loss; (Table 5) MS spinal cord white matter from a sample with minimal to no inflammation; (Table 6) MS spinal cord white matter from a sample with lymphocytic inflammation, demyelination and axonal loss; (Table 7) MS spinal cord white matter from a sample with inflammation by macrophages and demyelination; (Table 8) MS spinal cord white matter from a sample with inflammation by macrophages and lymphocytes and demyelination; and (Table 9) MS spinal cord white matter from a sample with axonal loss. In addition, the inventors provide tables of genes altered in (Table 10) a comparison of the group containing all MS spinal cord gray matter specimens against the group containing all normal gray matter specimens, and (Table 11) a comparison of the group containing all MS spinal cord white matter specimens against the group containing all normal white matter specimens. Also, genes altered commonly across all tables for gray matter (Table 12), white matter (Table 13), and gray & white matter (Table 14) are provided. Table 15 lists genes commonly altered across all comparisons of MS spinal cord white matter characterized by inflammation and demyelination, against normal spinal cord white matter tissues. Table 16, 17 and 18 list genes previously reported to be altered in MS brain tissues by, respectively, Lock et al. (2002) (indicated by one asterisk next to a gene identifier in Tables 1-15), Chabas et al. (2001) (indicated by two asterisks next to a gene identifier in Tables 1-15), and Whitney et al. (1999) (indicated by three asterisks next to a gene identifier in Tables 1-15), and also found by the inventors to be altered (i.e., coregulated and commonly shared) in MS spinal cord tissues. These genes are claimed as targets of treatment only in combination with other genes provided in the inventors' lists (and not in combination with other genes from lists by Lock et al. (2002), Chabas et al. (2001), or Whitney et al. (1999)).

I. Multiple Sclerosis

Multiple Sclerosis (MS) is one of the most common diseases of the central nervous system (brain and spinal cord). It is an inflammatory condition associated with demyelination, or loss of the myelin sheath. Myelin, a fatty material that insulates nerves, acts as insulator in allowing nerves to transmit impulses from one point to another. In MS, the loss of myelin is accompanied by a disruption in the ability of the nerves to conduct electrical impulses to and from the brain and this produces the various symptoms of MS, such as impairments in vision, muscle coordination, strength, sensation, speech and swallowing, bladder control, sexuality and cognitive function. The plaques or lesions where myelin is lost appear as hardened, scar-like areas. These scars appear at different times and in different areas of the brain and spinal cord, hence the term “multiple” sclerosis, literally meaning many scars.

Currently, there is no single laboratory test, symptom, or physical finding that provides a conclusive diagnosis of MS. To complicate matters, symptoms of MS can easily be confused with a wide variety of other diseases such as acute disseminated encephalomyelitis, Lyme disease, HIV-associated myelopathy, HTLV-I-associated myelopathy, neurosyphilis, progressive multifocal leukoencephalopathy, systemic lupus erythematosus, polyarteritis nodosa, Sjögren's syndrome, Behçet's disease, sarcoidosis, paraneoplastic syndromes, subacute combined degeneration of cord, subacute myelo-optic neuropathy, adrenomyeloneuropathy, spinocerebellar syndromes, hereditary spastic paraparesis/primary lateral sclerosis, strokes, tumors, arteriovenous malformations, arachnoid cysts, Arnold-Chiari malformations, and cervical spondylosis. Consequently, the diagnosis of MS must be made by a process that demonstrates findings that are consistent with MS, and also rules out other causes.

Generally, the diagnosis of MS relies on two criteria. First, there must have been two attacks at least one month apart. An attack, also known as an exacerbation, flare, or relapse, is a sudden appearance of or worsening of an MS symptom or symptoms which lasts at least 24 hours. Second, there must be more than one area of damage to central nervous system myelin sheath. Damage to sheath must have occurred at more than one point in time and not have been caused by any other disease that can cause demyelination or similar neurologic symptoms. MRI (magnetic resonance imaging) currently is the preferred method of imaging the brain to detect the presence of plaques or scarring caused by MS.

The diagnosis of MS cannot be made, however, solely on the basis of MRI. Other diseases can cause comparable lesions in the brain that resemble those caused by MS. Furthermore, the appearance of brain lesions by MRI can be quite heterogeneous in different patients, even resembling brain or spinal cord tumors in some. In addition, a normal MRI scan does not rule out a diagnosis of MS, as a small number of patients with confirmed MS do not show any lesions in the brain on MRI. These individuals often have spinal cord lesions or lesions which cannot be detected by MRI. As a result, it is critical that a thorough clinical exam also include a patient history and functional testing. This should cover mental, emotional, and language functions, movement and coordination, vision, balance, and the functions of the five senses. Sex, birthplace, family history, and age of the person when symptoms first began are also important considerations. Other tests, including evoked potentials (electrical diagnostic studies that may reveal delays in central nervous system conduction times), cerebrospinal fluid (seeking the presence of clonally-expanded immunoglobulin genes, referred to as oligoclonal bands), and blood (to rule out other causes), may be required in certain cases.

II. MS-Related Genes

In the following pages, the applicants set forth gene targets that may be targeted with therapies for MS. Also included are the particular probes utilized to identify these targets.

In the following tables, a positive value or a plus (+) sign for a log₁₀(ratio)-fold change value, or next to a probe set or gene name, indicates higher expression observed in patients with MS, as compared to healthy individuals. A negative value or a minus (−) sign for a log₁₀(ratio)-fold change value, or next to a probe set or gene name indicates lower expression observed in patients with MS, as compared to healthy individuals.

The inventors provide herein gene lists of altered mRNA transcripts in individual comparisons of gray or white matter tissue samples derived from MS spinal cord against normal spinal cord tissues (Tables 1-9). As stated, the inventors also provide tables of genes altered in a comparison of the entire group containing all MS spinal cord gray matter specimens against the entire group containing normal gray matter specimens (Table 10), and a comparison of the entire group containing all MS spinal cord white matter specimens against the entire group containing normal white matter specimens (Table 11). Finally, the inventors have identified genes that were dysregulated in each list from Tables 1-9, that had a significance of p<0.05. Then, a set of common genes that appeared in all gray/white lists was searched and means of log₁₀-fold changes, and p values calculated. Only 5 commonly dysregulated genes were identified in the MS gray matter comparison lists (shown as Table 12), while 23 dysregulated genes were commonly found in the MS white matter lists (shown as Table 13). Then, using the same method, another set of common genes across all lists (both gray and white) was also identified (shown as Table 14). Finally, since MS disease activity is characterized by inflammation and demyelination, a list of common genes shared by three individual comparisons of MS spinal cord white matter samples (all characterized by inflammation and demyelination) against normal spinal cord white matter samples was generated (Table 15). Finally, Tables 16, 17 and 18 show MS spinal cord tissue genes from the present invention that are commonly regulated and shared with the list of genes altered in MS tissues by Lock et al. (2002), Chabas et al. (2001), and Whitney et al. (1999). These genes are also shown by asterisk(s) preceding the probe set number in Tables 1-15, as follows: shared with the report by Lock et al. (one asterisk, Table 16), shared with the report by Chabas et al. (two asterisks, Table 17), and shared with the report by Whitney et al. (three asterisks, Table 18). The numbers under “Probe Sets” represent the GenBank accession numbers or, in some instances, an identifier provided by Affymetrix.

TABLE 1
Gene targets in MS spinal cord gray matter from a sample with minimal to no
histological inflammation.

| Probe Set | Gene description | log10 (ratio) fold change |
|---|---|---|
| M77829 | Channel-like integral membrane prot (CHIP28); Also: S73482 | 2.1973319000 |
| X95406 | Cyclin E | −2.4820424000 |
| U09937 | Urokinase-type plasminogen activator receptor; Also: X51675 | −2.4221999000 |
| M25280 | Lymph node homing receptor | 2.0461048000 |
| *X64072 | CD18; Also: M15395 | 2.0293838000 |
| M21305 | Alpha satellite and satellite 3 junction DNA sequence | −2.2956496000 |
| M68864 | ORF | −2.1876265000 |
| M64788 | GTPase activating protein (rap1GAP) | 1.9611837000 |
| *M87789 | Hybridoma H210 anti-hepatitis A IgG V, C, CDR regions | 1.9385197000 |
| U78793 | Folate receptor alpha (hFR)/U78793 | −2.1603560000 |
| D55696 | Cysteine protease | 1.9194646000 |
| U02031 | Sterol regulatory element binding protein-2 | 1.9049812000 |
| M98045 | Folylpolyglutamate synthetase | −2.1298107000 |
| X57351 | 1-8D from interferon-inducible family | −2.1186367000 |
| D86971 | KIAA0217 | 1.8382249000 |
| M37457 | Na+, K+-ATPase catalytic subunit alpha-III isoform | 1.8061800000 |
| M37755 | Pregnancy-specific beta-1-glycoprotein PSGGA | −2.0861373000 |
| X53414 | Peroxisomal L-alanine:glyoxylate aminotransferase | −2.0852014000 |
| J04501 | Muscle glycogen synthase | 1.8014037000 |
| D45906 | LIMK-2 | −2.0813473000 |
| U60800 | Semaphorin (CD100) | 1.7899331000 |
| Z84718 | DNA on chromosome 22q 11.2-qter contains GSTT1-2 | 1.7710628000 |
| M13207 | Granulocyte-macrophage colony-stimulating factor (CSF1) | −2.0635210000 |
| M97252 | Kallmann syndrome (KAL) | 1.7542944000 |
| U39573 | Salivary peroxidase | −2.0552349000 |
| J03600 | Lipoxygenase | −2.0488301000 |
| U56816 | Kinase Myt1 (Myt1) | −2.0437060000 |
| X17360 | HOX 5.1 protein | 1.7295697000 |
| Z19002 | PLZF kruppel-like zinc finger protein | −2.0348289000 |
| U70732 | Glutamate pyruvate transaminase (GPT) | 1.7155044000 |
| L02648 | (clone V6) transcobalamin II (TCN2) | −2.0310043000 |
| M16707 | Histone H4; clone FO108 | −2.0251522000 |
| L14812 | Retinoblastoma related protein (p107) | −2.0221189000 |
| U73799 | Dynactin/U73799 | 1.6954817000 |
| D28383 | ATP synthase B chain | 1.6825045000 |
| D50550 | LLGL | 1.6771760000 |
| M95929 | Homeobox protein (PHOX1) | 1.6766936000 |
| HG1019-HT1019 | Serine Kinase Psk-H1 | −2.0052342000 |
| U91327 | Chromosome 12p15 BAC clone CIT987SK-99D8 sequence | 1.6707096000 |
| M64231 | Spermidine synthase | −1.9982048000 |
| U79725 | A33 antigen precursor | −2.0023281000 |
| D16583 | L-histidine decarboxylase | 1.6622855000 |
| U68723 | Checkpoint suppressor 1 | 1.6419879000 |
| U94747 | WD repeat protein HAN11/U94747 | 1.6329632000 |
| U49089 | Neuroendocrine-dlg (NE-dlg) | −1.9643186000 |
| U22970 | 16-Jun (interferon-inducible peptide precursor) | −1.9576671000 |
| X86681 | Nucleolar protein HNP36 | −1.9585042000 |
| HG2147-HT2217 | Mucin 3, Intestinal/M55405 | −1.9540012000 |
| X14474 | Microtubule-associated tau protein | 1.5998831000 |
| D55638 | B-cell pseudoautosomal boundary-like sequence | −1.9438653000 |
| M58597 | ELAM-1 ligand fucosyltransferase (ELFT) | −1.9472499000 |
| U37519 | Aldehyde dehydrogenase (ALDH8) | −1.9341827000 |
| HG651-HT5209 | Adducin, Alpha Subunit; Also: Z68280_2, HG651-HT4201 | 1.5820634000 |
| M13928 | X64467_rna1 and others | 1.5757650000 |
| M63573 | Secreted cyclophilin-like protein (SCYLP) | 1.5763414000 |
| U48861 | Beta 4 nicotinic acetylcholine receptor subunit | 1.5711263000 |
| M12125 | Fibroblast muscle-type tropomyosin | −1.9227255000 |
| Z80780 | H2B/h/Z80780 | 1.5641730000 |
| D84454 | UDP-galactose translocator | 1.5618030000 |
| X96783 | Syt V | 1.5599066000 |
| X13810 | OTF-2 lymphoid-specific transcription factor; Also: M36542 | −1.9130850000 |
| X52611 | Transcription factor AP-2; Also: HG2465-HT4871, M36711 | −1.9158613000 |
| AF000545 | Putative purinergic receptor P2Y10 | −1.9043097000 |
| L35240 | Enigma | 1.5440680000 |
| X81420 | hHKb1 protein | 1.5432976000 |
| D28532 | Renal Na+-dependent phosphate cotransporter | −1.8974209000 |
| X63692 | DNA (cytosin-5)-methyltransferase | 1.5308398000 |
| J05448 | RNA polymerase subunit hRPB 33 | 1.5199493000 |
| U31628 | Interleukin-15 receptor alpha chain precursor (IL15RA) | 1.5214458000 |
| D43968 | AML1b protein | −1.8803133000 |
| M13232 | Factor VII serine protease precursor; Also: J02933 | −1.8815986000 |
| U57450 | EPC-1 | −1.8814560000 |
| U29091 | Selenium-binding protein (hSBP)/U29091 | 1.5158738000 |
| Y07755 | S100A2 | −1.8752784000 |
| X16667 | HOX2G from the Hox2 locus | −1.8693784000 |
| HG3033-HT3194 | Spliceosomal protein Sap 62 | 1.5048795000 |
| X52213 | ltk; Also: D16105 | 1.5037907000 |
| X01038 | Fetal apolipoprot AI precursor; Also: X07496 | −1.8629658000 |
| U64197 | Chemokine exodus | −1.8589881000 |
| HG2709-HT2805 | Serine/Threonine Kinase | 1.4913617000 |
| D29642 | KIAA0053 | 1.4694538000 |
| L11708 | 17 beta hydroxysteroid dehydrogenase type 2 | −1.8364032000 |
| D38502 | PMS4 (yeast PMS1 homolog) | 1.4631461000 |
| *M85220 | Heavy chain disease IgA chain CH3 region | 1.4556061000 |
| M60299 | Alpha-1 collagen type II s 1 2 and 3 | −1.8246952000 |
| M73481 | Gastrin releasing peptide receptor (GRPR) | −1.8188030000 |
| D26350 | Type 2 inositol 1 4 5-trisphosphate receptor | 1.4471580000 |
| X90840 | Axonal transporter of synaptic vesicles | −1.8153287000 |
| D59253 | NCBP interacting protein 1 | 1.4369573000 |
| X17651 | Myf-4 myogenic determination factor | 1.4310639000 |
| M95178 | Non-muscle alpha-actinin | 1.4240645000 |
| L32866 | Effector cell protease receptor-1 (EPR-1) | −1.8044802000 |
| X87871 | Hepatocyte nuclear factor 4b; Also: X87870, Z49825 | −1.8050759000 |
| HG2510-HT2606 | Ras-Specific Guanine Nucleotide-Releasing Factor | 1.4210614000 |
| M60298 | Erythrocyte membrane protein band 42 (EPB42) | 1.4132998000 |
| AF003743 | Delayed rectifier potassium channel (KVLQT1-Iso5) | −1.7940211000 |
| U07139 | Voltage-gated calcium channel beta subunit | 1.4040622000 |
| U33429 | K+ channel beta 2 subunit | 1.4052248000 |
| L00205 | K6b epidermal keratin type II | −1.7875490000 |
| U04847 | Ini1 | −1.7896688000 |
| U58090 | Hs-cul-4A | 1.3944517000 |
| U07919 | Aldehyde dehydrogenase 6 | −1.7871717000 |
| X59798 | PRAD1 cyclin | 1.3906515000 |
| U78190 | GTP cyclohydrolase I feedback regulatory protein | −1.7774268000 |
| D79998 | KIAA0176 | −1.7700231000 |
| D82344 | NBPhox | −1.7704838000 |
| HG919-HT919 | Dna Polymerase Epsilon Catalytic Subunit | −1.7714956000 |
| U03911 | Mutator (hMSH2) | 1.3594190000 |
| U24169 | JTV-1 (JTV-1) | 1.3614459000 |
| U71364 | Serine protase inhibitor (P19) | 1.3598355000 |
| S79781 | WT1/S79781 | −1.7641855000 |
| M31606 | Phosphorylase kinase (PSK-C3) | 1.3473300000 |
| M95740 | Alpha-L-iduronidase | 1.3443923000 |
| M27492 | Interleukin 1 receptor | −1.7547305000 |
| AD000092 | RAD23A homolog | 1.3262746000 |
| Y08613 | Nup88/Y08613 | −1.7490596000 |
| M59820 | Granulocyte colony-stimulating factor receptor (CSF3R) | 1.3031961000 |
| L04751 | Cytochrome p-450 4A (CYP4A) | −1.7450748000 |
| U28281 | Secretin receptor | −1.7464396000 |
| X63380 | RSRFR2 | 1.3004888000 |
| U13737 | Cysteine protease CPP32 isom alpha | −1.7422340000 |
| M80647 | Thromboxane synthase | 1.2811352000 |
| X54871 | Ras-related protein Rab5b | 1.2810334000 |
| X14445 | Int-2 protooncogene | −1.7359979000 |
| HG1996-HT2044 | Guanine Nucleotide-Binding protein Rap2 | 1.2741578000 |
| M95809 | Basic TRANSCRIPTION FACTOR 62 kD subunit (BTF2) | 1.2684286000 |
| D43772 | GRB-7 SH2 domain | −1.7320317000 |
| M12886 | T-cell receptor active beta-chain | −1.7301764000 |
| M18700 | D00306, M16630, M18692 | −1.7273379000 |
| U49974 | Mariner2 transposable element, complete consensus/U49974 | −1.7266253000 |
| HG3748-HT4018 | Basic Transcription Factor 44 Kda Subunit | 1.2504200000 |
| AF015950 | Telomerase reverse transcriptase | 1.2348010000 |
| HG172-HT3924 | Spermidine/Spermine N1-Acetyltransferase | −1.7147488000 |
| HG4749-HT5197 | Carnitine Calcium-Binding protein Mitochondrial | −1.7155856000 |
| M55905 | Mitochondrial NAD(P)+ dependent malic enzyme | 1.2188888000 |
| U82987 | Bcl-2 binding component 3 (bbc3) | 1.2105620000 |
| M21984 | (clone PWHTnT16) skeletal muscle Troponin T | −1.7104347000 |
| U27333 | Alpha (1,3) fucosyltransferase (FUT6), major transcript I | −1.7091639000 |
| X52228 | Secreted epithelial tumour mucin antigen | −1.7084421000 |
| U11313 | Sterol carrier protein-X/sterol carrier protein-2 (SCP-X/SCP-2) | 1.2041200000 |
| Y08766 | Splicing factor, SF1-Bo isoform; Also: L49380 | 1.2043797000 |
| Z38133 | Myosin; Also: M36769 | 1.2066376000 |
| X06389 | Synaptophysin (p38) | 1.1958997000 |
| ***J05037 | Serine dehydratase | 1.1836070000 |
| U66619 | SWI/SNF complex 60 KDa subunit (BAF60c) | 1.1869136000 |
| AB000462 | SH3 binding RES4-23A | −1.6994041000 |
| M34344 | Platelet glycoprotein IIb (GPIIb) | −1.6981938000 |
| U03399 | T-complex protein 10A (TCP10A) | −1.6989700000 |
| X52599 | Beta nerve growth factor | −1.6989700000 |
| M14159 | T-cell receptor beta-chain J2.1 | −1.6935071000 |
| HG880-HT880 | MUC6 | −1.6893089000 |
| L27080 | Melanocortin 5 receptor (MC5R) | −1.6898081000 |
| X53795 | R2 inducible membrane protein | −1.6877853000 |
| J05459 | Glutathione transferase M3 (GSTM3) | 1.1278244000 |
| M14091 | Thyroxine-binding globulin | −1.6859655000 |
| U01157 | Glucagon-like peptide-1 receptor with CA dinucleotide repeat | 1.1080076000 |
| M74542 | Aldehyde dehydrogenase type III (ALDHIII) | −1.6798819000 |
| D28423 | Pre-splicing factor SRp20 | 1.1074088000 |
| X97748 | PTX3/X97748; Also: M31166 | 1.0958364000 |
| M55621 | N-acetylglucosaminyltransferase I (GlcNAc-TI) | −1.6770363000 |
| Y07829 | RING protein | −1.6729056000 |
| Y09561 | P2X7 receptor | 1.0883800000 |
| S83325 | Aspartyl(asparaginyl)beta-hydroxylase; Also: U03109 | 1.0849797000 |
| D50920 | KIAA0130 | −1.6678030000 |
| Y10262 | EYA3/Y10262; Also: U81602 | −1.6676864000 |
| U80457 | TRANSCRIPTION FACTOR SIM2 short form | 1.0646428000 |
| L41351 | Prostasin | −1.6632296000 |
| J03068 | DNF1552 (lung) | 1.0594372000 |
| U62433 | Nicotinic acetylcholine receptor alpha4 subunit precursor | 1.0592429000 |
| L76528 | Presenilin 1 (PS1; S182); Also: L76517 | 1.0524293000 |
| L38933 | Putative with an open reading frame | −1.6565773000 |
| S82472 | Polymerase beta/S82472 | −1.6539357000 |
| M14949 | R-ras | 1.0389477000 |
| U46746 | Dystrobrevin-epsilon; Also: U46744 | 1.0359475000 |
| X78992 | ERF-2 | 1.0341524000 |
| X76105 | DAP-1 | 1.0298785000 |
| L03840 | Fibroblast growth factor receptor 4 (FGFR4); Also: X57205 | −1.6517624000 |
| M25077 | SS-A/Ro ribonucleoprot autoantigen 60 kd subunit | 1.0221579000 |
| X99479 | NK receptor, clone 12.11C-Also Similar To: X93596, L76672 | −1.6450537000 |
| D13168 | Endothelin-B receptor | 1.0139885000 |
| HG511-HT511 | Ras Inhibitor Inf | 1.0134230000 |
| L13698 | Growth-arrest-specific protein (gas) | 1.0174481000 |
| *U21090 | DNA polymerase delta small subunit | 1.0132654000 |
| U36448 | Ca2+-dependent activator protein secretion | 1.0153598000 |
| U82979 | Ig-like transcript-3 | −1.6332159000 |
| L07592 | Peroxisome proliferator activated receptor | −1.6308091000 |
| U15932 | Dual-specificity protein phosphatase | −1.6301735000 |
| U32674 | Orphan receptor GPR9 (GPR9); Also: X95876 | −1.6318241000 |
| X68090 | Fc-gamma-RIIA IgG Fc receptor class IIA/X68090 | −1.6294096000 |
| D31764 | KIAA0064 | 0.9738912400 |
| M27543 | Guanine nucleotide-binding protein (Gi) alpha subunit | 0.9751461800 |
| Z11695 | 40 kDa protein kinase related to rat ERK2 | 0.9731278500 |
| D31886 | KIAA0066 | 0.9686520200 |
| HG3985-HT4255 | Cpg-Enriched Dna Clone E04 | −1.6245399000 |
| D10495 | Protein kinase C delta-type | 0.9660821500 |
| AB000896 | Cadherin FIB2 | −1.6193542000 |
| U32581 | Lambda/iota-prot kinase C-interacting protein | −1.6184404000 |
| U63090 | Gal beta-13 GalNAc alpha-23 sialyltransferase (ST3Gal II) | −1.6209873000 |
| *D10704 | Choline kinase | 0.9532953000 |
| D90359 | CCG1 | 0.9501715500 |
| D29675 | iNOS | −1.6131809000 |
| M63904 | G-alpha 16 protein | −1.6134650000 |
| D80009 | KIAA0187 | 0.9330532100 |
| Y10260 | EYA1 | −1.6079909000 |
| Z80345 | SCAD; Also: M26393 | −1.6091941000 |
| U49188 | Placenta (Diff33) | 0.9315474000 |
| U23430 | Cholecystokinin type A receptor (CCK-A); Also: L19315 | 0.9255532700 |
| M15465 | Pyruvate kinase type L; Also: D13243 | −1.6032797000 |
| U17077 | BENE | −1.6035503000 |
| U41344 | Prolargin (PRELP) | −1.6028735000 |
| Y08836 | HRX-like protein/Y08836 | −1.6028735000 |
| L21993 | Adenylyl cyclase | 0.9201585600 |
| L37127 | (clone mf18) RNA polymerase II | 0.9208968900 |
| U89916 | Putative OSP like protein | 0.9140545300 |
| U55054 | K-Cl cotransporter (hKCC1) | 0.9122979900 |
| L38969 | Thrombospondin 3 (THBS3) | −1.6019243000 |
| HG3517-HT3711 | Alpha-1-Antitrypsin | −1.5969543000 |
| U08021 | Nicotinamide N-methyltransferase (NNMT) | −1.5934245000 |
| U82613 | DNA-binding protein ABP/ZF | −1.5942544000 |
| X66141 | Cardiac ventricular myosin light chain-2 | −1.5938900000 |
| X78687 | G9 encoding sialidase | −1.5934139000 |
| M81182 | Peroxisomal 70 kD membrane protein; Also: X83467_rna1 | 0.8950389800 |
| U09646 | Carnitine palmitoyltransferase II precursor (CPT1) | 0.8888069700 |
| *U64573 | Connexin43 gap junction protein (connexin43)/U64573 | 0.8848286700 |
| U18237 | ATP-binding cassette protein 06B09 clone | −1.5917600000 |
| X99720 | TPRC | 0.8798514200 |
| Z46632 | HSPDE4C1 3,5-cyclic AMP phosphodiesterase | 0.8815503100 |
| S81294 | DCC = deleted in colorectal cancer/S81294 | −1.5848963000 |
| X77307 | 5-HT2B serotonin receptor | −1.5841898000 |
| X66362 | PCTAIRE-3 serine/threonine protein kinase | 0.8690051700 |
| D42053 | KIAA0091 | 0.8599566400 |
| U35139 | NECDIN related protein | 0.8591271000 |
| U93049 | SLP-76 associated protein | 0.8613913500 |
| D43947 | KIAA0100 | 0.8530723400 |
| M13903 | Involucrin | 0.8571154000 |
| M29277 | Isolate JuSo MUC18 glycoprot (3 variant); Also: M28882 | 0.8570771000 |
| D79989 | KIAA0167 | −1.5764853000 |
| U78876 | MEK kinase 3 | −1.5733068000 |
| Z83805 | Axonemal dynein heavy chain (ID hdhc8)/Z83805 | −1.5731618000 |
| U22028 | Cytochrome P450 (CYP2A13) | 0.8496834500 |
| D87433 | KIAA0246 | −1.5683484000 |
| U40002 | Hormone-sensitive lipase testicular isoform; Also: L11706 | −1.5721452000 |

TABLE 2
Gene targets in MS spinal cord gray matter from a sample with lymphocytic
inflammation, demyelination and axonal loss.

| Probe set | Gene description | log10 (ratio) fold change |
|---|---|---|
| *M87789 | Anti-hepatitis A IgG V, C, CDR regions; Also: J00221_2 | 3.1221503000 |
| Y00067 | Neurofilament subunit M (NF-M) | −3.0995382000 |
| D13643 | KIAA0018 | −3.0496210000 |
| *M63438 | Ig rearranged gamma chain, V-J-C region; Also: X96754 | 2.6660416000 |
| L10678 | Profilin II | −2.6086865000 |
| *U62317 | Hypothetical protein 384D8_7 | 2.2480959000 |
| M22538 | Mitochondrial NADH-ubiquinone reductase 24 Kd subunit | −2.5507938000 |
| X99657 | Protein containing SH3 domain SH3GL2 | 2.2324879000 |
| X98225 | Gastrin-binding protein/X98225 | 2.2077690000 |
| L07597 | Ribosomal protein S6 kinase 2 (RPS6KA2) | 2.2060088000 |
| D13705 | Fatty acids omega-hydroxylase (cytochrome P-450HKV) | −2.4579765000 |
| L14565 | Peripherin (PRPH)s 1-9 | −2.4614422000 |
| U57341 | Neurofilament triplet L protein/U57341 | −2.4386017000 |
| U60644 | HU-K4 | −2.4171186000 |
| Y00757 | Polypeptide 7B2 | −2.3941670000 |
| D50550 | LLGL | 2.1022931000 |
| U92457 | Metabotropic glutamate receptor 4; Also: X80818 | 2.1004650000 |
| D63484 | KIAA0150 | 2.0939467000 |
| HG3033-HT3194 | Spliceosomal protein Sap 62 | 2.0822352000 |
| M27749 | Ig-related 14.1 protein | 2.0722499000 |
| M22632 | Mitochondrial aspartate aminotransferase | −2.3186893000 |
| X87870 | Hepatocyte nuclear factor 4a | 2.0546131000 |
| U18244 | Excitatory amino acid transporter 4 | 2.0463000000 |
| *X00734 | Beta-tubulin (5-beta) with ten Alu family members | −2.2901738000 |
| M22976 | Cytochrome b5 | −2.2794103000 |
| M57609 | DNA-binding protein (GLI3) | −2.2654959000 |
| D50663 | TCTEL1 | −2.2489536000 |
| J05073 | Phosphoglycerate mutase (PGAM-M) | 1.9672499000 |
| U82987 | Bcl-2 binding component 3 (bbc3) | 1.9497120000 |
| L35240 | Enigma | 1.9412629000 |
| S83390 | T3 receptor-associating cofactor-1; Also: U37146 | 1.9278533000 |
| L07738 | DHP-sensitive calcium channel gamma subunit (CACNLG) | 1.9250541000 |
| D43767 | Chemokine | −2.2221635000 |
| L40397 | (clone S31i125) | −2.2061848000 |
| M36542 | Lymphoid-specific transcription factor; Also: X13810, X13809 | −2.2043574000 |
| U11877 | Interleukin-8 receptor type B (IL8RB)/U11877 | 1.8893017000 |
| J00123 | Enkephalin | −2.1938895000 |
| X92896 | ITBA2 protein | −2.1926490000 |
| X15331 | Phosphoribosylpyrophosphate synthetase subunit one | −2.1855067000 |
| Z21488 | Contactin | −2.1837679000 |
| U49973 | Tigger 1 transposable element | 1.8618330000 |
| D16583 | L-histidine decarboxylase | 1.8518696000 |
| U52969 | PEP19 (PCP4) | −2.1730770000 |
| **X05299 | (˜95%) major centromere autoantigen CENP-B | 1.8387444000 |
| X01630 | Argininosuccinate synthetase | −2.1602058000 |
| D50402 | NRAMP1 | 1.8214780000 |
| J00220 | IGHA1 from Ig germline H-chain G-E-A region A: gamma-3 5 | 1.8210038000 |
| U16031 | TRANSCRIPTION FACTOR IL-4 Stat | 1.8178958000 |
| X67325 | p27 | −2.1546522000 |
| M29927 | Ornithine aminotransferase | −2.1375915000 |
| M87860 | S-lac lectin L-14-II (LGALS2) | 1.7914660000 |
| M58509 | FDXR (adrenodoxin reductase); Also: HG2836-HT2962 | 1.7833611000 |
| L47345 | Elongin A | −2.1360464000 |
| M13755 | Interferon-induced 17-kDa/15-kDa protein | −2.1340391000 |
| HG3928-HT4198 | SFTPA2D | 1.7625109000 |
| HG2348-HT2444 | Peptide Yy; Also: D13897 | 1.7609311000 |
| L34587 | RNA polymerase II elongation factor SIII p15 subunit | −2.1126469000 |
| X05608 | Neurofilament subunit NF-L | −2.1160768000 |
| M95178 | Non-muscle alpha-actinin | 1.7450748000 |
| D38128 | IP prostacyclin receptor | 1.7411516000 |
| HG2604-HT2700 | Pan-2 | 1.7415455000 |
| U42408 | Ladinin (LAD) | 1.7395723000 |
| Z33905 | 43 kD acetylcholine receptor-associated protein (Rapsyn) | 1.7335985000 |
| L41143 | Expressed pseudo TCTA at t(1;3) translocation site | −2.1054392000 |
| HG4128-HT4398 | Anion Exchanger 3 Cardiac Isom | 1.7299743000 |
| X64037 | RNA polymerase II associated protein RAP74 | −2.0997238000 |
| X52638 | 6-phosphofructo-2-kinase/fructose-26-bisphosphatase | −2.0953001000 |
| U72671 | Telencephalin precursor | 1.7172543000 |
| D85758 | DROER homolog | −2.0905666000 |
| *L26339 | Autoantigen | 1.7041505000 |
| U50360 | Calcium, calmodulin-dependent protein kinase II gamma | −2.0863598000 |
| X53414 | Peroxisomal L-alanine: glyoxylate aminotransferase | −2.0852014000 |
| U89336 | Notch 4 | 1.6941903000 |
| U79255 | X11 protein | −2.0789097000 |
| X99142 | Hair keratin hHb6 | −2.0812573000 |
| D14663 | KIAA0107 | −2.0728011000 |
| X99897 | P/Q-type calcium channel alpha1 subunit; Also: U79663 | 1.6798819000 |
| J00124 | 50 kDa type I epidermal keratin | −2.0680931000 |
| X04706 | Homeobox (clone HHO.c13); Also: X17360 | 1.6702459000 |
| M13207 | Granulocyte-macrophage colony-stimulating factor (CSF1) | −2.0635210000 |
| X02761 | Fibronectin (FN precursor); Also: HG3044-HT2527 | −2.0637086000 |
| U61734 | Protein trafficking protein (S31iii125); Also: L40397 | −2.0620176000 |
| D50532 | Macrophage lectin 2 | 1.6536841000 |
| L09708 | Complement component 2 (C2) allele b | 1.6573649000 |
| M74509 | HG4045-HT4315 and others | −2.0537025000 |
| S72487 | Orf1 to PD-ECGF/TPorf2 to PD-ECGF/TP | −2.0528382000 |
| X17094 | Furin | 1.6503075000 |
| *D13988 | Rab GDI | −2.0496540000 |
| X59842 | PBX2; Also: U89336_2, D28769_1, X80700 | −2.0479877000 |
| X02958 | Interferon alpha IFN-alpha 6 | 1.6349808000 |
| U35340 | Beta B1-crystallin | 1.6263404000 |
| L02648 | (clone V6) transcobalamin II (TCN2) | −2.0310043000 |
| U23803 | Heterogeneous ribonucleoprotein A0 | −2.0311711000 |
| U91316 | Acyl-CoA thioester hydrolase | −2.0287237000 |
| M16707 | Histone H4; clone FO108 | −2.0251522000 |
| M86826 | IGF binding protein complex acid-labile subunit a | 1.6117233000 |
| X81637 | Clathrin light chain b | 1.6111921000 |
| U25801 | Tax1 binding protein | 1.6031444000 |
| M96738 | Somatostatin receptor subtype 3 (SSTR3); Also: Z86000 | 1.5993371000 |
| D49817 | Fructose 6-phosphate 2-kinase/fructose 2 6-bisphosphatase | −2.0132587000 |
| U73499 | Hepatic nuclear factor 1-alpha (TCF-1-alpha)/U73499 | 1.5929617000 |
| HG2709-HT2805 | Serine/Threonine Kinase | 1.5910646000 |
| HG4115-HT4385 | Olfactory Receptor Or17-210 | 1.5899496000 |
| S77893 | GPSAT = glycophorin SAT; Also: L31860 | 1.5831988000 |
| D50913 | KIAA0123 | −2.0061450000 |
| M33882 | p78 protein | −2.0057702000 |
| X77748 | Metabotropic glutamate receptor type 3 | −2.0060915000 |
| HG3991-HT4261 | Cpg-Enriched Dna, Clone E18 | 1.5809250000 |
| *U62317 | Hypothetical protein 384D8_7 | 1.5818566000 |
| *J03263 | Lysosome-associated membrane glycoprotein (lamp A) | −2.0018959000 |
| M81780 | SMPD1 | 1.5735256000 |
| X70070 | Neurotensin receptor | 1.5770137000 |
| M64925 | Palmitoylated erythrocyte membrane protein (MPP1) | −1.9929399000 |
| X07315 | PP15 (placental protein 15) | −1.9968399000 |
| U82279 | Ig-like transcript 2 | 1.5717088000 |
| K03021 | Tissue plasminogen activator (PLAT) | −1.9892829000 |
| X06956 | HALPHA44 alpha-tubulin | −1.9917528000 |
| S69370 | PAX3B = transcription factor; Also: S69369 | 1.5611014000 |
| S52969 | Alpha-1.3 fucosyltransferase/FucT-III and Fuct-VI | −1.9849209000 |
| U32645 | Myeloid elf-1 like factor (MEF) | 1.5563025000 |
| HG2259-HT2348 | Tubulin, Alpha 1; Also: X06956 | −1.9746844000 |
| M77810 | TRANSCRIPTION FACTOR GATA-2 | 1.5440680000 |
| U01828 | Microtubule-associated protein 2 (MAP2) | 1.5378191000 |
| *X64072 | CD18; Also: M15395 | 1.5346606000 |
| X77567 | InsP3 5-phosphatase; Also: Z31695 | 1.5360159000 |
| M21574 | Platelet-derived growth factor receptor alpha (PDGFRA) | −1.9621325000 |
| U22970 | 16-Jun (interferon-inducible peptide precursor); Also: U22970 | −1.9576671000 |
| U60521 | Protease proMch6 (Mch6) | −1.9583249000 |
| D38163 | a1(XIX) collagen chain; Also: U09279 | 1.5232039000 |
| D86549 | p97 homologous protein | −1.9537597000 |
| L36983 | Dynamin (DNM) | −1.9527319000 |
| S67247 | Smooth muscle myosin heavy chain isoform Smemb | 1.5204835000 |
| U73799 | Dynactin/U73799 | 1.5198280000 |
| X98258 | M-phase phosphoprotein mpp9 | 1.5211381000 |
| U18671 | Stat2 | −1.9471273000 |
| U24183 | Phosphofructokinase (PFKM); Also: HG1849-HT1878 | −1.9432471000 |
| L41939 | (clone FBK III 11c) protein-tyrosine kinase (DRT) | 1.5047354000 |
| L08069 | Heat shock protein E coli DnaJ homolog | −1.9388323000 |
| HG4458-HT4727 | Ig Heavy Chain Vdjc Regions | 1.4976206000 |
| U46767 | Monocyte chemoattractant protein-4 precursor (MCP-4) | 1.5006024000 |
| U17886 | Succinate dehydrogenase iron-protein subunit (sdhB) | −1.9363881000 |
| S71018 | Cyclophilin C | 1.4892552000 |
| D14874 | Adrenomedullin | −1.9296743000 |
| M62843 | Antigen of paraneoplastic sensory neuronopathy patients | −1.9305033000 |
| U28488 | Putative G protein-coupled receptor (AZ3B); Also: U62027 | 1.4864470000 |
| X82324 | Brain 4 | 1.4828736000 |
| X52008 | Strychnine binding subunit of inhibitory glycine receptor | −1.9199275000 |
| *M35999 | Platelet glycoprotein IIIa (GPIIIa) | 1.4727564000 |
| U08316 | Insulin-stimulated protein kinase 1 (ISPK-1) | −1.9153998000 |
| X13810 | OTF-2 lymphoid-specific transcription factor; Also: M36542 | −1.9130850000 |
| M89470 | Paired-box protein (PAX2); Also: L25597 | −1.9122221000 |
| X03072 | Int-1 mammary oncogene | −1.9080827000 |
| X66363 | PCTAIRE-1 serine/threonine protein kinase | −1.9106244000 |
| L14848 | MHC class I-related protein; Also: X91625, U65416, X92841 | 1.4664804000 |
| X97748 | PTX3/X97748; Also: M31166 | 1.4629523000 |
| M21056 | Pancreatic phospholipase A-2 (PLA-2) | 1.4616772000 |
| M92934 | Connective tissue growth factor | −1.9045194000 |
| U07620 | MAP kinase | −1.9057284000 |
| U41668 | Deoxyguanosine kinase | −1.9062003000 |
| U95006 | D9 splice variant A | −1.9062677000 |
| M60891 | Uroporphyrinogen decarboxylase (URO-D)/M60891 | 1.4528033000 |
| M86406 | Skeletal muscle alpha 2 actinin | −1.8953535000 |
| *Y12711 | Putative progesterone binding protein | −1.8947313000 |
| X15675 | pTR7 repetitive sequence/X15675 | 1.4440448000 |
| Z46788 | Cylicin II | 1.4471580000 |
| M99438 | Transducin-like enhancer protein (TLE3) | 1.4392274000 |
| L78833 | Ifp35 from Rho7 vatl and BRCA1 | −1.8836614000 |
| M94077 | Loricrin | 1.4364518000 |
| U62433 | Nicotinic acetylcholine receptor alpha4 subunit precursor | 1.4370998000 |
| K03183 | Chorionic gonadotropin beta subunit | −1.8803276000 |
| M65085 | Follicle stimulating hormone receptor | 1.4251925000 |
| X80026 | B-cam | −1.8752784000 |
| M60459 | Erythropoietin receptor | 1.4224257000 |
| M64595 | Small G protein (Gx) | 1.4207806000 |
| U19247 | Interferon-gamma receptor alpha chain | −1.8687913000 |
| X78992 | ERF-2 | 1.4167905000 |
| L41147 | 5-HT6 serotonin receptor | −1.8638257000 |
| X77794 | Cyclin G1 | −1.8655481000 |
| U64197 | Chemokine exodus | −1.8589881000 |
| U81607 | Gravin | −1.8604129000 |
| *L25270 | XE169 | 1.4063065000 |
| U72507 | 40871 sequence | 1.4070380000 |
| L16991 | Thymidylate kinase (CDC8) | −1.8551404000 |
| L31573 | Sulfite oxidase | 1.3947839000 |
| L44140 | DNL1L from chromosome X region; Also: X90392 | 1.3935752000 |
| M63573 | Secreted cyclophilin-like protein (SCYLP) | 1.3953264000 |
| X51804 | PMI a putative receptor protein | −1.8500333000 |
| X87176 | 17-beta-hydroxysteroid dehydrogenase | −1.8495730000 |
| U56833 | VHL binding protein-1 (VBP-1) | −1.8445548000 |
| U53174 | Cell cycle checkpoint control protein | 1.3829171000 |
| D10925 | HM145 | 1.3812916000 |
| M11321 | Group-specific component vitamin D-binding protein | 1.3820170000 |
| X90999 | Glyoxalase II | −1.8350561000 |
| HG1139-HT4910 | Fk506-Binding protein | 1.3765770000 |
| HG4683-HT5108 | Tumor Necrosis Factor Receptor 2 Associated protein Trap3 | 1.3756148000 |
| M17252 | Cytochrome P450c21 | 1.3773315000 |
| X15954 | MBP1; Also: X15422 | 1.3747483000 |
| U76456 | Tissue inhibitor of metalloproteinase 4 | 1.3694342000 |
| L38487 | Estrogen receptor-related protein (hERRa1) | −1.8276358000 |
| AF000234 | P2x purinoceptor | 1.3626709000 |
| HG4051-HT4321 | Choline Acetyltransferase | 1.3626709000 |
| M27878 | DNA binding protein (HPF2) | 1.3645510000 |
| U65011 | Preferentially expressed antigen of melanoma (PRAME) | 1.3652493000 |
| X52896 | Dermal fibroblast elastin; Also: HG2994-HT4851 | 1.3636120000 |
| Z35278 | PEBP2aC1 acute myeloid leukaemia | 1.3626709000 |
| X89067 | Trpc2 transcript (possible pseudo) | 1.3586961000 |
| D78275 | Proteasome subunit p42 | −1.8189678000 |
| L24804 | (p23) | −1.8218409000 |
| M24594 | Interferon-inducible 56 Kd protein | −1.8170693000 |
| X90840 | Axonal transporter of synaptic vesicles | −1.8153287000 |
| HG4557-HT4962 | Small Nuclear Ribonucleoprotein U1, 1snrp | 1.3434086000 |
| S76067 | CNG2 = cyclic nucleotide-gated cation channel/S76067 | 1.3456620000 |
| D85433 | MURR1 | −1.8097280000 |
| X12458 | P3 | 1.3414345000 |
| D28383 | ATP synthase B chain | 1.3369167000 |
| Z84721 | DNA from 16p13.3 Contains alpha and zeta globin | −1.8031156000 |
| U80017 | Survival motor neuron protein | 1.3281136000 |
| D42087 | KIAA0118 | −1.8007171000 |
| J05582 | Pancreatic mucin; Also: J05581 | −1.8004593000 |
| L00634 | Farnesyl-protein transferase alpha-subunit; Also: L10413 | −1.7992544000 |
| S80335 | Integrin beta 7 subunit | 1.3252515000 |
| U67849 | Beta-galactoside alpha26-sialyltransferase (SIAT1)/U67849 | 1.3200240000 |
| HG2815-HT2931 | Myosin, Light Chain/U02629; Also: HG2815-HT1357 | −1.7970077000 |
| X80343 | p35 regulatory subunit of cdk5 kinase | 1.3159703000 |
| L43579 | (clone 110298)/L43579; Also: L43575 | −1.7886985000 |
| M21005 | Migration inhibitory factor-related protein 8 (MRP8) | −1.7900211000 |
| S74683 | ADP-ribosyltransferase | −1.7919537000 |
| U07000 | BCR (unknown) from breakpoint cluster region (BCR) | −1.7916029000 |
| U10117 | Endothelial-monocyte activating polypeptide II | −1.7917783000 |
| X59417 | PROS-27 | −1.7922165000 |
| *HG2730-HT2828 | Fibrinogen, A Alpha Polypeptide; Also: M58569 | 1.3053514000 |
| X07203 | CD20 receptor (S7) | 1.3053088000 |
| D86956 | KIAA0201 | −1.7865740000 |
| U07151 | GTP binding protein (ARL3) | −1.7839036000 |
| U73167 | H_LUCA146 | −1.7841714000 |
| M76180 | Aromatic amino acid decarboxylase (ddc) | 1.3010300000 |
| Z69923 | DNA sequence from Huntington's Disease Region | 1.3015718000 |
| D80009 | KIAA0187 | 1.2953744000 |
| M34041 | Alpha-2-adrenergic receptor (alpha-2 c2) | −1.7816656000 |
| M96956 | (clone CR-3) teratocarcinoma-derived growth factor 3 TDGF3 | 1.2922561000 |
| M59820 | Granulocyte colony-stimulating factor receptor (CSF3R) | 1.2833012000 |
| HG4258-HT4528 | Kinase Inhibitor P27kip1 Cyclin-Dependent | −1.7678976000 |
| X57129 | H12 histone H1 | 1.2764618000 |
| *HG1595-HT4788 | Heterogeneous Nuclear Ribonucleoprotein I; Also: HG1595-HT4789, | 1.2717876000 |
| | X66975 | |
| M32334 | Intercellular adhesion molecule 2 (ICAM-2) | −1.7634280000 |
| M98343 | Amplaxin (EMS1) | −1.7631471000 |
| X69950 | DNA sequence for Wilms tumor; Also: M60614 | −1.7625848000 |
| AC002450 | BAC clone GS244B22/7q21-q22/AC002450 | 1.2669762000 |
| Z46632 | HSPDE4C1 3,5-cyclic AMP phosphodiesterase | 1.2671717000 |

TABLE 3
Gene targets in MS spinal cord gray matter from a sample characterized by
inflammation by lymphocytes and macrophages, and demyelination.

| Probe set | Gene description | log10 (ratio) fold change |
|---|---|---|
| X05608 | Neurofilament subunit NF-L | −2.9853086000 |
| *M87789 | Anti-hepatitis A IgG V, C, CDR regions; Also: J00221_2 | 3.4707705000 |
| *M63438 | Ig rearranged gamma chain V-J-C region; Also: X96754 | 3.4656016000 |
| L76200 | Guanylate kinase (GUK1) | −2.5051160000 |
| M34516 | Omega light chain protein 14.1 (Ig lambda chain related) | 2.5219506000 |
| S68616 | Na+/H+ exchanger NHE-1 isom | −2.4902746000 |
| L11672 | Kruppel related zinc finger protein (HTF10) | −2.4847446000 |
| L44140 | DNL1L from chromosome X region; Also: X90392 | 2.2062455000 |
| U57341 | Neurofilament triplet L protein/U57341 | −2.4386017000 |
| *X13334 | CD14 myeloid cell-specific leucine-rich glycoprotein | 2.1191968000 |
| M62505 | C5a anaphylatoxin receptor | 2.0890128000 |
| L28821 | Alpha mannosidase II isozyme | −2.3860975000 |
| D50402 | NRAMP1 | 2.0422244000 |
| *X64072 | CD18; Also: M15395 | 2.0330818000 |
| M27826 | Endogenous retroviral protease | −2.3628828000 |
| Z22548 | Thiol-specific antioxidant protein | −2.3496417000 |
| HG3991-HT4261 | Cpg-Enriched Dna, Clone E18 | 2.0112320000 |
| U28488 | Putative G protein-coupled receptor (AZ3B); Also: U62027 | 1.9889735000 |
| M80397 | DNA polymerase delta catalytic subunit; Also: M81735 | −2.3239221000 |
| U09210 | Vesicular acetylcholine transporter | −2.3253617000 |
| J04469 | Mitochondrial creatine kinase (CKMT) | −2.3121244000 |
| U77643 | K12 protein precursor | 1.9392946000 |
| U43916 | Tumor-associated membrane protein homolog (TMP) | 1.9347509000 |
| M27161 | MHC class I CD8 alpha-chain (Leu-2/T8) | −2.2965281000 |
| *X00734 | Beta-tubulin (5-beta) with ten Alu family members | −2.2901738000 |
| D00654 | Enteric smooth muscle gamma-actin | 1.9127421000 |
| HG3286-HT3463 | Crystallin Alpha A | −2.2734643000 |
| L13266 | N-methyl-d-aspartate receptor (NR1-1) | −2.2750809000 |
| D49357 | S-adenosylmethionine synthetase | 1.8856319000 |
| X84213 | BAK BCI-2 homolog; Also: U16811, U23765 | 1.8714562000 |
| U76366 | Treacher Collins syndrome (TCOF1); Also: U84665, U40847 | −2.2445863000 |
| M77829 | Channel-like integral membrane protein (CHIP28); Also: S73482 | 1.8463279000 |
| D63478 | KIAA0144 | −2.2079372000 |
| U93049 | SLP-76 associated protein | 1.8208888000 |
| M36542 | Lymphoid-specific transcription factor; Also: X13810, X13809 | −2.2043574000 |
| HG3731-HT4001 | Ig Heavy Chain, Vdjrc Regions L23566 | 1.8025389000 |
| M95178 | Non-muscle alpha-actinin | 1.7921114000 |
| X98225 | Gastrin-binding protein/X98225 | 1.7825443000 |
| X15306 | NF-H 1 | −2.1689075000 |
| *M24766 | Alpha-2 collagen type IV (COL4A2); Also: X05610 | 1.7779340000 |
| X07834 | Manganese superoxide dismutase SOD2 | 1.7802453000 |
| D44466 | Proteasome subunit p112 | −2.1663931000 |
| Z49254 | L23-related | −2.1637203000 |
| U18550 | GPR3 G protein-coupled receptor | 1.7746994000 |
| J05068 | Transcobalamin I | 1.7700047000 |
| M59820 | Granulocyte colony-stimulating factor receptor (CSF3R) | 1.7714405000 |
| U38480 | Retinoid X receptor-gamma | −2.1610684000 |
| X87212 | Cathepsin C | 1.7590364000 |
| HG2036-HT2090 | Stimulatory Gdp/Gtp Exchange protein C-Ki-Ras P21/Smg P21 | −2.1415673000 |
| Y08409 | Spot14 | −2.1407044000 |
| L47345 | Elongin A | −2.1360464000 |
| U49837 | LIM protein MLP | −2.1346153000 |
| M96944 | B-cell specific TRANSCRIPTION FACTOR (BSAP) | 1.7306208000 |
| M98045 | Folylpolyglutamate synthetase | −2.1298107000 |
| D80004 | KIAA0182 | −2.1272263000 |
| X70991 | MADER | 1.7156601000 |
| X64037 | RNA polymerase II associated protein RAP74 | −2.0997238000 |
| U50360 | Calcium, calmodulin-dependent protein kinase II gamma/U50360 | −2.0863598000 |
| J05257 | Clones MDP4 MDP7 microsomal dipeptidase (MDP) | 1.6624745000 |
| U79261 | Clone 23959; Also: U62317_rna1 | −2.0810321000 |
| X77588 | HG3988-HT4258 and others | −2.0753188000 |
| X17094 | Furin | 1.6512295000 |
| M13207 | Granulocyte-macrophage colony-stimulating factor (CSF1) | −2.0635210000 |
| S85963 | hIRS-1 = rat insulin receptor substrate-1 homolog | 1.6198012000 |
| *X57809 | Rearranged Ig lambda light chain; Also: S42404 | 1.6185978000 |
| U49260 | Mevalonate pyrophosphate decarboxylase (MPD) | −2.0366788000 |
| D42040 | KIAA9001; Also: X62083, M80613 | −2.0292924000 |
| U62293 | LIMK1 (LIM-kinase1); Also: U63721_rna2, U63721_rna2 | −2.0282153000 |
| HG491-HT491 | Fc Receptor lib3 For Igg, Low Affinity | 1.6060050000 |
| HG2797-HT2906 | Clathrin, Light Polypeptide B-Also: X81637, HG2797-HT2905 | −2.0267375000 |
| M25322 | Granule membrane protein-140 | −2.0059844000 |
| X06956 | HALPHA44 alpha-tubulin | −2.0045900000 |
| D29642 | KIAA0053 | 1.5838220000 |
| Y00970 | Acrosin (EC 342110) | 1.5839352000 |
| *S82024 | SCG10 = neuron-specific growth-associated protein | −1.9997722000 |
| D83920 | Uterus ficolin-1 | 1.5799550000 |
| U47621 | Nucleolar autoantigen No55 | 1.5800958000 |
| M85247 | Dopamine D1A receptor/M85247 | −1.9920009000 |
| U37219 | Cyclophilin-like protein CyP-60 | −1.9864918000 |
| **X05299 | (˜95%) major centromere autoantigen CENP-B | 1.5698259000 |
| X63380 | RSRFR2 | 1.5688006000 |
| AF002224 | E6-AP ubiquitin protein ligase 3A/promoter P1 | 1.5625902000 |
| HG2348-HT2444 | Peptide Yy; Also: D13897_rna2 | 1.5661874000 |
| HG2259-HT2348 | Tubulin, Alpha 1; Also: X06956 | −1.9746844000 |
| M36200 | Synaptobrevin 1 (SYB1) | −1.9770946000 |
| M13981 | Inhibin A-subunit | 1.5621968000 |
| D13636 | KIAA0011 | −1.9620140000 |
| U68031 | G protein-coupled receptor (STRL22)/U68031 | 1.5521813000 |
| Z38133 | Myosin; Also: M36769 | 1.5518470000 |
| M80647 | Thromboxane synthase | 1.5441298000 |
| X14474 | Microtubule-associated tau protein | 1.5436335000 |
| X14813 | 3-oxoacyl-CoA thiolase | −1.9496339000 |
| HG1728-HT1734 | CGM1 | 1.5412047000 |
| L35240 | Enigma | 1.5380079000 |
| HG1098-HT1098 | Cystatin D | 1.5345338000 |
| L42176 | (clone 353) DRAL | 1.5358003000 |
| S66431 | RBP2 = retinoblastoma binding protein 2 | 1.5358003000 |
| U58090 | Hs-cul-4A | 1.5331363000 |
| X51954 | UCP1 uncoupling protein/X51954 | 1.5328482000 |
| HG4593-HT4998 | Sodium Channel 1 | −1.9391106000 |
| U17886 | Succinate dehydrogenase iron-protein subunit (sdhB) | −1.9363881000 |
| U37519 | Aldehyde dehydrogenase (ALDH8) | −1.9341827000 |
| D84557 | HsMcm6 | −1.9302484000 |
| M62843 | Antigen in paraneoplastic sensory neuronopathy patients | −1.9305033000 |
| L10377 | (clone CTG-B37) sequence; Also: D38529, U23851, D31840 | 1.5271786000 |
| HG2825-HT2949 | Ret Transforming | −1.9244085000 |
| L10717 | T cell-specific tyrosine kinase | 1.5193028000 |
| M25897 | Platelet factor 4 (PF4) | 1.5222276000 |
| U85265 | Down syndrome critical region 1 (DSCR1) alternative 1 | 1.5216642000 |
| D10995 | Serotonin 1B receptor | −1.9209056000 |
| X52008 | Strychnine binding subunit of inhibitory glycine receptor | −1.9199275000 |
| HG3491-HT3685 | Zinc Finger protein Zfp-36 | 1.5150123000 |
| U20647 | Zinc finger protein (ZNF151) | −1.9093554000 |
| L77213 | Phosphomevalonate kinase | −1.9056610000 |
| S82240 | RhoE = 26 kda GTPase homolog | 1.5021539000 |
| *M74826 | Glutamate decarboxylase (GAD-2) | −1.8953535000 |
| *X07109 | Protein kinase C (PKC) type beta II | −1.8952153000 |
| HG4490-HT4876 | Proline-Rich protein Prb4, Allele | −1.8905607000 |
| HG4051-HT4321 | Choline Acetyltransferase | 1.4882686000 |
| M21665 | Beta-myosin heavy chain; Also: X52889 | 1.4892253000 |
| X81420 | hHKb1 protein | 1.4919523000 |
| L23333 | Corticotropin releasing factor receptor; Also: X72304 | −1.8873359000 |
| D16815 | EAR-1r | 1.4869969000 |
| M25280 | Lymph node homing receptor | 1.4825163000 |
| D43968 | AML1b protein | −1.8803133000 |
| K03183 | Chorionic gonadotropin beta subunit | −1.8803276000 |
| M13232 | Factor VII serine protease precursor; Also: J02933 | −1.8815986000 |
| U23803 | Heterogeneous ribonucleoprotein A0 | −1.8788827000 |
| U59736 | TRANSCRIPTION FACTOR (NFATcb) | −1.8802368000 |
| L09708 | Complement component 2 (C2) allele b | 1.4794162000 |
| X78992 | ERF-2 | 1.4819119000 |
| X80026 | B-cam | −1.8752784000 |
| Y07755 | S100A2 | −1.8752784000 |
| HG2709-HT2805 | Serine/Threonine Kinase | 1.4744348000 |
| L40396 | (clone s22i71) | 1.4763968000 |
| D83784 | KIAA0198 | −1.8699647000 |
| L13972 | Beta-galactoside alpha-23-sialyltransferase (SIAT4A) | −1.8688648000 |
| X16667 | HOX2G from the Hox2 locus | −1.8693784000 |
| U24577 | LDL-phospholipase A2 | 1.4699388000 |
| L41147 | 5-HT6 serotonin receptor | −1.8659918000 |
| U31929 | Orphan nuclear receptor (DAX1); Also: S74720 | −1.8651780000 |
| U79271 | Clones 23920 and 23921 sequence | 1.4633207000 |
| U96131 | HPV16 E1 protein binding protein/U96131 | −1.8599635000 |
| D59253 | NCBP interacting protein 1 | 1.4611983000 |
| J04102 | Erythroblastosis virus onco homolog 2 (ets-2) | 1.4614809000 |
| X68149 | BLR1 Burkitt's lymphoma receptor 1 | 1.4621733000 |
| AF001620 | Trabecular meshwork-induced glucocorticoid response protein | 1.4517097000 |
| S77763 | Nuclear factor erythroid 2 isom f = basic leucine zipper protein | −1.8464144000 |
| L20826 | I-plastin | −1.8419067000 |
| U60415 | bHLH-PAS protein Jap3 | 1.4420092000 |
| X06389 | Synaptophysin (p38) | 1.4336098000 |
| X17360 | HOX 5.1 protein | 1.4333697000 |
| M97496 | Guanylin | 1.4266739000 |
| S81578 | Dioxin-responsive/S81578 | 1.4274050000 |
| U80017 | Survival motor neuron protein | 1.4251371000 |
| X67318 | Procarboxypeptidase A1 | 1.4263486000 |
| M60299 | Alpha-1 collagen type II s 1 2 and 3/M60299 | −1.8246952000 |
| D85939 | p97 homolog | 1.4219328000 |
| M22403 | Blood platelet membrane glycoprotein Ib-alpha (GPIB) | 1.4189234000 |
| X99296 | RD/X99296 | 1.4203205000 |
| M73481 | Gastrin releasing peptide receptor (GRPR) | −1.8188030000 |
| U53476 | Proto-onco Wnt7a | −1.8187206000 |
| D42045 | KIAA0086 | 1.4135512000 |
| M12759 | Ig J chain | 1.4128674000 |
| S71043 | Ig alpha 2 = Ig A heavy chain allotype 2; Also: S55735 | 1.4131120000 |
| X14690 | Plasma inter-alpha-trypsin inhibitor heavy chain H(3) | 1.4075608000 |
| X74039 | Urokinase plasminogen activator receptor | −1.8169866000 |
| X87904 | SEP protein | −1.8142476000 |
| X90840 | Axonal transporter of synaptic vesicles | −1.8153287000 |
| M63256 | Major Yo paraneoplastic antigen (CDR2) | 1.4072771000 |
| U20240 | C/EBP gamma | 1.4061322000 |
| M16707 | Histone H4; clone FO108 | −1.8076683000 |
| X54871 | Ras-related protein Rab5b | 1.3981137000 |
| S72493 | Keratin = keratin 16 homolog; Also: M28439 | −1.8036278000 |
| U68233 | Farnesol receptor HRR-1 (HRR-1) | −1.8026027000 |
| X74794 | P1-Cdc21 | −1.8037791000 |
| X87871 | Hepatocyte nuclear factor 4b; Also: X87870, Z49825 | −1.8050759000 |
| Z84721 | DNA from 16p13.3 Contains alpha and zeta globin | −1.8031156000 |
| M68516 | PCI (plasminogen activator inhibitor 3) from protein C inhibitor | 1.3888114000 |
| U46746 | Dystrobrevin-epsilon; Also: U46744 | 1.3918125000 |
| S68874 | EP3 prostanoid receptor EP3-I; Also: D86096_1, X83858 | −1.8020036000 |
| X89211 | DNA endogenous retroviral like element/X89211 | −1.7982189000 |
| HG3570-HT3773 | Protein Phosphatase Inhibitor Homolog | −1.7935634000 |
| X63755 | High-sulphur keratin | 1.3680604000 |
| L08424 | Achaete scute homologous protein (ASH1) | −1.7847064000 |
| D86062 | KNP-Ib; Also: U53003 | 1.3634666000 |
| M95929 | Homeobox protein (PHOX1) | 1.3627651000 |
| S75313 | MJD1 = MJD1 protein {CAG repeats} | 1.3585874000 |
| X70649 | CI1042 of DEAD box protein family | −1.7784226000 |
| Y00757 | Polypeptide 7B2 | −1.7792698000 |
| D87002 | POM121-like 1 | 1.3552599000 |
| HG2260-HT2349 | Duchenne Muscular Dystrophy protein (Dmd); Also: M18533 | 1.3538201000 |
| M25809 | Endomembrane proton pump subunit | −1.7725967000 |
| X62515 | Basement membrane heparan sulfate proteoglycan | −1.7758834000 |
| U22662 | Nuclear orphan receptor LXR-alpha | 1.3447851000 |
| D79998 | KIAA0176 | −1.7700231000 |
| HG919-HT919 | Dna Polymerase Epsilon Catalytic Subunit | −1.7714956000 |
| J05252 | Also: M95971 | −1.7686381000 |
| U96629 | Hereditary multiple exostosis | 1.3424227000 |
| M74093 | Cyclin | −1.7639892000 |
| S79781 | WT1/S79781 | −1.7641855000 |
| M55024 | Cell surface glycoprotein P3.58;/M55024; Also: M24283 | 1.3357587000 |
| U09550 | Oviductal glycoprotein | 1.3283163000 |
| D26561 | ORF E7 from papillomavirus 5b genome | −1.7600453000 |
| M97252 | Kallmann syndrome (KAL) | 1.3249620000 |
| D17516 | PACAP receptor | 1.3201401000 |
| D49493 | Bone morphogenetic protein-3b | 1.3221159000 |
| L02867 | 62 kDa paraneoplastic antigen | −1.7546350000 |
| M34539 | FK506-binding protein (FKBP) | −1.7572062000 |
| M17183 | Parathyroid hormone-related protein; Also: M24351_3, J03580 | 1.3152354000 |
| D87937 | Alpha(1,2)fucosyltransferase 5 | −1.7500260000 |
| D89377 | MSX-2 | −1.7507012000 |
| U43843 | H-neuro-d4 protein | −1.7503155000 |
| D42054 | KIAA0092 | 1.3057652000 |
| M55905 | Mitochondrial NAD(P)+ dependent malic enzyme | 1.3051620000 |
| U28281 | secretin receptor | −1.7464396000 |
| Z34975 | LDLC | −1.7474409000 |
| HG3187-HT3366 | Tyrosine Phosphatase 1; Also: HG3187-HT3365, U12128 | 1.3023642000 |
| M27543 | Guanine nucleotide-binding protein (Gi) alpha subunit | 1.3007215000 |
| U18991 | Retinal pigment epithelium-specific 61 kDa protein (RPE65) | 1.2986348000 |
| HG4245-HT4515 | Khead Family Afx1 | 1.2941354000 |
| M83652 | Complement component properdin; Also: X57748 | 1.2929203000 |
| HG4234-HT4504 | Methylenetetrahydrofolate Reductase | −1.7398689000 |
| Z74792 | CCAAT transcription binding factor subunit gamma | −1.7387806000 |
| S66896 | Squamous cell carcinoma antigen = serine protease inhibitor | 1.2912578000 |
| D17400 | 6-pyruvoyl-tetrahydropterin synthase | −1.7353993000 |
| Y08134 | ASM-like phosphodiesterase 3b | −1.7374908000 |
| D29992 | Placental protein 5 (PP5) | 1.2797753000 |
| X15088 | GNAT1 transducin alpha-chain | 1.2820442000 |
| HG1747-HT1764 | Proto-Oncogene Met; Also: J02958, U08818 | −1.7316895000 |
| M12886 | T-cell receptor active beta-chain | −1.7301764000 |
| U07231 | G-rich sequence factor-1 (GRSF-1) | −1.7299743000 |
| X17254 | TRANSCRIPTION FACTOR Eryf1 | −1.7314880000 |
| X77567 | InsP3 5-phosphatase; Also: Z31695 | 1.2761490000 |
| V00535 | IFNB 1; Also: J00218_rna1, V00547, M28622, V00534_rna1 | 1.2694863000 |
| L22454 | Nuclear respiratory factor-1 (NRF-1) | −1.7246854000 |
| M18700 | D00306, M16630, M18692 | −1.7273379000 |
| U79245 | Clone 23586 sequence | −1.7266253000 |
| X02404 | CGRP from medullary thyroid carcinoma (MTC) | −1.7271344000 |
| Z46376 | HK2 hexokinase II | 1.2635177000 |
| D14134 | RAD51 | 1.2598327000 |
| U79293 | Clone 23948 sequence | 1.2617385000 |
| L21998 | Intestinal mucin (MUC2) | −1.7202627000 |
| M65062 | Insulin-like growth factor binding protein 5 (IGFBP-5) | 1.2530956000 |
| U78095 | Placental bikunin | −1.7129652000 |
| D10495 | Protein kinase C delta-type | 1.2435668000 |
| HG3288-HT3465 | Xanthine Dehydrogenase | −1.7119126000 |
| M21984 | (clone PWHTnT16) skeletal muscle Troponin T | −1.7104347000 |
| U47928 | Protein A alternatively spliced m 2 (A-2) | −1.7110687000 |
| X52228 | Secreted epithelial tumor mucin antigen | −1.7084421000 |
| X59373 | HOX4D a homeobox protein | −1.7115964000 |
| HG2841-HT2969 | Albumin, 3; Also: HG2841-HT2970, HG2841-HT2968 | 1.2377036000 |
| U29589 | m3 muscarinic acetylcholine receptor (CHRM3) | 1.2402996000 |

TABLE 4
Gene targets in MS spinal cord gray matter from a sample with axonal loss.

| Probe set | Gene description | log10 (ratio) fold change |
|---|---|---|
| D26129 | RNase A | −2.7780879000 |
| *M87789 | Anti-hepatitis A IgG V, C, CDR regions; Also: J00221_2 | 3.7091911000 |
| *M63438 | Ig rearranged gamma chain, V-J-C region; Also: X96754 | 3.6640039000 |
| L13210 | Mac-2 binding protein | −2.6950437000 |
| L16862 | G protein-coupled receptor kinase (GRK6) | −2.6041720000 |
| M25280 | Lymph node homing receptor | 2.0987648000 |
| D49357 | S-adenosylmethionine synthetase | 2.0759381000 |
| L76200 | Guanylate kinase (GUK1) | −2.5051160000 |
| X74570 | Gal-beta(1-3/1-4)GlcNAc alpha-23-sialyltransferase | −2.4956310000 |
| S68616 | Na+/H+ exchanger NHE-1 isom | −2.4902746000 |
| L44140 | DNL1L from chromosome X region; Also: X90392 | 2.0235817000 |
| L14565 | Peripherin (PRPH) s 1-9 | −2.4614422000 |
| S76638 | p50-NF-kappa B homolog | 1.9947998000 |
| HG2709-HT2805 | Serine/Threonine Kinase | 1.9764875000 |
| X84213 | BAK BCI-2 homolog; Also: U16811, U23765 | 1.9659539000 |
| *X63578 | Parvalbumin | −2.3866104000 |
| X14474 | Microtubule-associated tau protein | 1.9417846000 |
| *D90086 | Pyruvate dehydrogenase beta subunit | −2.3701197000 |
| L41143 | Expressed pseudo TCTA at t(1;3) translocation site | −2.3607117000 |
| M63573 | Secreted cyclophilin-like protein (SCYLP) | 1.9226736000 |
| *X64072 | CD18; Also: M15395 | 1.9213222000 |
| Z22548 | Thiol-specific antioxidant protein | −2.3496417000 |
| M72885 | GOS2; Also: M69199_rna1 | 1.9171115000 |
| HG1067-HT1067 | Mucin/M22406 | 1.9114934000 |
| M80397 | DNA polymerase delta catalytic subunit; Also: M81735 | −2.3239221000 |
| M22632 | Mitochondrial aspartate aminotransferase | −2.3186893000 |
| *X00734 | Beta-tubulin (5-beta) with ten Alu family members | −2.2901738000 |
| J04501 | Muscle glycogen synthase | 1.8619822000 |
| L13266 | N-methyl-d-aspartate receptor (NR1-1) | −2.2750809000 |
| D16583 | L-histidine decarboxylase | 1.8302678000 |
| X87212 | Cathepsin C | 1.8308174000 |
| U76366 | Treacher Collins syndrome (TCOF1); Also: U84665, U40847 | −2.2445863000 |
| X69433 | Mitochondrial isocitrate dehydrogenase (NADP+) | −2.2474823000 |
| Z29331 | (23k/3) ubiquitin-conjugating enzyme UbcH2 | 1.8159097000 |
| D63487 | KIAA0153 | −2.2291697000 |
| U73799 | Dynactin/U73799 | 1.7998573000 |
| D12625 | Neurofibromin | 1.7918309000 |
| D82348 | Aminoimidazole carboxamide ribo-nt transmylase/inosinicase | −2.2076344000 |
| M36542 | Lymphoid-specific transcription factor; Also: X13810, X13809 | −2.2043574000 |
| M21665 | Beta-myosin heavy chain; Also: X52889 | 1.7723363000 |
| U89336 | Notch 4 | −2.1940632000 |
| J04132 | T cell receptor zeta-chain | 1.7635777000 |
| U79271 | Clones 23920 and 23921 sequence | 1.7502416000 |
| AC000099 | Cosmid g0771a003 | −2.1675758000 |
| D90276 | CGM7 nonspecific cross-reacting antigen (NCA) | −2.1670957000 |
| HG3995-HT4265 | Cpg-Enriched Dna Clone S19 | −2.1596800000 |
| AF002224 | E6-AP ubiquitin protein ligase 3A/promoter P1 | 1.7346798000 |
| X67325 | p27 | −2.1546522000 |
| L32137 | Germline oligomeric matrix protein (COMP) | 1.7239480000 |
| Z46632 | HSPDE4C1 3,5-cyclic AMP phosphodiesterase | 1.7264012000 |
| Y08409 | Spot14 | −2.1407044000 |
| U56976 | Calmodulin dependent phosphodiesterase PDE1B1 | 1.7128601000 |
| X04145 | T-cell receptor T3 gamma polypeptide | 1.7165042000 |
| M13755 | Interferon-induced 17-kDa/15-kDa protein | −2.1362051000 |
| S66431 | RBP2 = retinoblastoma binding protein 2 | 1.7037212000 |
| M98045 | Folylpolyglutamate synthetase | −2.1298107000 |
| U47621 | Nucleolar autoantigen No55 | 1.6981708000 |
| X57351 | 1-8D from interferon-inducible family | −2.1186367000 |
| HG491-HT491 | Fc Receptor lib3 For lgg, Low Affinity | 1.6904619000 |
| X89986 | NBK apoptotic inducer protein; Also: U49730, U34584 | 1.6868596000 |
| HG3987-HT4257 | Cpg-Enriched Dna Clone E06 | −2.1094182000 |
| AD000684 | LISCH7 (liver-specific bHLH-Zip transcription factor) | 1.6814222000 |
| **X05299 | (˜95%) major centromere autoantigen CENP-B | 1.6791289000 |
| AF001294 | IPL | −2.1022192000 |
| U50743 | NaK-ATPase gamma subunit | −2.0976043000 |
| M65085 | Follicle stimulating hormone receptor | 1.6673862000 |
| M35252 | CO-029 | −2.0848443000 |
| L36463 | Ras inhibitor (Rin1) | 1.6544690000 |
| U79261 | Clone 23959; Also: U62317_rna1 | −2.0810321000 |
| X99142 | Hair keratin hHb6 | −2.0812573000 |
| HG3991-HT4261 | Cpg-Enriched Dna, Clone E18 | 1.6428106000 |
| U13369 | Ribosomal DNA repeating unit/U13369 | 1.6399345000 |
| U46746 | Dystrobrevin-epsilon; Also: U46744 | 1.6422686000 |
| M13207 | Granulocyte-macrophage colony-stimulating factor (CSF1) | −2.0635210000 |
| X69838 | G9a | −2.0635210000 |
| Z22555 | Encoding CLA-1 | −2.0576186000 |
| Z33905 | 43 kD acetylcholine receptor-associated protein (Rapsyn) | 1.6197193000 |
| U39573 | Salivary peroxidase | −2.0552349000 |
| J03600 | Lipoxygenase | −2.0488301000 |
| Y00757 | Polypeptide 7B2 | −2.0487933000 |
| L13698 | Growth-arrest-specific protein (gas) | 1.6115584000 |
| U22662 | Nuclear orphan receptor LXR-alpha | 1.6076694000 |
| X17360 | HOX 5.1 protein | 1.6118826000 |
| Z38133 | Myosin; Also: M36769 | 1.6092717000 |
| X70991 | MADER | 1.6063349000 |
| M13981 | Inhibin A-subunit | 1.6011419000 |
| U49260 | Mevalonate pyrophosphate decarboxylase (MPD) | −2.0366788000 |
| AF009674 | Axin | 1.5857071000 |
| U91316 | Acyl-CoA thioester hydrolase | −2.0287237000 |
| D86971 | KIAA0217 | 1.5813868000 |
| HG2797-HT2906 | Clathrin, Light Polypeptide B- Also: X81637, HG2797-HT2905 | −2.0267375000 |
| M16707 | Histone H4; clone FO108. | −2.0251522000 |
| D50923 | KIAA0133 | 1.5725231000 |
| S69369 | PAX3A = TRANSCRIPTION FACTOR | 1.5694910000 |
| M63582 | Preprothyrotropin-releasing hormone | −2.0136797000 |
| D49493 | Bone morphogenetic protein-3b | 1.5591882000 |
| AC002115 | COX6B | −2.0121515000 |
| X98833 | Zinc finger protein, Hsal1 | −2.0089194000 |
| D50532 | Macrophage lectin 2 | 1.5516018000 |
| D50913 | KIAA0123 | −2.0061450000 |
| M25322 | Granule membrane protein-140 | −2.0059844000 |
| M33882 | p78 protein | −2.0057702000 |
| X06956 | HALPHA44 alpha-tubulin | −2.0045900000 |
| M64231 | Spermidine synthase | −1.9982048000 |
| HG3748-HT4018 | Basic Transcription Factor 44 Kda Subunit | 1.5370001000 |
| K03021 | Tissue plasminogen activator (PLAT) | −1.9892829000 |
| Z14244 | CoxVIIb cytochrome c oxidase subunit VIIb | −1.9903944000 |
| U37219 | cyclophilin-like protein CyP-60 | −1.9864918000 |
| D50402 | NRAMP1 | 1.5099211000 |
| HG2259-HT2348 | Tubulin, Alpha 1; Also: X06956 | −1.9746844000 |
| HG2463-HT2559 | Guanine Nucleotide-Binding protein G25k | −1.9741085000 |
| X04201 | Skeletal muscle tropomyosin | −1.9737627000 |
| X97630 | Serine threonine protein kinase EMK | −1.9752020000 |
| J00220 | IGHA1 from Ig germline H-chain G-E-A region A: gamma-3 5 | 1.4976974000 |
| L76568 | S26 from excision and cross link repair protein ERCC4/L76568 | −1.9693577000 |
| M96944 | B-cell specific TRANSCRIPTION FACTOR (BSAP) | 1.4969988000 |
| U01828 | Microtubule-associated protein 2 (MAP2) | 1.4951973000 |
| U96629 | Hereditary multiple exostosis | 1.4901693000 |
| M60298 | Erythrocyte membrane protein band 42 (EPB42) | 1.4847268000 |
| *U89606 | Pyridoxal kinase | 1.4845845000 |
| U22970 | 16-Jun (interferon-inducible peptide precursor) | −1.9576671000 |
| U01157 | Glucagon-like peptide-1 receptor with CA dinucleotide repeat | 1.4775752000 |
| X16282 | Zinc finger protein (clone 647) | 1.4782778000 |
| X81851 | IL-4 splice variant/X81851; Also: M13982 | 1.4752438000 |
| U25988 | Pregnancy-specific glycoprotein 13 (PSG1) | −1.9489628000 |
| X71490 | Vacuolar proton ATPase subunit D | −1.9519443000 |
| X05908 | Lipocortin | 1.4691078000 |
| D86956 | KIAA0201 | 1.4661771000 |
| X13589 | Aromatase (estrogen synthetase) | 1.4643405000 |
| Z14093 | Branched chain decarboxylase alpha subunit | 1.4654723000 |
| *M85220 | Heavy chain disease IgA chain CH3 region | 1.4526297000 |
| U02031 | Sterol regulatory element binding protein-2 | 1.4533794000 |
| L21993 | Adenylyl cyclase | 1.4503590000 |
| D84454 | UDP-galactose translocator | 1.4436628000 |
| U20240 | C/EBP gamma | 1.4431197000 |
| D84557 | HsMcm6 | −1.9302484000 |
| M62843 | Antigen of paraneoplastic sensory neuronopathy patients | −1.9305033000 |
| U65676 | Hermansky-Pudlak syndrome protein (HPS) | 1.4405943000 |
| Z22780 | Cylicin | 1.4387218000 |
| X89101 | Fas (Apo-1, CD95)/X89101; Also: X83493, X63717, X83492 | −1.9231144000 |
| L25444 | TAFII70-alpha | −1.9207102000 |
| X52008 | Strychnine binding subunit of inhibitory glycine receptor | −1.9199275000 |
| *M58459 | Ribosomal protein (RPS4Y) isom | −1.9165197000 |
| U78525 | Eukaryotic translation initiation factor (elF3) | −1.9140786000 |
| X52611 | Transcription factor AP-2; Also: HG2465-HT4871, M36711 | −1.9158613000 |
| U60415 | bHLH-PAS protein Jap3 | 1.4213571000 |
| AF000545 | Putative purinergic receptor P2Y10 | −1.9043097000 |
| *M35999 | Platelet glycoprotein IIIa (GPIIIa) | 1.4089180000 |
| X07743 | Pleckstrin (P47) | 1.4044376000 |
| X56687 | Autoantigen NOR-90; Also: X53461, X53390, U65487_rna1 | −1.8983823000 |
| M87507 | Interleukin-1 beta convertase (IL1BCE); Also: U13697 | 1.4005185000 |
| U83598 | Death domain receptor 3 soluble form (DDR3); Also: U94512 | −1.8947313000 |
| *X07109 | Protein kinase C (PKC) type beta II | −1.8952153000 |
| M76446 | Alpha-A1-adrenergic receptor | 1.3928727000 |
| D38498 | PMS5 (yeast PMS1 homolog) | −1.8904909000 |
| U64998 | Ribonuclease k6 precursor/U64998 | 1.3909219000 |
| U91327 | Chromosome 12p15 BAG clone CIT987SK-99D8 sequence | 1.3911998000 |
| U18235 | ATP-binding cassette protein (ABC2) HFBCD04 clone | 1.3866834000 |
| U18919 | Chromosome 17q12-21 clone pOV-2 | 1.3860456000 |
| L19183 | MAC30 | −1.8868431000 |
| D43968 | AML1b protein | −1.8803133000 |
| M94065 | Dihydroorotate dehydrogenase | −1.8800272000 |
| U38268 | Cytochrome b pseudo/U38268 | −1.8799556000 |
| U61262 | Neogenin | −1.8810992000 |
| U87964 | Putative G-protein (GP-1) | −1.8814560000 |
| L48692 | (clone p5-23-3) | 1.3767594000 |
| HG167-HT167 | Hypothetical protein Npiiy20/M76676 | 1.3646448000 |
| J04449 | (clone NF 10) cytochrome P-450 nifedipine oxidase | 1.3637999000 |
| D83784 | KIAA0198 | −1.8699647000 |
| X16667 | HOX2G from the Hox2 locus | −1.8693784000 |
| HG2755-HT2862 | T-Plastin | −1.8637688000 |
| U96094 | Sarcolipin (SLN) | −1.8651040000 |
| X01038 | Fetal apolipoprotein AI precursor; Also: X07496, X00566 | −1.8629658000 |
| X77794 | Cyclin G1 | −1.8655481000 |
| HG2992-HT5186 | Beta-Hexosaminidase Alpha Polypeptide | 1.3552284000 |
| L09708 | Complement component 2 (C2) allele b | 1.3547290000 |
| M24486 | Prolyl 4-hydroxylase alpha subunit; Also: M24487, U14620_1 | 1.3536952000 |
| U27699 | PephBGT-1 betaine-GABA transporter | 1.3549090000 |
| Z34974 | Plakophilin; Also: X79293 | 1.3510229000 |
| D82061 | Short-chain alcohol dehydrogenase family | −1.8595886000 |
| D84361 | p52 and p64 N-Shc | −1.8598285000 |
| U64197 | Chemokine exodus | −1.8589881000 |
| M58460 | 75-kD autoantigen (PM-Sc1) | 1.3408405000 |
| U28749 | High-mobility group phosphoprotein isoform I-C (HMGIC) | 1.3392448000 |
| U49973 | Tigger 1 transposable element | 1.3372595000 |
| *D84145 | WS-3 | −1.8500333000 |
| HG982-HT982 | Pre-T/Nk-Cell-Associated protein 1f6; Also: L17326 | −1.8476498000 |
| X62078 | GM2 activator protein | −1.8455631000 |
| M19684 | Alpha-1-antitrypsin-related protein | 1.3239736000 |
| U51003 | DLX-2 (DIx2); Also: L07919 | 1.3250466000 |
| M15169 | Beta-2-adrenergic receptor | 1.3215725000 |
| M22403 | Blood platelet membrane glycoprotein lb-alpha (GPIB) | 1.3166020000 |
| U43916 | Tumor-associated membrane protein homolog (TMP) | 1.3161801000 |
| D16581 | 8-oxo-dGTPase | −1.8333200000 |
| U00946 | Clone A9A2BRB5 (CAC)n/(GTG)n repeat-containing | 1.3119428000 |
| *U62317 | Hypothetical protein 384D8_7 | 1.3095598000 |
| HG2707-HT2803 | Serine/Threonine Kinase | 1.3074800000 |
| L75847 | Zinc finger protein 45 (ZNF45) | 1.3063484000 |
| AF001548 | 815A9.1 myosin heavy chain from chromosome 16 BAC | −1.8301883000 |
| M59916 | Acid sphingomyelinase (ASM) | −1.8284988000 |
| S75578 | 4-aminobutyrate aminotransferase/S75578; Also: L32961 | −1.8297861000 |
| M83822 | Beige-like protein (BGL) | 1.3001886000 |
| M96132 | MHC class II HLA-DR-beta-1*09012 (HLA-DRB1*09012) | 1.3008128000 |
| J02943 | Corticosteroid binding globulin | −1.8248577000 |
| M60299 | Alpha-1 collagen type II s 1 2 and 3/M60299 | −1.8246952000 |
| HG3088-HT3263 | Splicing Factor Sc35 m 3 | 1.2950756000 |
| X78925 | HZF2 zinc finger protein | 1.2900346000 |
| M73481 | Gastrin releasing peptide receptor (GRPR) | −1.8188030000 |
| L11931 | Cytosolic serine hydroxymethyltransferase (SHMT) | 1.2870175000 |
| X60487 | H4/h H4 histone | 1.2873538000 |
| U38964 | PMS2 related (hPMSR2); Also: D38502 | −1.8154117000 |
| M17183 | Parathyroid hormone-related protein; Also: M24351_3, J03580 | 1.2785250000 |
| D85433 | MURR1 | −1.8097280000 |
| Z48481 | Membrane type matrix metalloproteinase 1 | −1.8082110000 |
| M87860 | S-lac lectin L-14-II (LGALS2) | 1.2683541000 |
| L32866 | Effector cell protease receptor-1 (EPR-1) | −1.8044802000 |
| M11726 | Pancreatic polypeptide | −1.8053309000 |
| Z84721 | DNA from 16p13.3 Contains alpha and zeta globin | −1.8031156000 |
| S58733 | pp52 = B lymphocyte signal transduction | 1.2642308000 |
| D14446 | HFREP-1 | −1.7990820000 |
| D37931 | RNase4 | −1.8018323000 |
| *M11749 | Thy-1 glycoprotein | −1.7987879000 |
| U01120 | Glucose-6-phosphatase | −1.7976137000 |
| HG3570-HT3773 | Protein Phosphatase Inhibitor Homolog | −1.7935634000 |
| Z72499 | Herpesvirus associated ubiquitin-specific protease (HAUSP) | −1.7969211000 |
| U28488 | Putative G protein-coupled receptor (AZ3B); Also: U62027 | 1.2483725000 |
| U20860 | Angiotensin II type 2 receptor | 1.2451424000 |
| L00205 | K6b epidermal keratin type II | −1.7875490000 |
| S74683 | ADP-ribosyltransferase | −1.7919537000 |
| U63336 | MHC Class I region proline rich protein | −1.7902852000 |
| HG896-HT896 | Thrombospondin 2 | 1.2414220000 |
| M27878 | DNA binding protein (HPF2) | 1.2395497000 |
| U39226 | Myosin VIIA (USH1B) | 1.2376463000 |
| X79510 | Protein-tyrosine-phosphatase D1 | 1.2361010000 |
| L08424 | Achaete scute homologous protein (ASH1) | −1.7847064000 |
| HG2614-HT2710 | Collagen Type Viii Alpha 1 | 1.2321764000 |
| Z70218 | MN1 protein (clone ICRFp507I0498); Also: X82209 | 1.2278215000 |
| U59228 | Ectodermal dysplasia protein (EDA) | −1.7784226000 |
| U44848 | Nuclear respiratory factor 1 (NRF1)/U44848/ | 1.2209368000 |
| HG3242-HT3419 | Calcium Channel, Voltage-Gated, Alpha 1e Subunit, 2 | −1.7753767000 |
| M25809 | Endomembrane proton pump subunit | −1.7725967000 |
| M27318 | Interferon (IFN-alpha-M1) | 1.2139137000 |
| X51757 | Heat-shock protein HSP70B′ | 1.2104790000 |
| X99350 | HFH4 | 1.2102298000 |
| Y08564 | GalNAc-T4/Y08564 | 1.2076344000 |
| HG4258-HT4528 | Kinase Inhibitor P27kip1 Cyclin-Dependent | −1.7678976000 |
| HG919-HT919 | Dna Polymerase Epsilon Catalytic Subunit | −1.7714956000 |
| D83920 | Uterus ficolin-1 | 1.2042557000 |
| M62505 | C5a anaphylatoxin receptor | 1.2027830000 |
| Y08766 | Splicing factor, SF1-Bo isoform; Also: L49380 | 1.2049504000 |
| M32334 | Intercellular adhesion molecule 2 (ICAM-2) | −1.7634280000 |
| S79781 | WT1/S79781 | −1.7641855000 |
| X54380 | Pregnancy zone protein | 1.1975562000 |

TABLE 5
Gene targets in MS spinal cord white matter from a sample with minimal to no inflammation.

| Probe set | Gene description | log10 (ratio) fold change |
|---|---|---|
| M84739 | Autoantigen calreticulin | 2.9768541000 |
| M13755 | Interferon-induced 17-kDa/15-kDa protein | −2.6319001000 |
| M29194 | Triglyceride lipase | 2.7169627000 |
| M77829 | Channel-like integral membrane protein (CHIP28); Also: S73482 | 2.6933453000 |
| AF001359 | Mismatch repair protein (hMLH1)/AF001359 | −2.4844422000 |
| X95876 | G-protein coupled receptor | −2.4699324000 |
| AB000896 | Cadherin FIB2 | −2.4188397000 |
| L48513 | Paraoxonase 2 (PON2) | −2.4034637000 |
| M21904 | 4F2 glycosylated heavy chain (4F2HC) antigen | 2.5422028000 |
| D49818 | Fructose 6-phosphate 2-kinase/fructose 2 6-bisphosphatase | 2.5278232000 |
| X00734 | Beta-tubulin (5-beta) with ten Alu family members | 2.5225746000 |
| ***D16480 | Mitochondrial enoylCoA hydratase | 2.4726833000 |
| M96326 | Azurocidin | −2.3481101000 |
| U35234 | Protein tyrosine phosphatase sigma | 2.4405943000 |
| M65066 | cAMP-dependent protein kinase regulatory subunit RI-beta | −2.3362095000 |
| X86401 | L-arginine: glycine amidinotransferase | 2.4148938000 |
| AB000895 | Cadherin FIB1 | −2.3181155000 |
| D87433 | KIAA0246 | −2.3191061000 |
| U45328 | Ubiquitin-conjugating enzyme (UBE2I); Also: U31882, U66867 | 2.3617749000 |
| D50863 | TESK1 | −2.2965007000 |
| *D61391 | Phosphoribosypyrophosphate synthetase-associated protein 39 | −2.2951821000 |
| HG3945-HT4215 | Phospholipid Transfer protein | 2.3041673000 |
| AD000092 | RAD23A homolog | −2.2698630000 |
| M60299 | Alpha-1 collagen type II s 1 2 and 3/M60299 | 2.2705624000 |
| U37219 | Cyclophilin-like protein CyP-60 | −2.2589364000 |
| M90299 | Glucokinase (GCK) | −2.2545481000 |
| L24774 | Delta3, delta2-CoA-isomerase; Also: Z25821_rna1 Z25820 | 2.2454882000 |
| D43968 | AML 1b protein | −2.2463139000 |
| M63573 | Secreted cyclophilin-like protein (SCYLP) | 2.2362475000 |
| U12707 | Wiskott-Aldrich syndrome protein (WASP); Also: U19927 | 2.2370408000 |
| D63486 | KIAA0152 | 2.2290416000 |
| D15049 | Protein tyrosine phosphatase | −2.2322971000 |
| M96684 | Pur (pur-alpha) | 2.2042557000 |
| J02947 | Extracellular-superoxide dismutase (SOD3) | 2.1949304000 |
| L05148 | Protein tyrosine kinase related sequence | −2.2048658000 |
| M36429 | Transducin beta-2 subunit; Also: M16538 | 2.1834122000 |
| U66559 | Anaplastic lymphoma kinase receptor | −2.1918002000 |
| X17651 | Myf-4 myogenic determination factor | −2.1869563000 |
| K03189 | Chorionic gonadotropin beta subunit | 2.1595672000 |
| L41067 | NF-AT4c | −2.1758016000 |
| J04501 | Muscle glycogen synthase | 2.1481347000 |
| S78085 | PDCD2 = programmed cell death-2/Rp8 homolog | −2.1691599000 |
| X81372 | Biphenyl hydrolase-related protein | −2.1664301000 |
| U03270 | Centrin | −2.1609185000 |
| U24183 | Phosphofructokinase (PFKM); Also: HG1849-HT1878 | 2.1302939000 |
| J00268 | Insulin; Also: V00565, X70508, L15440, M10039 | −2.1558672000 |
| D28423 | Pre-splicing factor SRp20 | 2.1225435000 |
| L76702 | B56-delta | 2.1264561000 |
| M91585 | Br140 | 2.1259690000 |
| M57609 | DNA-binding protein (GLI3) | −2.1494501000 |
| X15331 | Phosphoribosylpyrophosphate synthetase subunit one | 2.1215598000 |
| L00205 | K6b epidermal keratin type II | −2.1467480000 |
| L43579 | (clone 110298)/L43579; Also: L43575 | 2.1151110000 |
| D49490 | Disulfide isomerase-related protein | −2.1380657000 |
| M31776 | M25296 and others | 2.1119343000 |
| U23430 | Cholecystokinin type A receptor (CCK-A); Also: L19315 | 2.1105897000 |
| X04366 | Calcium activated neutral protease large subunit (muCANP) | −2.1371166000 |
| D80003 | KIAA0181 | −2.1313780000 |
| D14822 | CBFA2T1 | −2.1245042000 |
| U82671 | HSP1-A from cosmids from Xq28 | 2.0900816000 |
| U40002 | Hormone-sensitive lipase testicular isoform; Also: L11706 | 2.0870323000 |
| Z48314 | Apomucin; Also: U06711 | 2.0870712000 |
| HG3242-HT4231 | Calcium Channel, Voltage-Gated, Alpha 1e Subunit, 3 | −2.1080574000 |
| D30755 | KIAA0113 | −2.1044871000 |
| U77968 | Neuronal PAS1 (NPAS1) | 2.0672569000 |
| J02645 | Translational initiation factor (elF-2) alpha subunit | −2.0896402000 |
| U49278 | Putative DNA-binding protein | −2.0898168000 |
| L32866 | Effector cell protease receptor-1 (EPR-1) | −2.0857364000 |
| X13810 | OTF-2 lymphoid-specific transcription factor; Also: M36542 | −2.0805363000 |
| U15131 | p126(ST5) | 2.0398106000 |
| D11094 | MSS1 | 2.0364293000 |
| AB000409 | MNK1 | −2.0695756000 |
| S81737 | Alpha 1 syntrophin; Also: U40571 | 2.0324173000 |
| M58026 | NB-1 | 2.0184925000 |
| X13956 | 12S RNA induced by poly(rI) poly(rC) and Newcastle virus | −2.0547088000 |
| D28532 | Renal Na+-dependent phosphate cotransporter | −2.0486360000 |
| X99142 | Hair keratin hHb6 | 2.0111474000 |
| U15655 | Ets domain prot ERF | 2.0045363000 |
| U16031 | TRANSCRIPTION FACTOR IL-4 Stat | −2.0447357000 |
| U78793 | Folate receptor alpha (hFR)/U78793 | −2.0437551000 |
| HG4757-HT5207 | Oncogene MII-Af4, Fusion Activated; Also: L13773 | −2.0424771000 |
| L38503 | Glutathione S-transferase theta 2 (GSTT2) | −2.0343276000 |
| D13988 | Rab GDI | 1.9896722000 |
| M96738 | Somatostatin receptor subtype 3 (SSTR3); Also: Z86000 | 1.9881128000 |
| U86759 | Netrin-2 like protein (NTN2I); Also: U86758_rna1 | 1.9829493000 |
| D84239 | IgG Fc binding protein | −2.0197392000 |
| AF002224 | E6-AP ubiquitin protein ligase 3A/promoter P1 | 1.9738203000 |
| *M94250 | Retinoic acid inducible factor (MK) | 1.9708116000 |
| X68688 | ZNF33B; Also: D31763 | −2.0095571000 |
| M91083 | DNA-binding protein (HRC1) | 1.9637878000 |
| L41143 | Expressed pseudo TCTA at t(1;3) translocation site | 1.9602033000 |
| U08191 | R kappa B; Also: X80878 | 1.9614211000 |
| X54637 | Tyk2 non-receptor protein tyrosine kinase | 1.9566486000 |
| D79998 | KIAA0176 | −1.9967305000 |
| U71364 | Serine protase inhibitor (P19) | −1.9973864000 |
| M32879 | Steroid 11-beta-hydroxylase (CYP11B1) | 1.9385197000 |
| *U37408 | CtBP | 1.9400182000 |
| X02958 | Interferon alpha IFN-alpha 6 | −1.9815921000 |
| M15881 | Uromodulin (Tamm-Horsfall glycoprotein) | −1.9748570000 |
| U85658 | TRANSCRIPTION FACTOR ERF-1 | −1.9673139000 |
| X12517 | U1 small nuclear RNP-specific C protein | 1.9188164000 |
| U78180 | Sodium channel 2 (hBNaC2) | −1.9604708000 |
| M21984 | (clone PWHTnT16) skeletal muscle Troponin T | −1.9555675000 |
| X16260 | Inter-alpha-trypsin inhibitor subunit 3; Also: X69532_rna1 | 1.9103576000 |
| X63380 | RSRFR2 | 1.9033593000 |
| M21389 | Keratin type II (58 kD) | −1.9454686000 |
| U07919 | Aldehyde dehydrogenase 6 | −1.9452223000 |
| X76942 | U41740 and others | −1.9463294000 |
| X89101 | Fas (Apo-1, CD95)/X89101; Also: X83493, X63717, X83492 | −1.9459607000 |
| J05582 | Pancreatic mucin; Also: J05581 | 1.9006585000 |
| M22919 | Non-muscle myosin light chain MLC | −1.9378939000 |
| L08488 | Inositol polyphosphate 1-phosphatase | 1.8954482000 |
| U68723 | Checkpoint suppressor 1 | 1.8934843000 |
| X82850 | Thyroid transcript factor 1; Also: U43203, U33749 | 1.8945929000 |
| M59916 | Acid sphingomyelinase (ASM) | 1.8904865000 |
| X07315 | PP15 (placental protein 15) | 1.8901415000 |
| X58298 | lnterleukin-6-receptor; Also: M20566 | 1.8918161000 |
| U37519 | Aldehyde dehydrogenase (ALDH8) | −1.9301847000 |
| HG3884-HT4154 | Homeotic protein Hpx-42 | 1.8873359000 |
| **X05299 | (˜95%) major centromere autoantigen CENP-B | 1.8790959000 |
| X13293 | B-myb | 1.8782345000 |
| D79985 | KIAA0163 | −1.9194703000 |
| J00220 | IGHA1 from Ig germline H-chain G-E-A region A: gamma-3 5 | 1.8759989000 |
| J03171 | Interferon-alpha receptor (HuIFN-alpha-Rec) | −1.9122221000 |
| U44754 | PSE-binding factor PTF gamma subunit | −1.9075457000 |
| U53442 | p38Beta MAP kinase | −1.9100905000 |
| *X64072 | CD18; Also: M15395 | 1.8668778000 |
| M15465 | Pyruvate kinase type L; Also: D13243 | −1.9063350000 |
| X87870 | Hepatocyte nuclear factor 4a | −1.9037680000 |
| L43338 | (clone JJ1a) cadherin/L43338 | −1.8995469000 |
| Z19002 | PLZF kruppel-like zinc finger protein | −1.8955607000 |
| U89896 | Casein kinase I gamma 2 | 1.8524800000 |
| D45917 | TIMP-3; Also: U14394 | −1.8909796000 |
| U06698 | Neuronal kinesin heavy chain | −1.8904210000 |
| L02867 | 62 kDa paraneoplastic antigen | 1.8457180000 |
| M21302 | Small proline rich protein (sprll), clone 174 N | 1.8464883000 |
| U60116 | Skeletal muscle LIM-protein SLIM2 | 1.8438554000 |
| X81420 | hHKb1 protein | 1.8452505000 |
| AB002315 | KIAA0317 | −1.8874766000 |
| D86971 | KIAA0217 | 1.8337844000 |
| L31881 | Nuclear factor I-X | 1.8360075000 |
| X59303 | G7a valyl-tRNA synthetase; Also: M98326 | 1.8369567000 |
| U65011 | Preferentially expressed antigen of melanoma (PRAME) | −1.8787920000 |
| Z71389 | Skin-antimicrobial-peptide 1 (SAP1)/Z71389 | −1.8800987000 |
| U15932 | Dual-specificity protein phosphatase | −1.8727388000 |
| X13766 | Beta-casein; Also: L10615, X17070 | −1.8754953000 |
| X73079 | Encoding Polymeric Ig receptor | −1.8757845000 |
| U50360 | Calcium, calmodulin-dependent protein kinase II gamma/U50360 | 1.8249558000 |
| AF009674 | Axin | −1.8701112000 |
| M58297 | Zinc finger protein 42 (MZF-1) | −1.8720105000 |
| U65093 | Msg1-related 1 (mrg1) | −1.8704039000 |
| U83192 | Post-synaptic density protein 95 (PSD95) | 1.8185558000 |
| J05614 | Proliferating cell nuclear antigen (PCNA) promoter region | 1.8165727000 |
| *M63438 | Ig rearranged gamma chain , V-J-C region; Also: X96754 | 1.8081860000 |
| U96769 | Chondroadherin | 1.8027737000 |
| U83843 | HIV-1 Nef interacting protein (Nip7-1)/U83843 | 1.7996851000 |
| HG3495-HT3689 | Collagen Type Ix Alpha 1 | −1.8500333000 |
| L47276 | (cell line HL-60) alpha topoisomerase/L47276; Also: L47277 | −1.8492658000 |
| *M85220 | Heavy chain disease IgA chain CH3 region | 1.7895246000 |
| U26266 | Deoxyhypusine synthase/U26266; Also: U79262 | 1.7888751000 |
| U09366 | Zinc finger protein ZNF133 | −1.8413595000 |
| U17743 | JNK activating kinase (JNKK1); Also: L36870 | −1.8407332000 |
| Y08374 | GP-39 cartilage protein | −1.8399492000 |
| U65404 | Erythroid-specific TRANSCRIPTION FACTOR EKLF | 1.7849737000 |
| J02963 | Platelet glycoprotein IIb | −1.8363241000 |
| M84820 | Retinoid X receptor beta (RXR-beta); Also: X63522, X65463 | −1.8347385000 |
| Z35309 | Adenylyl cyclase | −1.8325089000 |
| D87457 | KIAA0281 | 1.7785130000 |
| X74328 | CB2 (peripheral) cannabinoid receptor | 1.7792356000 |
| X79483 | ERK6 extracellular signal regulated kinase | 1.7785130000 |
| Z16411 | Phospholipase c; Also: U26425, Z37544_rna1 | 1.7817554000 |
| Z80345 | SCAD; Also: M26393 | 1.7813963000 |
| D49817 | Fructose 6-phosphate 2-kinase/fructose 2 6-bisphosphatase | 1.7767012000 |
| X97444 | Transmembrane protein Tmp21-IIex/X97444 | 1.7726884000 |
| Y07566 | RIT protein | −1.8238002000 |
| M36089 | DNA-repair protein (XRCC1) | 1.7723217000 |
| X86018 | MUF1 protein | 1.7671559000 |
| Y12478 | CHD5 protein | 1.7635395000 |
| Z69881 | Adenosine triphosphatase calcium | −1.8223316000 |
| X60655 | EVX1 | 1.7558749000 |
| D14686 | Glycine cleavage system T-protein | −1.8098962000 |
| U01160 | Transmembrane 4 superfamily protein (SAS) | −1.8108715000 |
| HG162-HT3165 | Tyrosine Kinase Receptor Axl 2 | 1.7516639000 |
| K02054 | Gastrin-releasing peptide | −1.8056707000 |
| L00137 | Growth hormone releasing factor | −1.8029447000 |
| V00565 | Preproinsulin; Also: M10039 | 1.7431176000 |
| U08989 | Glutamate transporter | −1.8019180000 |
| U79280 | Clone 23575 | −1.8014895000 |
| M38258 | Retinoic acid receptor gamma 1 | 1.7359979000 |
| X69115 | ZNF37A zinc finger protein | −1.7964009000 |
| HG3033-HT3194 | Spliceosomal protein Sap 62 | 1.7284743000 |
| M23294 | Beta-hexosaminidase beta-subunit (HEXB) | 1.7275413000 |
| X14830 | Muscle acetylcholine receptor beta-subunit | −1.7894044000 |
| X98507 | Myosin-I beta | −1.7876376000 |
| D87078 | KIAA0235 | 1.7267272000 |
| U08049 | Peripheral myelin protein-22 (PMP22) non-coding 1A/U08049 | −1.7837250000 |
| U14910 | RPE-retinal G protein-coupled receptor (rgr) | −1.7830098000 |
| U90547 | Ro/SSA ribonucleoprotein homolog (RoRet) | −1.7831887000 |
| D28383 | ATP synthase B chain | 1.7185017000 |
| U70732 | Glutamate pyruvate transaminase (GPT) | 1.7178323000 |
| D10495 | Protein kinase C delta-type | 1.7168377000 |
| L78833 | Ifp35 from BRCA1, Rho7 and vatl | 1.7155856000 |
| M37457 | Na+, K+-ATPase catalytic subunit alpha-III isoform | 1.7147488000 |
| U77846 | Elastin | −1.7806773000 |
| U87972 | NAD+-isocitrate dehydrogenase/U87972 | −1.7804973000 |
| U35113 | Metastasis-associated mta1 | 1.7084209000 |
| X07876 | Irp prot (int-1 related protein) | −1.7772455000 |
| X90858 | Uridine phosphorylase | −1.7732377000 |
| Y08682 | Carnitine palmitoyltransferase I type I; Also: U62733, U62317 | −1.7767012000 |
| U20428 | SNC19 sequence | 1.7071442000 |
| L41919 | HIC-1 fragment | −1.7691926000 |
| M11186 | Prepro-oxytocin-neurophysin I (OXT) | −1.7693773000 |
| X62535 | Diacylglycerol kinase | −1.7712199000 |
| HG3523-HT4899 | Proto-Oncogene C-Myc; Also: L00058, HG3523-HT4900 | 1.6980926000 |
| X56687 | Autoantigen NOR-90; Also: X53461, X53390, U65487_rna1 | 1.6998235000 |
| J04449 | (clone NF 10) cytochrome P-450 nifedipine oxidase | −1.7654823000 |
| L40386 | DP-2; Also: U18422 | 1.6946052000 |
| L37199 | (clone cD24-1) Huntington's disease candidate regiont | 1.6879746000 |
| U52373 | Serine/threonine kinase MNB (mnb); Also: D85759, D86550 | 1.6916708000 |
| L18920 | MAGE-2 | −1.7604225000 |
| *M87789 | Hybridoma H210 anti-hepatitis A IgG V, C, CDR regions | 1.6826464000 |
| U40490 | Nicotinamide nucleotide transhydrogenase | 1.6852938000 |
| L43964 | (clone F-T03796) STM-2 | −1.7531999000 |
| X96753 | Chondroitin sulfate proteoglycan (MCSP) | −1.7562556000 |
| D42053 | KIAA0091 | 1.6794279000 |
| HG3437-HT3628 | Myelin Proteolipid protein; Also: M54927 | 1.6731273000 |
| M21665 | Beta-myosin heavy chain; Also: X52889 | 1.6762362000 |
| M31169 | Propionyl-CoA carboxylase beta-subunit/M31169 | 1.6720979000 |
| AF005361 | Importin alpha 6 | −1.7435098000 |
| M91196 | DNA-binding protein | −1.7448795000 |
| U35139 | NECDIN related protein | 1.6669857000 |
| M22324 | Aminopeptidase N | −1.7395723000 |
| *M74826 | Glutamate decarboxylase (GAD-2) | −1.7407573000 |
| X60957 | Tie putative receptor tyrosine kinase | −1.7393745000 |
| HG4518-HT4921 | Transcription Factor Btf3 Homolog M90355 | 1.6603911000 |

TABLE 6
Gene targets in MS spinal cord white matter from a sample with lymphocytic
inflammation, demyelination and axonal loss.

| Probe set | Gene description | log10 (ratio) fold change |
|---|---|---|
| *M63438 | Ig rearranged gamma chain V-J-C region; Also: X96754 | 2.8860030000 |
| AB000584 | TGF-beta superfamily protein | −2.9452516000 |
| L00389 | Cytochrome P-450 4 | −2.9300826000 |
| M84739 | Autoantigen calreticulin | 2.6676864000 |
| M13755 | Interferon-induced 17-kDa/15-kDa protein | −2.6319001000 |
| HG3945-HT4215 | Phospholipid Transfer protein | 2.4979657000 |
| *M87789 | Anti-hepatitis A IgG V, C, CDR regions; Also: J00221_2 | 2.4941391000 |
| D26561 | ORF E7 from papillomavirus 5b genome | −2.4064124000 |
| HG3033-HT3194 | Spliceosomal protein Sap 62 | 2.4704774000 |
| U22970 | 16-Jun (interferon-inducible peptide precursor) | −2.3853381000 |
| X93996 | AFX protein | 2.4455264000 |
| M21904 | 4F2 glycosylated heavy chain (4F2HC) antigen | 2.4151404000 |
| AB000895 | Cadherin FIB1 | −2.3181155000 |
| Y00757 | Polypeptide 7B2 | −2.3219607000 |
| M96684 | Pur (pur-alpha) | 2.3033041000 |
| D17716 | N-acetylglucosaminyltransferase V | −2.2915353000 |
| M16653 | Pancreatic elastase IIB | −2.2924222000 |
| HG3995-HT4265 | Cpg-Enriched Dna Clone S19 | −2.2683997000 |
| D10495 | Protein kinase C delta-type | 2.2642273000 |
| D63486 | KIAA0152 | 2.2668195000 |
| J04501 | Muscle glycogen synthase | 2.2589870000 |
| U37219 | Cyclophilin-like protein CyP-60 | −2.2589364000 |
| D43968 | AML1b protein | −2.2463139000 |
| X67325 | p27 | −2.2399248000 |
| U12707 | Wiskott-Aldrich syndrome protein (WASP); Also: U19927 | 2.2246625000 |
| *X64072 | CD18; Also: M15395 | 2.2272438000 |
| M33882 | p78 protein | −2.2166936000 |
| U45328 | Ubiquitin-conjugating enzyme (UBE2I); Also: U31882 | 2.1997661000 |
| U40279 | Beta-2 integrin alphaD subunit (ITGAD)/U40279 | −2.1967977000 |
| X53683 | LAG-1 | −2.1972116000 |
| *X13334 | CD14 myeloid cell-specific leucine-rich glycoprotein | 2.1776808000 |
| L41143 | Expressed pseudo TCTA at t(1; 3) translocation site | 2.1658892000 |
| U87964 | Putative G-protein (GP-1) | −2.1662080000 |
| M63573 | Secreted cyclophilin-like protein (SCYLP) | 2.1523334000 |
| HG3987-HT4257 | Cpg-Enriched Dna Clone E06 | −2.1621161000 |
| L00205 | K6b epidermal keratin type II | −2.1467480000 |
| X95406 | Cyclin E | −2.1339379000 |
| M77829 | Channel-like integral membrane protein (CHIP28) | 2.1191604000 |
| M61199 | Cleavage signal 1 protein | 2.1075491000 |
| U82310 | Unknown protein/U82310 | −2.1044871000 |
| M28825 | Thymocyte antigen CD1a | −2.0982975000 |
| U15655 | Ets domain protein ERF | 2.0989896000 |
| M23294 | Beta-hexosaminidase beta-subunit (HEXB) | 2.0948204000 |
| L32866 | Effector cell protease receptor-1 (EPR-1) | −2.0857364000 |
| K03189 | Chorionic gonadotropin beta subunit | 2.0920185000 |
| L76702 | B56-delta | 2.0888446000 |
| X13810 | OTF-2 lymphoid-specific transcription factor; Also: M36542 | −2.0805363000 |
| J02947 | Extracellular-superoxide dismutase (SOD3) | 2.0849336000 |
| D50402 | NRAMP1 | 2.0795430000 |
| X54637 | Tyk2 non-receptor protein tyrosine kinase | 2.0818871000 |
| AB000409 | MNK1 | −2.0695756000 |
| HG162-HT3165 | Tyrosine Kinase Receptor Axl 2 | 2.0771862000 |
| HG4243-HT4513 | Zinc Finger protein Znf155 | −2.0479560000 |
| **X05299 | (˜95%) major centromere autoantigen CENP-B | 2.0411952000 |
| HG2614-HT2710 | Collagen Type Viii Alpha 1 | 2.0360297000 |
| U02619 | TFIIIC Box B-binding subunit | 2.0366289000 |
| D87937 | Alpha(1,2)fucosyltransferase 5 | −2.0150452000 |
| M98045 | Folylpolyglutamate synthetase | −2.0156740000 |
| *M94250 | Retinoic acid inducible factor (MK) | 2.0295867000 |
| M63967 | Mitochondrial aldehyde dehydrogenase x | −2.0057166000 |
| Y11306 | Beta catenin/TCF-4 | 2.0193240000 |
| *U60269 | Putative ERVK envelope protein | −2.0017337000 |
| X59766 | Zn-alpha2-glycoprotein | −1.9977139000 |
| M38258 | Retinoic acid receptor gamma 1 | 2.0157788000 |
| D79998 | KIAA0176 | −1.9967305000 |
| U71364 | Serine protase inhibitor (P19) | −1.9973864000 |
| L02867 | 62 kDa paraneoplastic antigen | 2.0036759000 |
| L10386 | Transglutaminase E3 (TGASE3) | 1.9989129000 |
| M36429 | Transducin beta-2 subunit; Also: M16538 | 1.9967305000 |
| U80669 | Androgen regulated homeobox protein (NKX31) | −1.9772662000 |
| HG3928-HT4198 | SFTPA2D | 1.9860996000 |
| J04948 | Alkaline phosphatase (ALP-1) | 1.9865478000 |
| U17969 | Initiation factor eIF-5A | −1.9667282000 |
| M20681 | Glucose transporter-like protein-III (GLUT3) | −1.9596375000 |
| M24594 | Interferon-inducible 56 Kd protein | −1.9576073000 |
| M86826 | IGF binding protein complex acid-labile subunit a | 1.9715078000 |
| M21984 | (clone PWHTnT16) skeletal muscle Troponin T | −1.9555675000 |
| M35252 | CO-029 | −1.9542425000 |
| *U37408 | CtBP | 1.9580858000 |
| U32576 | Apolipoprotein apoC-IV (APOC4) | −1.9517017000 |
| M21389 | Keratin type II (58 kD) | −1.9454686000 |
| U07919 | Aldehyde dehydrogenase 6 | −1.9452223000 |
| L24774 | Delta3, delta2-CoA-isomerase; Also: Z25821_rna1, Z25820 | 1.9465301000 |
| U37519 | Aldehyde dehydrogenase (ALDH8) | −1.9301847000 |
| U12139 | Alpha1(XI) collagen (COL11A1)/U12139 | 1.9232440000 |
| U33920 | Clone lambda 5 semaphorin | 1.9253121000 |
| S75989 | Gamma-aminobutyric acid transporter type 3 | −1.9180303000 |
| V00565 | Preproinsulin; Also: M10039 | 1.9114240000 |
| U53442 | p38Beta MAP kinase | −1.9100905000 |
| M15465 | Pyruvate kinase type L; Also: D13243 | −1.9063350000 |
| K02100 | Ornithine transcarbamylase (OTC) | −1.8898617000 |
| X00734 | Beta-tubulin (5-beta) with ten Alu family members | 1.8830934000 |
| AB002315 | KIAA0317 | −1.8874766000 |
| X04470 | Antileukoprotease (ALP) from cervix uterus; Also: X04503 | 1.8810992000 |
| U15932 | Dual-specificity protein phosphatase | −1.8727388000 |
| X13766 | Beta-casein; Also: L10615, X17070 | −1.8754953000 |
| M58297 | Zinc finger protein 42 (MZF-1) | −1.8720105000 |
| D42053 | KIAA0091 | 1.8588379000 |
| S74445 | Cellular retinoic acid-binding protein | 1.8589298000 |
| U15131 | p126 (ST5) | 1.8576340000 |
| U28368 | Id-related helix-loop-helix protein Id4 | 1.8527849000 |
| AF006609 | RGS3 | 1.8510028000 |
| D49357 | S-adenosylmethionine synthetase | 1.8491122000 |
| U12595 | Tumor necrosis factor type 1 receptor associated protein TRAP1 | −1.8570308000 |
| U35234 | Protein tyrosine phosphatase sigma | 1.8379039000 |
| S78432 | Un-named-transcript-1 from SAS = transmembrane 4 protein | −1.8457180000 |
| L31881 | Nuclear factor I-X | 1.8350561000 |
| U93049 | SLP-76 associated protein | 1.8372727000 |
| U17743 | JNK activating kinase (JNKK1); Also: L36870 | −1.8407332000 |
| Z26256 | L-type calcium channel/Z26256 | 1.8286599000 |
| D25539 | KIAA0040 | −1.8345796000 |
| L07738 | DHP-sensitive calcium channel gamma subunit (CACNLG) | 1.8224950000 |
| X06956 | HALPHA44 alpha-tubulin | 1.8215490000 |
| Y10207 | CD171 protein/Y10207 | 1.8202015000 |
| X52056 | Spi-1 proto-oncogene | 1.8075350000 |
| S81294 | DCC = deleted in colorectal cancer/S81294 | −1.8149132000 |
| U28488 | Putative G protein-coupled receptor (AZ3B); Also: U62027 | 1.8041394000 |
| U22029 | Cytochrome P450 (CYP2A7) | −1.8080421000 |
| X92896 | ITBA2 protein | −1.8077041000 |
| L00137 | Growth hormone releasing factor | −1.8029447000 |
| X07315 | PP15 (placental protein 15) | 1.7856857000 |
| L13720 | Growth-arrest-specific protein (gas) | 1.7777892000 |
| J04469 | Mitochondrial creatine kinase (CKMT) | −1.7906370000 |
| U85767 | Myeloid progenitor inhibitory factor-1 MPIF-1 | −1.7895807000 |
| X14830 | Muscle acetylcholine receptor beta-subunit | −1.7894044000 |
| M74093 | Cyclin | −1.7830098000 |
| L40407 | Thyroid receptor interactor (TRIP9) | 1.7708520000 |
| X53002 | Integrin beta-5 subunit; Also: J05633 | 1.7701153000 |
| U87972 | NAD+-isocitrate dehydrogenase/U87972 | −1.7804973000 |
| M32053 | H19 RNA (spliced in silico) | 1.7634280000 |
| U27333 | Alpha (1,3) fucosyltransferase (FUT6), major transcript I | 1.7652959000 |
| L40393 | (clone S171) | 1.7570162000 |
| J04449 | (clone NF 10) cytochrome P-450 nifedipine oxidase | −1.7654823000 |
| U03494 | Transcription factor LSF | −1.7667845000 |
| U82306 | Unknown protein/U82306 | 1.7493498000 |
| X17094 | Furin | 1.7497363000 |
| M96980 | Myelin TRANSCRIPTION FACTOR 1 (MTF1) | 1.7425180000 |
| M20642 | Alkali myosin light chain 1; Also: X05451, M20643 | −1.7607993000 |
| S76942 | Dopamine D4 receptor; Also: L12398, HG944-HT944 | −1.7606109000 |
| U18018 | E1A enhancer binding protein (E1A-F) | −1.7604464000 |
| M81933 | Cdc25A | 1.7403627000 |
| HG620-HT620 | Tyrosine Phosphatase Epsilon | −1.7531999000 |
| Y10506 | CD110 protein/Y10506 | −1.7547305000 |
| U78524 | Gu binding protein | 1.7351995000 |
| M29540 | Carcinoembryonic antigen (CEA) | −1.7522406000 |
| U35113 | Metastasis-associated mta1 | 1.7283538000 |
| D50913 | KIAA0123 | −1.7435098000 |
| M22324 | Aminopeptidase N | −1.7395723000 |
| X55740 | Placental cDNA coding nucleotidase (EC 3135) | −1.7383841000 |
| S81737 | Alpha 1 syntrophin; Also: U40571 | 1.7185017000 |
| U03399 | T-complex protein 10A (TCP10A) | 1.7197455000 |
| M24351 | PTH-like hormone A | −1.7327287000 |
| Y09858 | Unknown protein | 1.7134905000 |
| U20428 | SNC19 sequence | −1.7109631000 |
| D13633 | KIAA0008 | −1.7281508000 |
| U55764 | Estrogen sulfotransferase | −1.7281508000 |
| D49817 | Fructose 6-phosphate 2-kinase/fructose 2 6-bisphosphatase | 1.7067178000 |
| U15197 | Histo-blood group ABO protein | 1.7058637000 |
| X89109 | Coronin; Also: D44497 | 1.7056006000 |
| HG896-HT896 | Thrombospondin 2 | −1.7248900000 |
| S70585 | Thyroid-stimulating hormone alpha subunit | −1.7226339000 |
| U25433 | Protein associated with tumorigenic conversion (CATR13) | −1.7252989000 |
| U82979 | Ig-like transcript-3 | −1.7236609000 |
| D28383 | ATP synthase B chain | 1.6985355000 |
| D29956 | KIAA0055 | 1.6994041000 |
| M57732 | Hepatic nuclear factor 1 (TCF1) | −1.7201593000 |
| U60521 | Protease proMch6 (Mch6) | −1.7220166000 |
| X52008 | Strychnine binding subunit of inhibitory glycine receptor | −1.7182940000 |
| AF015950 | Telomerase reverse transcriptase | 1.6932872000 |
| D78156 | RasGTPase activating protein | 1.6946052000 |
| M73077 | Glucocorticoid receptor repression factor 1 (GRF-1) | −1.7077830000 |
| U58681 | Neurogenic basic-helix-loop-helix protein (NeuroD2) | −1.7113854000 |
| HG273-HT273 | Lymphocyte Antigen Hla-G3; Also: J03027 | 1.6830470000 |
| S77361 | Transcript ch132/S77361 | 1.6826793000 |
| M81780 | SMPD1 | −1.7041505000 |
| U41804 | Putative T1/ST2 receptor binding protein precursor | −1.7056499000 |
| X04706 | Homeobox (clone HHO.c13); Also: X17360_rna1 | −1.7052221000 |
| U89896 | Casein kinase l gamma 2 | 1.6789734000 |
| HG4318-HT4588 | Lim-Domain Transcription Factor Lim-1; Also: U14755 | −1.6987528000 |
| U08021 | Nicotinamide N-methyltransferase (NNMT) | −1.7019995000 |
| X52228 | Secreted epithelial tumor mucin antigen | −1.7002709000 |
| X73079 | Encoding Polymeric Ig receptor | −1.6996932000 |
| X15331 | Phosphoribosylpyrophosphate synthetase subunit one | 1.6734817000 |
| S58733 | pp52 = B lymphocyte signal transduction | −1.6941663000 |
| D87077 | KIAA0240 | 1.6707096000 |
| U46461 | Dishevelled homolog (DVL) | 1.6688516000 |
| X86018 | MUF1 protein | 1.6720979000 |
| M81882 | Glutamate decarboxylase (GAD65) | −1.6917444000 |
| Z38133 | Myosin; Also: M36769 | −1.6899744000 |
| U83192 | Post-synaptic density protein 95 (PSD95) | 1.6637009000 |
| L43579 | (clone 110298)/L43579; Also: L43575 | 1.6584884000 |
| U21049 | DD96 | 1.6618127000 |
| L24203 | Ataxia-telangiectasia group D-associated protein | −1.6855178000 |
| U49248 | Canalicular multispecific organic anion transporter (cMOAT) | −1.6839471000 |
| M28219 | Low density lipoprotein receptor | 1.6532125000 |
| M64929 | Protein phosphatase 2A alpha subunit | 1.6536948000 |
| U08096 | Peripheral myelin protein-22 (PMP22) non-coding 1B/U08096 | 1.6565773000 |
| M27749 | Ig-related 14.1 protein | −1.6814674000 |
| S38742 | HOX11 = HOX11 homeodomain {homeobox}; Also: M75952 | −1.6780629000 |
| U45983 | G protein-coupled receptor GPR-CY6 | −1.6801088000 |
| X95240 | Cysteine-rich secretory protein-3; Also: X94323 | −1.6780629000 |
| J02854 | 20-kDa myosin light chain (MLC-2) | −1.6748611000 |
| D38081 | Thromboxane A2 receptor | 1.6459133000 |
| D82346 | HNSPC | 1.6449307000 |
| D88799 | Cadherin | 1.6468936000 |
| M59820 | Granulocyte colony-stimulating factor receptor (CSF3R) | 1.6444386000 |
| U33838 | NF-kappa-B p65delta3, spliced transcript | −1.6690843000 |
| AF002224 | E6-AP ubiquitin protein ligase 3A/promoter P1 | 1.6389882000 |
| X16260 | Inter-alpha-trypsin inhibitor subunit 3; Also: X69532_rna1 | 1.6379898000 |
| D21063 | KIAA0030 | −1.6641717000 |
| Z19574 | Cytokeratin 17 | −1.6662839000 |
| U68723 | Checkpoint suppressor 1 | 1.6359861000 |
| D90064 | CGM6 CD66b (NCA-95) | −1.6611025000 |
| U05012 | Receptor tyrosine kinase TrkC (NTRK3); Also: S76475 | −1.6618127000 |
| M14219 | Chondroitin/dermatan sulfate proteoglycan (PG40) core protein | 1.6324573000 |
| X97444 | Transmembrane protein Tmp21-llex/X97444 | 1.6294096000 |
| Z11502 | Intestine-specific annexin | 1.6304279000 |
| M84526 | Adipsin/complement factor D | 1.6235563000 |
| U81523 | Endometrial bleeding associated factor | 1.6268534000 |
| X87212 | Cathepsin C | 1.6242821000 |
| D29640 | KIAA0051; Also: L33075 | −1.6490914000 |
| U32674 | Orphan receptor GPR9 (GPR9); Also: X95876 | −1.6466487000 |
| X65873 | Kinesin (heavy chain) | 1.6154240000 |
| X70218 | Protein phosphatase X | 1.6133132000 |
| M59371 | Protein tyrosine kinase | −1.6414741000 |
| U43916 | Tumor-associated membrane protein homolog (TMP) | −1.6337209000 |
| U33429 | K+ channel beta 2 subunit | 1.6047659000 |
| X16706 | Fra-2 | 1.6036815000 |
| X90780 | Cardiac troponin I | 1.6063814000 |
| X76498 | Uterine bombesin receptor | −1.6276219000 |
| HG3893-HT4163 | Phosphoglucomutase 1 | 1.6009729000 |
| S83308 | SOX5 = Sry-related HMG box | 1.6009729000 |
| U64197 | Chemokine exodus | 1.5982432000 |
| K02777 | T-cell receptor active alpha-chain from Jurkat cell line | −1.6260836000 |
| U07794 | Tyrosine kinase (TXK) | −1.6227320000 |
| X98337 | Complement factor H-related protein 4; Also: X68679 | −1.6271097000 |
| D29992 | Placental protein 5 (PP5) | 1.5932861000 |
| X51698 | Spasmolytic polypeptide (SP); Also: U47292 | 1.5971465000 |
| AF005887 | ATF family member ATF6 | −1.6211763000 |
| HG3432-HT3618 | Fibroblast Growth Factor Receptor K-Sam | −1.6198756000 |
| HG537-HT537 | Collagen Type Viii Alpha 2 | −1.6188323000 |
| M60278 | Heparin-binding EGF-like growth factor | −1.6216955000 |
| M83738 | Protein-tyrosine phosphatase (PTPase MEG2) | −1.6175245000 |
| X99975 | hRTR/hGCNF protein | −1.6188323000 |
| U40223 | Uridine nucleotide receptor (UNR) | 1.5888317000 |
| M84820 | Retinoid X receptor beta (RXR-beta); Also: X63522, X65463 | −1.6172546000 |
| U04313 | Maspin | −1.6143698000 |
| U16997 | Orphan receptor ROR gamma | −1.6173935000 |

TABLE 7
Gene targets in MS spinal cord white matter from a sample with inflammation
by macrophages and demyelination.

| Probe set | Gene description | log10 (ratio) fold chang |
|---|---|---|
| *M63438 | Ig rearranged gamma chain, V-J-C region | 3.0608377000 |
| HG1614-HT1614 | Protein Phosphatase 1 Alpha Catalytic Subunit | −2.6832272000 |
| L76200 | Guanylate kinase (GUK1) | −2.6705781000 |
| U02619 | TFIIIC Box B-binding subunit | 2.7524326000 |
| M91083 | DNA-binding protein (HRC1) | 2.7402837000 |
| U35234 | Protein tyrosine phosphatase sigma | 2.7303381000 |
| M95787 | 22 kDa smooth muscle protein (SM22) | −2.6407546000 |
| M13755 | Interferon-induced 17-kDa/15-kDa protein | −2.6319001000 |
| U72507 | 40871 sequence | −2.5803547000 |
| *M87789 | Anti-hepatitis A IgG V, C, CDR regions | 2.5829467000 |
| L41143 | Pseudo TCTA at t(1; 3) translocation site | 2.5372864000 |
| S68616 | Na+/H+ exchanger NHE-1 isom | −2.5223464000 |
| X00734 | Beta-tubulin (5-beta) with ten Alu family members | 2.4960990000 |
| J04948 | Alkaline phosphatase (ALP-1) | 2.4679779000 |
| AB002318 | KIAA0320 | −2.4959950000 |
| M27826 | Endogenous retroviral protease | −2.4913967000 |
| X79882 | Irp | −2.4741434000 |
| S73885 | AP-4 = basic helix-loop-helix DNA-binding protein | −2.4527445000 |
| U12707 | Wiskott-Aldrich syndrome protein (WASP) | 2.4106085000 |
| X12517 | U1 small nuclear RNP-specific C protein | 2.4070508000 |
| D84361 | p52 and p64 N-Shc | −2.4451759000 |
| M21388 | Unproductively rearranged Ig mu-chain V-region VD | −2.4238600000 |
| D87452 | KIAA0263 | −2.4207394000 |
| U12139 | Alpha1(XI) collagen (COL11A1)/U12139 | 2.3716219000 |
| U45328 | Ubiquitin-conjugating enzyme (UBE2I) | 2.3701682000 |
| *U37408 | CtBP | 2.3659557000 |
| D26561 | ORF E7 from papillomavirus 5b genome | −2.4064124000 |
| U40490 | Nicotinamide nucleotide transhydrogenase | 2.3468418000 |
| M60299 | Alpha-1 collagen type II s 1 2 and 3/M60299 | 2.3402458000 |
| U22970 | 16-Jun (interferon-inducible peptide precursor) | −2.3853381000 |
| AF002224 | E6-AP ubiquitin protein ligase 3A/promoter P1 | 2.3358589000 |
| L77213 | Phosphomevalonate kinase | −2.3777159000 |
| U40223 | Uridine nucleotide receptor (UNR) | 2.3265407000 |
| HG162-HT3165 | Tyrosine Kinase Receptor Axl 2 | 2.3128118000 |
| J02947 | Extracellular-superoxide dismutase (SOD3) | 2.3059959000 |
| AF015910 | Unknown protein | −2.3407910000 |
| L08246 | Myeloid cell differentiation protein (MCL1) | −2.3375591000 |
| D87433 | KIAA0246 | −2.3191061000 |
| U86759 | Netrin-2 like protein (NTN2I); Also: U86758_rna1 | 2.2712606000 |
| X07315 | PP15 (placental prot 15) | 2.2678754000 |
| D86977 | KIAA0224 | −2.3159179000 |
| M22632 | Mitochondrial aspartate aminotransferase | −2.3137091000 |
| X71135 | Sox3 | 2.2587570000 |
| AB000895 | Cadherin FIB1 | −2.3095154000 |
| Z26256 | L-type calcium channel/Z26256 | 2.2564772000 |
| D42053 | KIAA0091 | 2.2499318000 |
| *D61391 | Phosphoribosypyrophosphate synthetase-associated protein 39 | −2.2951821000 |
| M58286 | TNF receptor; Also: M63121, M33294, X55313, M75866 | −2.2938596000 |
| U02566 | Receptor tyrosine kinase tif; Also: U18934 | −2.2947417000 |
| U60116 | Skeletal muscle LIM-prot SLIM2 | 2.2412974000 |
| X89416 | Protein phosphatase 5 | 2.2405866000 |
| D90084 | Pyruvate dehydrogenase alpha subunit | −2.2917018000 |
| M16653 | Pancreatic elastase IIB | −2.2924222000 |
| D25303 | Integrin alpha subunit | −2.2749656000 |
| X81420 | hHKb1 protein | 2.2188077000 |
| AD000092 | RAD23A homolog | −2.2698630000 |
| L19711 | Dystroglycan (DAG1) | −2.2611439000 |
| D31833 | Vasopressin V1b receptor; Also: L37112 | 2.2053397000 |
| J05582 | Pancreatic mucin; Also: J05581 | 2.2060018000 |
| L24774 | Delta3, delta2-CoA-isomerase; Also: Z25821_rna1, Z25820 | 2.2038512000 |
| M84739 | Autoantigen calreticulin | 2.2049335000 |
| U37219 | Cyclophilin-like protein CyP-60 | −2.2546151000 |
| L38517 | Indian hedgehog protein (IHH) | 2.1964718000 |
| U51010 | Nicotinamide N-methyltransferase 1 and 5 ing region | 2.1883659000 |
| M24400 | Chymotrypsinogen | 2.1859669000 |
| U05861 | Hepatic dihydrodiol dehydrogenase | −2.2392995000 |
| X67325 | p27 | −2.2399248000 |
| U23803 | Heterogeneous ribonucleoprotein A0 | 2.1817007000 |
| X54637 | Tyk2 non-receptor protein tyrosine kinase | 2.1769590000 |
| D15049 | Protein tyrosine phosphatase | −2.2322971000 |
| *X64072 | CD18; Also: M15395 | 2.1707017000 |
| AF000545 | Putative purinergic receptor P2Y10 | −2.2244036000 |
| D50855 | Ca-sensing receptor; Also: U20760, U20759 | −2.2251800000 |
| U37221 | Cyclophilin-like protein | 2.1582117000 |
| L02321 | Glutathione S-transferase (GSTM5) | −2.2208922000 |
| K03189 | Chorionic gonadotropin beta subunit | 2.1563977000 |
| M34344 | Platelet glycoprotein IIb (GPIIb) | −2.2134513000 |
| X04201 | Skeletal muscle tropomyosin | −2.2087772000 |
| *M85220 | Heavy chain disease IgA chain CH3 region | 2.1465908000 |
| X59842 | PBX2; Also: U89336_2, D28769_1, X80700_rna1 | 2.1397217000 |
| Z49254 | L23-related | −2.2020794000 |
| M21665 | Beta-myosin heavy chain; Also: X52889 | 2.1256439000 |
| M94167 | Heregulin-beta2 | 2.1230345000 |
| X52896 | Dermal fibroblast elastin; Also: HG2994-HT4851 | 2.1256439000 |
| D38305 | Tob | −2.1898507000 |
| U66559 | Anaplastic lymphoma kinase receptor | −2.1918002000 |
| D13635 | KIAA0010 | 2.1218880000 |
| M34376 | Beta-microseminoprotein (MSP); Also: X57928 | 2.1207384000 |
| X14767 | GABA-A receptor, beta 1 subunit | −2.1846914000 |
| X17651 | Myf-4 myogenic determination factor | −2.1869563000 |
| L38929 | Protein tyrosine phosphatase delta | 2.1154441000 |
| M25809 | Endomembrane proton pump subunit | 2.1134085000 |
| HG3570-HT3773 | Protein Phosphatase Inhibitor Homolog | −2.1780412000 |
| M92287 | Cyclin D3 (CCND3) | −2.1800542000 |
| M77698 | GLI-Krupple related protein (YY1) | −2.1748590000 |
| D43767 | Chemokine | −2.1684238000 |
| *U52518 | Grb2-related adaptor protein (Grap) | 2.0925452000 |
| U27333 | Alpha (1,3) fucosyltransferase (FUT6), major transcript I | 2.0845763000 |
| J05252 | Also: M95971 | 2.0731683000 |
| U23430 | Cholecystokinin type A receptor (CCK-A); Also: L19315 | 2.0766404000 |
| HG3934-HT4204 | G1 Phase-Specific | −2.1499116000 |
| X99142 | Hair keratin hHb6 | 2.0715138000 |
| L00205 | K6b epidermal keratin type II | −2.1467480000 |
| M35128 | Muscarinic acetylcholine receptor | −2.1447706000 |
| S85963 | hIRS-1 = rat insulin receptor substrate-1 homolog | 2.0629578000 |
| X63359 | UGT2BIO udp glucuronosyltransferase | 2.0668847000 |
| HG4518-HT4921 | Transcription Factor Btf3 Homolog M90355 | 2.0608866000 |
| M25269 | Tyrosine kinase (ELK1) onco | 2.0620176000 |
| HG2755-HT2862 | T-Plastin | −2.1371958000 |
| L25878 | p33/HEH epoxide hydrolase (EPHX) | −2.1326599000 |
| X95406 | Cyclin E | −2.1339379000 |
| D80003 | KIAA0181 | −2.1313780000 |
| M80563 | CAPL protein | −2.1310570000 |
| *U28811 | Cysteine-rich fibroblast growth factor receptor (CFR-1) | −2.1317790000 |
| HG4068-HT4338 | Phosphoprotein Tal2 | 2.0472749000 |
| S80335 | Integrin beta 7 subunit | 2.0451273000 |
| D14822 | CBFA2T1 | −2.1245042000 |
| J02783 | Thyroid hormone binding protein (p55) | −2.1270238000 |
| M36089 | DNA-repair protein (XRCC1) | 2.0374878000 |
| U42412 | AMP-activated protein kinase gamma-1 subunit | −2.1207384000 |
| X12433 | pHS1-2 with ORF homolog to membrane receptor proteins | −2.1131910000 |
| D88799 | Cadherin | 2.0236639000 |
| X15331 | Phosphoribosylpyrophosphate synthetase subunit one | 2.0203613000 |
| U65011 | Preferentially expressed antigen of melanoma (PRAME) | −2.1092410000 |
| D30755 | KIAA0113 | −2.1044871000 |
| D50930 | KIAA0140 | −2.1026052000 |
| U20428 | SNC19 sequence | 2.0117818000 |
| U83192 | Post-synaptic density protein 95 (PSD95) | 2.0096633000 |
| HG2825-HT2949 | Ret Transforming | −2.0982045000 |
| M28825 | Thymocyte antigen CD1a | −2.0982975000 |
| AC002086 | PAC clone DJ525N14/Xq23 | 2.0030295000 |
| L02867 | 62 kDa paraneoplastic antigen | 2.0034605000 |
| ***D16480 | Mitochondrial enoylCoA hydratase | 1.9986952000 |
| U65404 | Erythroid-specific TRANSCRIPTION FACTOR EKLF | 2.0002171000 |
| L10386 | Transglutaminase E3 (TGASE3) | 1.9951963000 |
| L05500 | Fetal brain adenylyl cyclase | 1.9921115000 |
| U15655 | Ets domain prot ERF | 1.9914476000 |
| M16405 | m4 muscarinic acetylcholine receptor | −2.0841292000 |
| X86570 | Acidic hair keratin 1 | −2.0854690000 |
| D82345 | NB thymosin beta | 1.9813655000 |
| U15131 | p126 (ST5) | 1.9820450000 |
| U17838 | Zinc finger protein RIZ | −2.0785474000 |
| M75106 | Prepro-plasma carboxypeptidase B | 1.9738203000 |
| D12625 | Neurofibromin | 1.9633155000 |
| U83843 | HIV-1 Nef interacting prot (Nip7-1)/U83843 | 1.9644953000 |
| U96769 | Chondroadherin | 1.9654369000 |
| Z48512 | XG (clone PEP6)/Z48512 | −2.0719740000 |
| D87078 | KIAA0235 | 1.9580858000 |
| M36429 | Transducin beta-2 subunit; Also: M16538 | 1.9609462000 |
| M58026 | NB-1 | 1.9609462000 |
| U33920 | Clone lambda 5 semaphorin | 1.9607086000 |
| D83920 | Uterus ficolin-1 | 1.9572406000 |
| X80910 | PPP1CB | −2.0673499000 |
| AB000897 | Cadherin FIB3 | −2.0600365000 |
| M86933 | Amelogenin (AMELY); Also: M55419, U79549_rna1, M55418 | −2.0580462000 |
| X79066 | ERF-1 | −2.0611559000 |
| X60486 | H4/g H4 histone | −2.0529824000 |
| D28532 | Renal Na+-dependent phosphate cotransporter | −2.0486360000 |
| L41668 | UDP Galactose 4 epimerase | −2.0491211000 |
| U16031 | TRANSCRIPTION FACTOR IL-4 Stat | −2.0447357000 |
| U84540 | Dystrobrevin isom DTN-3 (DTN)/U84540 | −2.0471775000 |
| X01715 | Acetylcholine receptor gamma subunit precursor | −2.0451273000 |
| HG3033-HT3194 | Spliceosomal protein Sap 62 | 1.9207327000 |
| L41066 | NF-AT3 | 1.9175055000 |
| U57057 | WD protein IR10 | 1.9214263000 |
| AF001294 | IPL | −2.0404143000 |
| M60614 | Wilms tumor (WIT-1) associated protein | −2.0378248000 |
| HG2417-HT2513 | Dynein Heavy Chain | 1.9066044000 |
| M61199 | Cleavage signal 1 protein | 1.9057959000 |
| J04611 | Lupus p70 (Ku) autoantigen protein | −2.0357298000 |
| L38503 | Glutathione S-transferase theta 2 (GSTT2) | −2.0343276000 |
| X17098 | PSG10 pregnancy specific glycoprotein 10 | 1.9014583000 |
| X74328 | CB2 (peripheral) cannabinoid receptor | 1.8987252000 |
| D82060 | Kidney histidine rich putative membrane protein | −2.0292823000 |
| J04056 | Carbonyl reductase | −2.0295867000 |
| M23263 | Androgen receptor | −2.0285713000 |
| HG3945-HT4215 | Phospholipid Transfer protein | 1.8887410000 |
| M23294 | Beta-hexosaminidase beta-subunit (HEXB) | 1.8878985000 |
| M96684 | Pur (pur-alpha) | 1.8895818000 |
| L17327 | Pre-T/NK cell associated protein (3B3) | −2.0245883000 |
| M19888 | Small proline rich protein (sprl), clone 128 | −2.0258177000 |
| S79873 | H-lamp-2 = lysosome-associated membrane protein-2 | −2.0271457000 |
| X57351 | 1-8D from interferon-inducible family | −2.0254083000 |
| X74819 | Cardiac troponin T | 1.8825245000 |
| M55621 | N-acetylglucosaminyltransferase I (GlcNAc-TI) | 1.8793826000 |
| Z36714 | Cyclin F | 1.8788089000 |
| D84239 | lgG Fc binding protein | −2.0175732000 |
| S72370 | Pyruvate carboxylase | −2.0198430000 |
| U24576 | Breast tumor autoantigen sequence | −2.0212927000 |
| HG172-HT3924 | Spermidine/Spermine N1-Acetyltransferase | 1.8767950000 |
| X63755 | High-sulphur keratin | 1.8709888000 |
| D87937 | Alpha(1,2)fucosyltransferase 5 | −2.0150452000 |
| M62324 | Modulator recognition factor I (MRF-1) | −2.0143105000 |
| U22377 | Zn-15 related zinc finger protein (rlf) | 1.8588379000 |
| D28588 | KIAA0048 | −2.0045363000 |
| M63967 | Mitochondrial aldehyde dehydrogenase x | −2.0057166000 |
| X02176 | Complement component C9; Also: K02766 | 1.8488047000 |
| D86983 | KIAA0230 | 1.8450337000 |
| D49818 | Fructose 6-phosphate 2-kinase/fructose 2 6-bisphosphatase | 1.8366405000 |
| L29218 | Clk2 | 1.8374843000 |
| U09002 | NMDA receptor modulatory subunit 2A (hNR2A) | 1.8334659000 |
| HG4683-HT5108 | Tumor Necrosis Factor Receptor 2 Associated protein Trap3 | −1.9940971000 |
| L13391 | Helix-loop-helix basic phosphoprotein (G0S8) | −1.9964022000 |
| M98447 | Keratinocyte transglutaminase | −1.9956352000 |
| U71364 | Serine protase inhibitor (P19) | −1.9973864000 |
| X63629 | P cadherin | −1.9925535000 |
| X80695 | OXA1Hs | −1.9965117000 |
| X98482 | TNNT2 11/X98482 | 1.8299467000 |
| X59618 | RR2 small subunit ribonucleotide reductase | 1.8267225000 |
| X93996 | AFX protein | 1.8234742000 |
| D29992 | Placental protein 5 (PP5) | 1.8175654000 |
| J04970 | Carboxypeptidase M | 1.8224950000 |
| X04729 | Plasminogen activator inhibitor type 1 N-terminus/X04729 | 1.8175654000 |
| D31797 | CD40 ligand | −1.9855387000 |
| HG3578-HT3781 | Autoimmune Antigen Thyroid Disease-Related Antigen | 1.8123589000 |
| *M13241 | N-myc | 1.8081145000 |
| U05875 | Interferon gamma receptor accessory factor-1 (AF-1) | 1.8044061000 |
| X04500 | Prointerleukin 1 beta | 1.8068580000 |
| X92106 | Bleomycin hydrolase | 1.8020893000 |
| U21049 | DD96 | 1.7944880000 |
| U91931 | AP-3 complex beta3A subunit | 1.7944880000 |
| HG4593-HT4998 | Sodium Channel 1 | −1.9746268000 |
| M15881 | Uromodulin (Tamm-Horsfall glycoprotein) | −1.9748570000 |
| D80004 | KIAA0182 | −1.9684829000 |
| M13699 | Ceruloplasmin (ferroxidase) | 1.7788745000 |
| D87449 | KIAA0260 | 1.7770642000 |
| M36803 | Hemopexin | 1.7759743000 |
| S62028 | Recoverin; Also: S43855 | 1.7756104000 |
| X06182 | C-kit proto-oncogene; Also: HG2549-HT3951 | 1.7745170000 |
| M55682 | Cartilage matrix protein (CMP); Also: M55683 | 1.7715875000 |
| U17077 | BENE | 1.7678976000 |
| HG3921-HT4191 | Homeotic protein C6, Class I | −1.9577270000 |
| M20681 | Glucose transporter-like protein-III (GLUT3) | −1.9596375000 |
| U78180 | Sodium channel 2 (hBNaC2) | −1.9604708000 |
| D88795 | Cadherin | −1.9559282000 |
| L34587 | RNA polymerase II elongation factor SIII p15 subunit | −1.9548453000 |
| M21984 | (clone PWHTnT16) skeletal muscle Troponin T | −1.9555675000 |
| U37248 | Alpha-mannosidase (6A8) | −1.9553269000 |
| U45285 | Specific 116-kDa vacuolar proton pump subunit (OC-116 KDa) | −1.9530345000 |
| Z29083 | 5T4 Oncofetal antigen | −1.9550862000 |
| X99140 | Hair keratin hHb5 | 1.7489629000 |

TABLE 8
Gene targets in MS spinal cord white matter from a sample with inflammation
by macrophages and lymphocytes and demyelination.

| Probe set | Gene description | log10 (ratio) fold change |
|---|---|---|
| *M63438 | Ig rearranged gamma chain, V-J-C region; Also: X96754 | 3.5479855000 |
| L76200 | Guanylate kinase (GUK1) | −2.6727442000 |
| *M87789 | Anti-hepatitis A IgG V, C, CDR regions; Also: J00221_2 | 2.9066259000 |
| X74570 | Gal-beta(1-3/1-4)GlcNAc alpha-23-sialyltransferase | −2.5341531000 |
| HG3033-HT3194 | Spliceosomal protein Sap 62 | 2.6505720000 |
| HG3945-HT4215 | Phospholipid Transfer protein | 2.6058005000 |
| D84361 | p52 and p64 N-Shc | −2.4451759000 |
| D50402 | NRAMP1 | 2.4524994000 |
| D26561 | ORF E7 from papillomavirus 5b genome | −2.4064124000 |
| U22970 | 16-Jun (interferon-inducible peptide precursor); Also: U22970 | −2.3853381000 |
| L27080 | Melanocortin 5 receptor (MC5R) | 2.3691787000 |
| M84739 | Autoantigen calreticulin | 2.3240457000 |
| D49817 | Fructose 6-phosphate 2-kinase/fructose 2 6-bisphosphatase | 2.3082442000 |
| M96326 | Azurocidin | −2.3481101000 |
| HG3286-HT3463 | Crystallin Alpha A | −2.3455208000 |
| J03600 | Lipoxygenase | −2.3282267000 |
| L32976 | Protein kinase (MLK-3) | −2.3323880000 |
| M80397 | DNA polymerase delta catalytic subunit; Also: M81735 | −2.3273589000 |
| J04948 | Alkaline phosphatase (ALP-1) | 2.2624986000 |
| AB000895 | Cadherin FIB1 | −2.3181155000 |
| Y00757 | Polypeptide 7B2 | −2.3219607000 |
| X99142 | Hair keratin hHb6 | 2.2411728000 |
| *D61391 | Phosphoribosypyrophosphate synthase-associated protein39 | −2.2951821000 |
| Y08409 | Spot14 | −2.2876898000 |
| L14565 | Peripherin (PRPH) s 1-9 | 2.2002210000 |
| D13988 | Rab GDI | 2.1970461000 |
| M23294 | Beta-hexosaminidase beta-subunit (HEXB) | 2.1922189000 |
| U15131 | p126 (ST5) | 2.1918421000 |
| HG2614-HT2710 | Collagen Type Viii Alpha 1 | 2.1829707000 |
| U37219 | Cyclophilin-like protein CyP-60 | −2.2589364000 |
| U56816 | Kinase Myt1 (Myt1) | −2.2537014000 |
| U45328 | Ubiquitin-conjugating enzyme (UBE2I); Also: U31882 | 2.1722622000 |
| X67325 | p27 | −2.2399248000 |
| *M94250 | Retinoic acid inducible factor (MK) | 2.1555486000 |
| M55621 | N-acetylglucosaminyltransferase I (GlcNAc-TI) | 2.1510939000 |
| J02947 | Extracellular-superoxide dismutase (SOD3); Also: U10116 | 2.1296738000 |
| U35234 | Protein tyrosine phosphatase sigma | 2.1262938000 |
| X53683 | LAG-1 | −2.1972116000 |
| X17651 | Myf-4 myogenic determination factor | −2.1869563000 |
| HG3570-HT3773 | Protein Phosphatase Inhibitor Homolog | −2.1780412000 |
| D10495 | Protein kinase C delta-type | 2.0939642000 |
| M38258 | Retinoic acid receptor gamma 1 | 2.0938243000 |
| M27161 | MHC class I CD8 alpha-chain (Leu-2/T8) | −2.1755118000 |
| U13706 | ELAV-like neuronal protein 1 isom Hel-N2 (Hel-N1)/U13706 | −2.1692335000 |
| X15331 | Phosphoribosylpyrophosphate synthetase subunit one | 2.0852549000 |
| U87964 | Putative G-protein (GP-1) | −2.1662080000 |
| U77643 | K12 protein precursor | 2.0813599000 |
| HG3987-HT4257 | Cpg-Enriched Dna Clone E06 | −2.1621161000 |
| U12707 | Wiskott-Aldrich syndrome protein (WASP); Also: U19927 | 2.0746519000 |
| AB000462 | SH3 binding RES4-23A | −2.1517587000 |
| U12139 | Alpha1(XI) collagen (COL11A1)/U12139 | 2.0644580000 |
| X54637 | Tyk2 non-receptor protein tyrosine kinase | 2.0618105000 |
| D49490 | Disulfide isomerase-related protein | −2.1380657000 |
| X95406 | Cyclin E | −2.1339379000 |
| M63582 | Preprothyrotropin-releasing hormone | −2.1266184000 |
| D14827 | Tax helper protein 1 | −2.1194209000 |
| L29433 | Factor X (blood coagulation factor) | 2.0325785000 |
| U93049 | SLP-76 associated protein | 2.0340667000 |
| D83784 | KIAA0198 | −2.1156937000 |
| D63486 | KIAA0152 | 2.0280830000 |
| L16991 | Thymidylate kinase (CDC8) | 2.0283882000 |
| U15197 | Histo-blood group ABO protein | 2.0293026000 |
| HG3242-HT4231 | Calcium Channel, Voltage-Gated, Alpha 1e Subunit, 3 | −2.1080574000 |
| U65011 | Preferentially expressed antigen of melanoma (PRAME) | −2.1092410000 |
| U82310 | Unknown protein/U82310 | −2.1044871000 |
| HG2290-HT2386 | Calcitonin | −2.1007151000 |
| M28825 | Thymocyte antigen CD1a | −2.0982975000 |
| X57110 | HG642-HT642 and others | −2.0995943000 |
| M31776 | M25296 and others | 2.0134061000 |
| Y08263 | AAD14 protein | −2.0928083000 |
| U77968 | Neuronal PAS1 (NPAS1) | 2.0047512000 |
| L38487 | Estrogen receptor-related protein (hERRa1) | −2.0839503000 |
| M16405 | m4 muscarinic acetylcholine receptor | −2.0841292000 |
| U96769 | Chondroadherin | 1.9994350000 |
| U83192 | Post-synaptic density protein 95 (PSD95) | 1.9963584000 |
| X79066 | ERF-1 | −2.0739931000 |
| AB000409 | MNK1 | −2.0695756000 |
| Z48512 | XG (clone PEP6)/Z48512 | −2.0719740000 |
| X60486 | H4/g H4 histone | −2.0529824000 |
| L24774 | Delta3, delta2-CoA-isomerase; Also: Z25821_rna1, Z25820 | 1.9747715000 |
| U79273 | Clone 23933 sequence | 1.9728737000 |
| X04898 | Apolipoprotein AII | 1.9733099000 |
| X07315 | PP15 (placental protein 15) | 1.9728968000 |
| L41668 | UDP Galactose 4 epimerase | −2.0491211000 |
| AF008445 | Phospholipid scramblase | 1.9705328000 |
| AF001294 | IPL | −2.0447357000 |
| D13636 | KIAA0011 | −2.0416887000 |
| L08488 | Inositol polyphosphate 1-phosphatase | 1.9596625000 |
| Z48314 | Apomucin; Also: U06711 | 1.9617769000 |
| J04056 | Carbonyl reductase | −2.0295867000 |
| M21904 | 4F2 glycosylated heavy chain (4F2HC) antigen | 1.9515075000 |
| *X64072 | CD18; Also: M15395 | 1.9479481000 |
| U23430 | Cholecystokinin type A receptor (CCK-A); Also: L19315 | 1.9457393000 |
| M62324 | Modulator recognition factor I (MRF-1) | −2.0143105000 |
| D21851 | KIAA0028 | −2.0075344000 |
| X64037 | RNA polymerase II associated protein RAP74 | −2.0115704000 |
| U83303 | GCP-2 (granulocyte chemotactic protein-2) | −2.0065730000 |
| HG162-HT3165 | Tyrosine Kinase Receptor Axl 2 | 1.9327020000 |
| M81933 | Cdc25A | 1.9346247000 |
| X04500 | Prointerleukin 1 beta | 1.9345489000 |
| M61199 | Cleavage signal 1 protein | 1.9318391000 |
| U28831 | Protein immuno-reactive with anti-PTH polyclonal antibodies | 1.9287542000 |
| U35113 | Metastasis-associated mta1 | 1.9319915000 |
| X92106 | Bleomycin hydrolase | 1.9279859000 |
| D79998 | KIAA0176 | −1.9967305000 |
| HG4683-HT5108 | Tumor Necrosis Factor Receptor 2 Associated protein Trap3 | −1.9940971000 |
| U71364 | Serine protase inhibitor (P19) | −1.9973864000 |
| U78524 | Gu binding protein | 1.9260336000 |
| X17098 | PSG10 pregnancy specific glycoprotein 10 | 1.9229848000 |
| M15881 | Uromodulin (Tamm-Horsfall glycoprotein) | −1.9748570000 |
| D42053 | KIAA0091 | 1.9100104000 |
| M19311 | Calmodulin | 1.9089673000 |
| HG3921-HT4191 | Homeotic protein C6, Class I | −1.9577270000 |
| U78180 | Sodium channel 2 (hBNaC2) | −1.9604708000 |
| L41143 | Expressed pseudo TCTA at t(1; 3) translocation site | 1.8975177000 |
| M21984 | (clone PWHTnT16) skeletal muscle Troponin T | −1.9555675000 |
| Z29083 | 5T4 Oncofetal antigen | −1.9550862000 |
| L36463 | Ras inhibitor (Rin1) | 1.8962506000 |
| U32576 | Apolipoprotein apoC-IV (APOC4) | −1.9517017000 |
| X13839 | Vascular smooth muscle alpha-actin | −1.9479070000 |
| L35240 | Enigma | −1.9442358000 |
| X07695 | Cytokeratin 4 | −1.9460836000 |
| X89101 | Fas (Apo-1, CD95)./X89101; Also: X83493, X63717, X83492 | −1.9459607000 |
| U15655 | Ets domain protein ERF | 1.8889934000 |
| U02082 | Guanine nucleotide regulatory protein (tim1) | −1.9407654000 |
| U11690 | Faciogenital dysplasia (FGD1) | 1.8867726000 |
| U65404 | Erythroid-specific TRANSCRIPTION FACTORS EKLF | 1.8825245000 |
| U82818 | UCP3S/U82818 | 1.8860826000 |
| M96980 | Myelin TRANSCRIPTION FACTORS 1 (MTF1) | 1.8786190000 |
| X93996 | AFX protein | 1.8819835000 |
| D10511 | Mitochondrial acetoacetyl-CoA thiolase | −1.9274987000 |
| D79985 | KIAA0163 | −1.9194703000 |
| M84424 | Cathepsin E (CTSE) | −1.9196010000 |
| M36089 | DNA-repair protein (XRCC1) | 1.8706560000 |
| U02619 | TFIIIC Box B-binding subunit | 1.8707549000 |
| U17566 | 65 kDa hydrophobic protein | 1.8723894000 |
| *L05624 | MAP kinase kinase; Also: L11284 | 1.8667598000 |
| U28488 | Putative G protein-coupled receptor (AZ3B); Also: U62027 | 1.8656961000 |
| J03171 | Interferon-alpha receptor (HuIFN-alpha-Rec) | −1.9122221000 |
| U53442 | p38Beta MAP kinase | −1.9100905000 |
| M15465 | Pyruvate kinase type L; Also: D13243 | −1.9063350000 |
| X87870 | Hepatocyte nuclear factor 4a | −1.9037680000 |
| HG4390-HT4660 | Ribosomal protein L18a Homolog | 1.8601583000 |
| *U37408 | CtBP | 1.8621016000 |
| Z78289 | (clone 1D2)/Z78289 | 1.8617404000 |
| M11722 | Terminal transferase | 1.8534244000 |
| HG831-HT831 | Potassium Channel | −1.8930679000 |
| J00220 | IGHA1 from Ig germline H-chain G-E-A region A: gamma-3 5 | 1.8475357000 |
| M59820 | Granulocyte colony-stimulating factor receptor (CSF3R) | 1.8484047000 |
| K02100 | Ornithine transcarbamylase (OTC) | −1.8898617000 |
| L21998 | Intestinal mucin (MUC2) | −1.8902813000 |
| U06698 | Neuronal kinesin heavy chain | −1.8904210000 |
| U68233 | Farnesol receptor HRR-1 (HRR-1) | −1.8884603000 |
| M34516 | Omega light chain protein 14.1 (Ig lambda chain related) | 1.8469395000 |
| M36429 | Transducin beta-2 subunit; Also: M16538 | 1.8443529000 |
| U29615 | Chitotriosidase precursor | 1.8448808000 |
| U60116 | Skeletal muscle LIM-protein SLIM2 | 1.8436687000 |
| X60655 | EVX1 | 1.8447256000 |
| M74525 | HHR6B (yeast RAD 6 homolog) | −1.8828090000 |
| Z71389 | Skin-antimicrobial-peptide 1 (SAP1)/Z71389 | −1.8800987000 |
| U15932 | Dual-specificity protein phosphatase | −1.8727388000 |
| X13766 | Beta-casein; Also: L10615, X17070 | −1.8754953000 |
| D82346 | HNSPC | 1.8354052000 |
| AF009674 | Axin | −1.8701112000 |
| U65093 | Msg1-related 1 (mrg1) | −1.8704039000 |
| U67733 | Cyclic nucleotide phosphodiesterase PDE2A3 | 1.8279505000 |
| *X13334 | CD14 myeloid cell-specific leucine-rich glycoprotein | 1.8282409000 |
| AF000231 | Rab11a GTPase | −1.8662873000 |
| D83782 | KIAA0199 | −1.8634716000 |
| Z29331 | (23 k/3) ubiquitin-conjugating enzyme UbcH2 | 1.8257506000 |
| Y10262 | EYA3/Y10262; Also: U81602 | −1.8571817000 |
| L47276 | (cell line HL-60) alpha topoisomerase/L47276; Also: L47277 | −1.8492658000 |
| M57567 | ADP-ribosylation factor (hARF5) | −1.8489585000 |
| U51587 | Golgi complex autoantigen golgin-97 | −1.8501866000 |
| U59058 | Beta-A3/A1 crystallin (CYRBA3/A1); Also: M14306 | −1.8490726000 |
| HG909-HT909 | Mg81 | 1.8123176000 |
| U17743 | JNK activating kinase (JNKK1); Also: L36870 | −1.8407332000 |
| J05582 | Pancreatic mucin; Also: J05581 | 1.8041208000 |
| Z35309 | Adenylyl cyclase | −1.8325089000 |
| L13720 | Growth-arrest-specific protein (gas) | 1.8010262000 |
| U83843 | HIV-1 Nef interacting protein (Nip7-1)/U83843 | 1.7976829000 |
| X70683 | SOX-4 protein | 1.7998573000 |
| M60746 | Histone H3.1 (H1F3) | −1.8265607000 |
| U27333 | Alpha (1,3) fucosyltransferase (FUT6), major transcript I | 1.7928468000 |
| U33920 | Clone lambda 5 semaphorin | 1.7951150000 |
| Z69881 | Adenosine triphosphatase calcium | −1.8223316000 |
| U08096 | Peripheral myelin protein-22 (PMP22) 1B/U08096 | 1.7898979000 |
| U48861 | Beta 4 nicotinic acetylcholine receptor subunit | 1.7900387000 |
| S81294 | DCC = deleted in colorectal cancer/S81294 | −1.8149132000 |
| L01406 | Growth hormone-releasing hormone receptor | 1.7840464000 |
| M91585 | Br140 | 1.7844746000 |
| D11094 | MSS1 | 1.7794161000 |
| L31881 | Nuclear factor I-X | 1.7732377000 |
| M24486 | Prolyl 4-hydroxylase alpha subunit; Also: M24487, U14620_1 | 1.7737864000 |
| HG4115-HT4385 | Olfactory Receptor Or17-210 | −1.7984780000 |
| L38929 | Protein tyrosine phosphatase delta | 1.7706311000 |
| U43843 | H-neuro-d4 prot | −1.7955324000 |
| M13981 | Inhibin A-subunit | 1.7638022000 |
| M25269 | Tyrosine kinase (ELK1) | 1.7632033000 |
| U67611 | Mouse transaldolase/U67611 | 1.7644277000 |
| Z26256 | L-type calcium channel/Z26256 | 1.7674898000 |
| X14830 | Muscle acetylcholine receptor beta-subunit | −1.7894044000 |
| L22548 | Collagen type XVIII alpha 1 (COL18A1) | 1.7594412000 |
| X53296 | IRAP; Also: X64532_rna1, X52015 | 1.7616648000 |
| X81420 | hHKb1 prot | 1.7594729000 |
| D49818 | Fructose 6-phosphate 2-kinase/fructose 2 6-bisphosphatase | 1.7560272000 |
| HG3884-HT4154 | Homeotic protein Hpx-42 | 1.7532382000 |
| M64929 | Protein phosphatase 2A alpha subunit | 1.7537745000 |
| U06088 | N-acetylgalactosamine 6-sulphatase (GALNS) | −1.7779703000 |
| U77846 | Elastin | −1.7806773000 |
| U87972 | NAD+-isocitrate dehydrogenase/U87972 | −1.7804973000 |
| D49357 | S-adenosylmethionine synthetase | 1.7486144000 |
| X86401 | L-arginine: glycine amidinotransferase; Also: S68805 | 1.7521582000 |
| X07876 | Irp protein (int-1 related protein) | −1.7772455000 |
| X52011 | MYF6 encoding a muscle determination factor | −1.7734207000 |
| *M24766 | Alpha-2 collagen type IV (COL4A2); Also: X05610 | 1.7465174000 |
| M58026 | NB-1 | 1.7429214000 |
| U22377 | Zn-15 related zinc finger protein (rlf) | 1.7474118000 |
| U28368 | Id-related helix-loop-helix protein Id4 | 1.7455432000 |
| X13293 | B-myb | 1.7445668000 |
| X53002 | Integrin beta-5 subunit; Also: J05633 | 1.7436665000 |
| L41919 | HIC-1 fragment | −1.7691926000 |
| M11186 | Prepro-oxytocin-neurophysin I (OXT) | −1.7693773000 |
| M96684 | Pur (pur-alpha) | 1.7398096000 |
| X65873 | Kinesin (heavy chain) | 1.7411122000 |
| J04449 | (clone NF 10) cytochrome P-450 nifedipine oxidase | −1.7654823000 |
| U28131 | HMGI-C chimeric transcript | −1.7626786000 |
| X87160 | Epithelial amiloride-sensitive sodium channel gamma | 1.7337989000 |
| M20642 | Alkali myosin light chain 1; Also: X05451, M20643 | −1.7607993000 |
| U59111 | Dermatan sulfate proteoglycan 3 (DSPG3) | −1.7585334000 |
| M77829 | Channel-like integral membrane protein (CHIP28) | 1.7323536000 |
| X16260 | Inter-alpha-trypsin inhibitor subunit 3; Also: X69532_rna1 | 1.7297721000 |
| X68090 | Fc-gamma-RIIA IgG Fc receptor class IIA/X68090 | 1.7286378000 |
| X68836 | S-adenosylmethionine synthetase | 1.7323133000 |
| HG620-HT620 | Tyrosine Phosphatase Epsilon | −1.7531999000 |
| L43964 | (clone F-T03796) STM-2 | −1.7531999000 |
| X96753 | Chondroitin sulfate proteoglycan (MCSP) | −1.7562556000 |
| Y10506 | CD110 protein/Y10506 | −1.7547305000 |
| M91083 | DNA-binding protein (HRC1) | 1.7237839000 |
| L34155 | Laminin-related protein (LamA3) | −1.7485756000 |
| X66362 | PCTAIRE-3 serine/threonine protein kinase | −1.7478001000 |
| AF005361 | Importin alpha 6 | −1.7435098000 |
| D50913 | KIAA0123 | −1.7435098000 |
| M91196 | DNA-binding protein (HRC1) | −1.7448795000 |
| X17094 | Furin | 1.7140782000 |
| X56199 | XIST a (locus DXS399E) | 1.7144136000 |
| X90872 | gp25L2 protein | 1.7174239000 |
| M22324 | Aminopeptidase N | −1.7395723000 |
| *M74826 | Glutamate decarboxylase (GAD-2) | −1.7407573000 |
| U71203 | Rit; Also: Y07566 | −1.7377888000 |
| X63359 | UGT2BIO udp glucuronosyltransferase | 1.7031624000 |
| X97444 | Transmembrane protein Tmp21-liex./X97444 | 1.7065898000 |

TABLE 9
Gene targets in MS spinal cord white matter from a sample with axonal loss.

| Probe set | Gene description | log10 (ratio) fold change |
|---|---|---|
| *M63438 | Ig rearranged gamma chain, V-J-C region; Also: X96754 | 3.9579713000 |
| AB000584 | TGF-beta superfamily protein | −2.9495730000 |
| *M87789 | Anti-hepatitis A IgG V, C, CDR regions; J00221 | 3.3816210000 |
| L76200 | Guanylate kinase (GUK1) | −2.6727442000 |
| M27504 | Topoisomerase type II (Topo II)/M27504/Also: Z15115 | 3.0841507000 |
| M84739 | Autoantigen calreticulin | 2.9484496000 |
| U45878 | Inhibitor of apoptosis protein 1; Also: L49432 | −2.5991459000 |
| U22398 | Cdk-inhibitor p57KIP2 (KIP2) | −2.5926486000 |
| HG3033-HT3194 | Spliceosomal protein Sap 62 | 2.7255114000 |
| J04794 | Aldehyde reductase | −2.5857708000 |
| M24486 | Prolyl 4-hydroxylase alpha subunit; Also: M24487, U14620_1 | 2.4710569000 |
| X74570 | Gal-beta(1-3/1-4)GlcNAc alpha-23-sialyltransferase | −2.5341531000 |
| D64142 | Histone H1x | −2.5299434000 |
| M21904 | 4F2 glycosylated heavy chain (4F2HC) antigen | 2.4384632000 |
| S68616 | Na+/H+ exchanger NHE-1 isom | −2.5223464000 |
| AB002318 | KIAA0320 | −2.4959950000 |
| M27826 | Endogenous retroviral protease | −2.4913967000 |
| HG2614-HT2710 | Collagen Type Viii Alpha 1 | 2.3734545000 |
| U12707 | Wiskott-Aldrich syndrome protein (WASP); Also: U19927 | 2.3664510000 |
| *U37408 | CtBP | 2.3356885000 |
| X86691 | 218 kD Mi-2 protein | −2.4076458000 |
| U02619 | TFIIIC Box B-binding subunit | 2.2834708000 |
| X99142 | Hair keratin hHb6 | 2.2847916000 |
| D26561 | ORF E7 from papillomavirus 5b genome | −2.4064124000 |
| U35234 | Protein tyrosine phosphatase sigma | 2.2769785000 |
| D11086 | Interleukin 2 receptor gamma | −2.3887671000 |
| U22970 | 16-Jun (interferon-inducible peptide precursor); U22970 | −2.3853381000 |
| D31833 | Vasopressin V1b receptor; Also: L37112 | 2.2463878000 |
| S76475 | TrkC from brain | −2.3536759000 |
| D63486 | KIAA0152 | 2.2292978000 |
| M96326 | Azurocidin | −2.3481101000 |
| M91083 | DNA-binding protein (HRC1) | 2.2142609000 |
| U23803 | Heterologous ribonucleoprotein A0 | 2.2093005000 |
| X54637 | Tyk2 non-receptor protein tyrosine kinase | 2.2109202000 |
| J03600 | Lipoxygenase | −2.3282267000 |
| Z11502 | Intestine-specific annexin | 2.1989181000 |
| AB000895 | Cadherin FIB1 | −2.3181155000 |
| M12125 | Fibroblast muscle-type tropomyosin | −2.3107464000 |
| D49817 | Fructose 6-phosphate 2-kinase/fructose 2 6-bisphosphatase | 2.1814434000 |
| D42085 | KIAA0095 | −2.3021685000 |
| D49818 | Fructose 6-phosphate 2-kinase/fructose 2 6-bisphosphatase | 2.1724424000 |
| M79463 | PML-2; Also: HG560-HT560, M82827 | −2.2904798000 |
| U89336 | Notch 4 | −2.2881933000 |
| Y08409 | Spot14 | −2.2876898000 |
| S81737 | Alpha 1 syntrophin; Also: U40571 | 2.1638767000 |
| D10495 | Protein kinase C delta-type | 2.1579099000 |
| U48861 | Beta 4 nicotinic acetylcholine receptor subunit | 2.1518600000 |
| M97935 | Transcription factor ISGF-3 sequence; M97935 | −2.2592952000 |
| U37219 | Cyclophilin-like protein CyP-60 | −2.2589364000 |
| AC002077 | Cosmid clone LUCA17/3p213 | 2.1358956000 |
| Z78289 | (clone 1D2)/Z78289 | 2.1371687000 |
| D55640 | Monocyte pseudoautosomal boundary-like sequence | −2.2541249000 |
| U31383 | G protein gamma-10 subunit | −2.2559957000 |
| U15131 | p126 (ST5) | 2.1301085000 |
| U31875 | Hep27 protein | −2.2477278000 |
| X15331 | Phosphoribosylpyrophosphate synthetase; D00860 | 2.1267157000 |
| U65932 | Extracellular matrix protein 1 (ECM1) | −2.2467447000 |
| X67325 | p27 | −2.2399248000 |
| M38258 | Retinoic acid receptor gamma 1 | 2.1084635000 |
| D50855 | Calcium-sensing receptor; Also: U20760, U20759 | −2.2251800000 |
| HG2036-HT2090 | Stimulatory Gdp/Gtp Exchange protein C-Ki-Ras P21 | −2.2257614000 |
| U40223 | Uridine nucleotide receptor (UNR) | 2.1007323000 |
| U68723 | Checkpoint suppressor 1 | 2.1021763000 |
| M25322 | Granule membrane protein-140 | −2.2175497000 |
| M94345 | Macrophage capping protein | −2.2180100000 |
| U89335 | Notch 4 | −2.2188636000 |
| D78156 | RasGTPase activating protein | 2.0933341000 |
| M33882 | p78 protein | −2.2166936000 |
| X99728 | NDUFV3/X99728 | −2.2137169000 |
| ***D16480 | Mitochondrial enoylCoA hydratase | 2.0832338000 |
| Z49254 | L23-related | −2.2020794000 |
| Z26256 | L-type calcium channel/Z26256 | 2.0780216000 |
| X53683 | LAG-1 | −2.1972116000 |
| HG2175-HT2245 | Myosin, Heavy Polypeptide 10, Non-Muscle; Also: M69181 | 2.0730215000 |
| D28423 | Pre-splicing factor SRp20 | 2.0701672000 |
| D87078 | KIAA0235 | 2.0722131000 |
| HG162-HT3165 | Tyrosine Kinase Receptor Axl 2 | 2.0712927000 |
| L78833 | Lfp35 from BRCA1, Rho7 and vatl | 2.0703335000 |
| X17651 | Myf-4 myogenic determination factor | −2.1869563000 |
| X17254 | TRANSCRIPTION FACTOR Eryf1 | 2.0632771000 |
| HG3570-HT3773 | Protein Phosphatase Inhibitor Homolog | −2.1780412000 |
| U63336 | MHC Class I region proline rich protein | −2.1787612000 |
| X62573 | RNA Fc receptor TC9 | −2.1802693000 |
| M27161 | MHC class I CD8 alpha-chain (Leu-2/T8) | −2.1755118000 |
| M58597 | ELAM-1 ligand fucosyltransferase (ELFT) | −2.1774644000 |
| S78085 | PDCD2 = programmed cell death-2/Rp8 homolog | −2.1691599000 |
| D85939 | p97 homolog | 2.0528093000 |
| U12139 | Alpha1 (XI) collagen (COL11A1)/U12139 | 2.0572285000 |
| U83843 | HIV-1 Nef interacting protein (Nip7-1)/U83843 | 2.0546131000 |
| X99350 | HFH4 | 2.0568858000 |
| Z48519 | XG (clone RACE5)/Z48519; Also: Z48518 | 2.0560468000 |
| U48707 | Protein phosphatase-1 inhibitor | −2.1658376000 |
| U87964 | Putative G-protein (GP-1) | −2.1662080000 |
| U03270 | Centrin | −2.1609185000 |
| M96684 | Pur (pur-alpha) | 2.0450686000 |
| U77968 | Neuronal PAS1 (NPAS1) | 2.0448728000 |
| AF005037 | Secretory carrier membrane protein (SCAMP1) | −2.1531286000 |
| M68864 | ORF | −2.1544240000 |
| U24683 | Anti-B cell autoantibody IgM heavy chain variable VDJ region | 2.0397115000 |
| X86018 | MUF1 protein | 2.0422801000 |
| Z70220 | Unknown protein (clone ICRFp507O0882)/Z70220 | 2.0423786000 |
| HG1019-HT1019 | Serine Kinase Psk-H1 | −2.1511399000 |
| HG3934-HT4204 | G1 Phase-Specific | −2.1499116000 |
| L49169 | G0S3 | −2.1515997000 |
| D49490 | Disulfide isomerase-related protein | −2.1380657000 |
| D83783 | KIAA0192 | −2.1368790000 |
| U45328 | Ubiquitin-conjugating enzyme (UBE2I); Also: U31882 | 2.0229701000 |
| M80563 | CAPL protein | −2.1310570000 |
| M94167 | Heregulin-beta2 | 2.0207548000 |
| M63573 | Secreted cyclophilin-like protein (SCYLP) | 2.0167254000 |
| D14827 | Tax helper protein 1 | −2.1194209000 |
| L24774 | Delta3, delta2-CoA-isomerase; Also: Z25821_rna1, Z25820 | 2.0109336000 |
| V00551 | Alpha interferon | 2.0107027000 |
| X12433 | pHS1-2 with ORF homolog to membrane receptor proteins | −2.1131910000 |
| D42053 | KIAA0091 | 2.0053307000 |
| M58026 | NB-1 | 2.0055666000 |
| X93996 | AFX prot | 2.0044933000 |
| U65011 | Preferentially expressed antigen of melanoma (PRAME) | −2.1092410000 |
| D30755 | KIAA0113 | −2.1044871000 |
| S83308 | SOX5 = Sry-related HMG box | 2.0011494000 |
| Z18951 | Caveolin | 2.0024685000 |
| Z48314 | Apomucin; Also: U06711 | 1.9990217000 |
| HG2290-HT2386 | Calcitonin | −2.1007151000 |
| L42354 | Clone 48ES4/L42354 | −2.1001120000 |
| M28825 | Thymocyte antigen CD1a | −2.0982975000 |
| U40992 | Heat shock protein hsp40 homolog | 1.9974082000 |
| U83192 | Post-synaptic density protein 95 (PSD95) | 1.9959202000 |
| X70218 | Protein phosphatase X | 1.9929730000 |
| Y08263 | AAD14 protein | −2.0928083000 |
| S34389 | Heme oxygenase-2 [kidney 1627 nt] | −2.0891099000 |
| L43579 | Clone 110298/L43579; Also: L43575 | 1.9857183000 |
| M80647 | Thromboxane synthase | 1.9832428000 |
| Z31695 | 43 kDa inositol polyphosphate 5-phosphatase | 1.9831751000 |
| L38487 | Estrogen receptor-related protein (hERRa1) | −2.0839503000 |
| D50402 | NRAMP1 | 1.9797759000 |
| *M94250 | Retinoic acid inducible factor (MK) | 1.9788194000 |
| *X64072 | CD18; Also: M15395 | 1.9817507000 |
| X93499 | RAB7 prot | 1.9775636000 |
| J05582 | Pancreatic mucin; Also: J05581 | 1.9740898000 |
| S66431 | RBP2 = retinoblastoma binding protein 2 | 1.9738895000 |
| U04343 | CD86 antigen | −2.0770043000 |
| X62153 | P1 protein (P1.h); Also: D38073 | −2.0763673000 |
| X79066 | ERF-1 | −2.0739931000 |
| AB000409 | MNK1 | −2.0695756000 |
| J02947 | Extracellular-superoxide dismutase (SOD3); Also: U10116 | 1.9665406000 |
| M30185 | Cholesteryl ester transfer protein | −2.0591846000 |
| U31384 | G protein gamma-11 subunit | −2.0615467000 |
| M23294 | Beta-hexosaminidase beta-subunit (HEXB) | 1.9578227000 |
| U18235 | ATP-binding cassette protein (ABC2) HFBCD04 clone | 1.9605421000 |
| HG3731-HT4001 | Ig Heavy Chain, Vdjrc Regions L23566 | 1.9552306000 |
| M54951 | Atrial natriuretic factor | 1.9556397000 |
| U20428 | SNC19 sequence | 1.9529377000 |
| X63755 | High-sulphur keratin | 1.9523322000 |
| X79483 | ERK6 extracellular signal regulated kinase | 1.9481684000 |
| AF001294 | IPL | −2.0447357000 |
| U16031 | TRANSCRIPTION FACTOR IL-4 Stat | −2.0447357000 |
| X98833 | Zinc finger protein, Hsal1 | −2.0473722000 |
| J04948 | Alkaline phosphatase (ALP-1) | 1.9444086000 |
| X04898 | Apolipoprotein All | 1.9464474000 |
| X52896 | Dermal fibroblast elastin; Also: HG2994-HT4851 | 1.9470905000 |
| X56199 | XIST a (locus DXS399E) | 1.9455917000 |
| X90872 | gp25L2 protein | 1.9451524000 |
| HG4757-HT5207 | Oncogene MII-Af4, Fusion Activated; Also: L13773 | −2.0424771000 |
| U33839 | Potassium channel/U33839 | −2.0417873000 |
| X99140 | Hair keratin hHb5 | 1.9380942000 |
| D88155 | DNA for Ad4BP (SF-1); Also: U76388 | −2.0367287000 |
| M68840 | Monoamine oxidase A (MAOA) | 1.9351040000 |
| U58032 | Myotubularin related protein 1 (MTMR1)/U58032 | 1.9342459000 |
| X70683 | SOX-4 protein | 1.9370161000 |
| D82060 | Kidney histidine rich putative membrane protein | −2.0292823000 |
| J04056 | Carbonyl reductase | −2.0295867000 |
| S73813 | CD39 = lymphoid cell activation antigen | −2.0275535000 |
| AF002224 | E6-AP ubiquitin protein ligase 3A/promoter P1 | 1.9280628000 |
| U13369 | Ribosomal DNA repeating unit/U13369 | 1.9283702000 |
| M19888 | Small proline rich protein (sprl), clone 128 | −2.0258177000 |
| U63455 | Sulfonylurea receptor (SUR1) | −2.0264311000 |
| X69433 | Mitochondrial isocitrate dehydrogenase (NADP+) | −2.0251010000 |
| X13293 | B-myb | 1.9241500000 |
| X71490 | Vacuolar proton ATPase subunit D | 1.9232699000 |
| D84239 | IgG Fc binding protein | −2.0197392000 |
| L03840 | Fibroblast growth factor receptor 4 (FGFR4); Also: X57205 | −2.0213543000 |
| *U57971 | Calcium ATPase isoform 3x/a; Also: U60414 | −2.0217060000 |
| X68688 | ZNF33B; Also: D31763 | −2.0095571000 |
| X16260 | Inter-alpha-trypsin inhibitor subunit 3; Also: X69532_rna1 | 1.9108378000 |
| U05040 | FUSE binding protein | −2.0033528000 |
| U83303 | GCP-2 (granulocyte chemotactic protein-2) | −2.0065730000 |
| X96698 | D1075-like | −2.0043214000 |
| HG2188-HT2258 | Paired Box Hup1 | 1.9040931000 |
| M55621 | N-acetylglucosaminyltransferase I (GlcNAc-TI) | 1.9061195000 |
| *M85220 | Heavy chain disease IgA chain CH3 region | 1.9073097000 |
| M92424 | p53 associated MDM2 | −1.9992393000 |
| M76732 | HOX7; Also: M97676 | 1.8986155000 |
| M98447 | Keratinocyte transglutaminase | −1.9956352000 |
| U71364 | Serine protase inhibitor (P19) | −1.9973864000 |
| Y10514 | CD152 protein/Y10514; Also: Y10508 | −1.9929951000 |
| D88146 | UDP-galactose transporter 2 | 1.8931108000 |
| U00928 | Clone CE29 4.1 (CAC)n/(GTG)n repeat-containing | 1.8961954000 |
| U29615 | Chitotriosidase precursor | 1.8968843000 |
| L05188 | Small proline-rich protein 2 (SPRR2B) | −1.9881128000 |
| D13988 | Rab GDI | 1.8898058000 |
| M88282 | Tactile protein | −1.9848647000 |
| Z22548 | Thiol-specific antioxidant protein | −1.9789790000 |
| D87937 | Alpha(1,2)fucosyltransferase 5 | −1.9756311000 |
| X78338 | Synthetic adenovirus transformed retinal cell line MRP | −1.9770373000 |
| HG3502-HT3696 | Homeotic protein Hox54 | 1.8756978000 |
| U33920 | Clone lambda 5 semaphorin | 1.8739306000 |
| X82207 | Beta-centractin (PC3) | −1.9657895000 |
| U07882 | Delta opioid receptor | 1.8667303000 |
| Y07565 | RIN protein; Also: U71204 | 1.8655777000 |
| AB000462 | SH3 binding RES4-23A | −1.9614270000 |
| HG3921-HT4191 | Homeotic protein C6, Class I | −1.9577270000 |
| J03909 | Gamma-interferon-inducible protein (IP-30) | −1.9586833000 |
| K02882 | IGHD (Ig delta-chain); Also: K02882_2 | −1.9580858000 |
| U78180 | Sodium channel 2 (hBNaC2) | −1.9604708000 |
| U15197 | Histo-blood group ABO protein | 1.8577545000 |
| D88795 | Cadherin | −1.9559282000 |
| M21984 | (clone PWHTnT16) skeletal muscle Troponin T | −1.9555675000 |
| U37248 | Alpha-mannosidase (6A8) | −1.9553269000 |
| X66401 | LMP2 from TAP1, TAP2, LMP2, LMP7 and DOB | −1.9525503000 |
| Z29083 | 5T4 Oncofetal antigen | −1.9550862000 |
| M37075 | Embryonic/atrial myosin light chain (MLC-1-emb/A isoform) | 1.8536982000 |
| U32576 | Apolipoprotein apoC-IV (APOC4) | −1.9517017000 |
| HG4557-HT4962 | Small Nuclear Ribonucleoprotein U1, 1snrp | 1.8495730000 |
| L20859 | Leukemia virus receptor 1 (GLVR1) | 1.8480315000 |
| L35240 | Enigma | −1.9442358000 |
| M21389 | Keratin type II (58 kD) | −1.9454686000 |
| U07919 | Aldehyde dehydrogenase 6 | −1.9452223000 |
| U28687 | Zinc finger containing protein ZNF157 (ZNF157) | −1.9474337000 |
| U52154 | G protein-coupled inwardly rectifying K+ channel Kir34 | −1.9444827000 |
| X07695 | Cytokeratin 4 | −1.9460836000 |
| X89101 | Fas (Apo-1, CD95)/X89101; Also: X83493, X63717, X83492 | −1.9459607000 |
| L40393 | Clone S171 | 1.8411404000 |
| M59916 | Acid sphingomyelinase (ASM) | 1.8401061000 |
| M58603 | Nuclear factor kappa-B DNA binding subunit (NF-kappa-B) | −1.9367650000 |
| U02632 | Calcium-activated potassium channel | −1.9338668000 |
| L05500 | Fetal brain adenylyl cyclase | 1.8354686000 |
| L37199 | Clone cD24-1 Huntington's candidate region fragment | 1.8371147000 |
| S46622 | Calcineurin A catalytic subunit | 1.8343889000 |
| U09411 | Zinc finger protein ZNF 132 | −1.9281397000 |
| U37519 | Aldehyde dehydrogenase (ALDH8) | −1.9301847000 |
| U80184 | FLII | 1.8287565000 |
| L27071 | Tyrosine kinase (TXK) | 1.8251339000 |
| X94453 | Pyrroline 5-carboxylate synthetase | 1.8253937000 |
| D79985 | KIAA0163 | −1.9194703000 |
| M54968 | K-ras onco protein | −1.9203842000 |
| U05340 | p55CDC | −1.9212962000 |
| U38480 | Retinoid X receptor-gamma | −1.9189473000 |
| *M13241 | N-myc | 1.8188182000 |
| M13981 | Inhibin A-subunit | 1.8177636000 |

TABLE 10
Gene targets in heterogeneous MS spinal cord gray matter specimens.

| Probe sets | Gene descriptions | Up (+) or down (−) |
|---|---|---|
| U70063 | Acid ceramidase | + |
| X65644 | MBP-2 MHC binding protein 2 | + |
| X71129 | Electron transfer flavoprotein beta subunit | − |
| L07515 | Heterochromatin protein homolog (HP1) | − |
| U82671 | HSP1-A from cosmids from Xq28 | − |
| D49958 | Membrane glycoprotein M6 | + |
| X95404 | Non-muscle type cofilin | − |
| **D78014 | Dihydropyrimidinase related protein-3 | + |
| X05997 | Gastric lipase | − |
| M64108 | Udulin 1 | − |
| L48546 | Tuberin (TSC2) | − |
| AB000381 | GPI-anchored molecule-like, also D84290 | − |
| D89859 | Zinc finger 5 protein | − |
| *D10704 | Choline kinase | + |
| *X64072 | CD18; Also: M15395 | + |
| X13916 | LDL-receptor related protein | + |
| U37690 | RNA polymerase II subunit (hsRPB10) | − |
| U12404 | Csa-19 | − |
| D13900 | Mitochondrial short-chain enoyl-CoA hydratase | − |
| X62654 | ME491/CD63 antigen | + |
| U34605 | Retinoic acid- and interferon-inducible 58 K prot RI58 | − |
| M15395 | Leukocyte adhesion protein (LFA-1/Mac-1) beta subunit | + |
| U20530 | Bone phosphoprotein spp-24 precursor/U20530 | − |
| U49785 | D-dopachrome tautomerase | − |
| M76378 | Cysteine-rich protein (CRP) | − |
| L19437 | Transaldolase containing transposable element | − |
| X79683 | Z68155 and others | + |
| M34079 | HIV tat transactivator binding protein-1 (tbp-1) | − |
| U15197 | Histo-blood group ABO protein | + |
| X69433 | Mitochondrial isocitrate dehydrogenase (NADP+) | − |
| M58286 | TNF receptor; Also: M63121, M33294, X55313, M75866 | + |
| U58090 | Hs-cul-4A | + |
| U01317 | Beta-globin thalassemia from beta globin region | − |
| M92449 | Human LTR | − |
| U09564 | Serine kinase | − |
| U48437 | Amyloid precursor-like protein 1 | − |
| AC000099 | Cosmid g0771a003 | − |
| S95936 | Transferrin | − |
| D26598 | Proteasome subunit HsC10-II | − |
| D83243 | NPAT | − |
| U06452 | Melanoma antigen recognized by T-cells (MART-1) | − |
| U90716 | Cell surface protein HCAR | − |
| X78992 | ERF-2 | + |
| U82256 | Arginase type II | − |
| M35252 | CO-029 | − |
| J03068 | DNF1552 (lung) | + |
| M64231 | Spermidine synthase | − |
| HG1471-HT3923 | Transcription Factor Oct-1a/1b; Also: X13403 | − |
| L17131 | High mobility group protein HMG-I(Y) | + |
| U94332 | Osteoprotegerin (OPG) | − |
| U53204 | Plectin (PLEC1) | + |
| U32581 | Lambda/iota-protein kinase C-interacting protein | − |
| D78151 | 26S proteasome subunit p97 | − |
| U59877 | Low-Mr GTP-binding protein (RAB31); Also: U57091 | + |
| U49974 | Mariner2 transposable element/U49974 | − |
| X99687 | Methyl-CpG-binding protein 2 intron 2/X99687 | − |
| AB002356 | KIAA0358; Also: U77352 | + |
| M75106 | Prepro-plasma carboxypeptidase B | − |
| X99720 | TPRC | + |
| U58675 | Olfactory receptor cluster | + |
| L13800 | Liver expressed protein/L13800 | − |
| *D38583 | Calgizzarin | + |
| L10413 | Farnesyltransferase alpha-subunit | − |
| D82344 | NBPhox | − |
| D38555 | KIAA0079 | + |

TABLE 11
Gene targets in heterogeneous MS spinal cord white matter specimens

| Probe sets | Gene descriptions | Up (+) or down (−) |
|---|---|---|
| Y09321 | TAFII105 | − |
| M15465 | Pyruvate kinase type L; Also: D13243 | − |
| U16120 | Placental taurine transporter; Also: Z18956 | − |
| U34976 | Gamma-sarcoglycan | − |
| U53442 | p38Beta MAP kinase | − |
| HG3570-HT3773 | Protein Phosphatase Inhibitor Homolog | − |
| Z11793 | Selenoprotein P | − |
| Z48501 | Polyadenylate binding protein II/Z48501; Also: U68105 | + |
| D28532 | Renal Na+-dependent phosphate cotransporter | − |
| D28114 | Myelin-associated oligodendrocytic basic protein | − |
| X69908 | Mitochondrial ATP synthase c subunit (P2 form) | − |
| X92814 | Rat HREV107-like protein | − |
| M62958 | R degradation slow (RDS) | − |
| U40763 | Clk-associated RS cyclophilin CARS-Cyp; Also: X99717 | − |
| D00723 | Hydrogen carrier protein | − |
| D90282 | Carbamyl phosphate synthetase I | − |
| M73077 | Glucocorticoid receptor repression factor 1 (GRF-1) | − |
| D87953 | RTP | − |
| X15675 | pTR7 repetitive sequence/X15675 | − |
| AF009674 | Axin | − |
| U06698 | Neuronal kinesin heavy chain | − |
| AF000545 | Putative purinergic receptor P2Y10 | − |
| M20471 | Brain-type clathrin light-chain a | − |
| AB000468 | Zinc finger protein RES4-26 | − |
| X00368 | Prolactin 5/X00368 | − |
| X97074 | mRNS clathrin-associated protein | − |
| D90224 | gp34 | − |
| L11695 | Activin receptor-like kinase (ALK-5) | − |
| M26880 | Ubiquitin | − |
| X51602 | Flt receptor-related tyrosine kinase | − |
| D00763 | Proteasome subunit HC9 | − |
| D38498 | PMS5 (yeast PMS1 homolog) | − |
| U37690 | RNA polymerase II subunit (hsRPB10) | − |
| X12530 | B lymphocyte antigen CD20 (B1, Bp35); Also: X07203 | − |
| D13413 | Tumor-associated 120 kDa nuclear protein p120 | − |
| Y00264 | Amyloid A4 precursor of Alzheimer's disease | − |
| L19437 | Transaldolase containing transposable element | − |
| L77864 | Stat-like protein (Fe65) | − |
| Y10262 | EYA3/Y10262; Also: U81602 | − |
| X66534 | Soluble guanylate cyclase large subunit | − |
| X04706 | Homeobox (clone HHO.c13); Also: X17360_rna1 | − |
| D79205 | Ribosomal protein L39 | − |
| HG3495-HT3689 | Collagen Type Ix Alpha 1 | − |
| U28369 | Semaphorin V | − |
| U91618 | Proneurotensin/proneuromedin N | − |
| L20941 | Ferritin heavy chain | − |
| S59184 | RYK = related to receptor tyrosine kinase | − |
| X17622 | HBK2 potassium channel protein | − |
| X17025 | Homolog of yeast IPP isomerase | − |
| U83461 | Putative copper uptake protein (hCTR2)/U83461 | − |
| U37359 | MRE11 homolog hMRE11 | − |
| U13395 | Oxidoreductase (HHCMA56) | − |
| L07548 | Aminoacylase-1 (ACY1) | − |
| U19247 | Interferon-gamma receptor alpha chain | + |
| U11877 | Interleukin-8 receptor type B (IL8RB)/U11877 | − |
| M84605 | Putative opioid receptor | − |
| M90657 | Tumor antigen (L6) | − |
| M79462 | PML-1 | − |
| U28687 | Zinc finger containing protein ZNF157 (ZNF157) | − |
| M13929 | c-myc-P64; Also: HG3523-HT4900, HG3523-HT4899 | − |
| M64929 | Protein phosphatase 2A alpha subunit | + |
| HG2815-HT4023 | Myosin, Light Chain/U02629; Also: HG2815-HT1357 | − |
| Z50853 | CLPP | − |
| L43338 | Cadherin/L43338 | − |
| U48959 | Myosin light chain kinase (MLCK) | − |
| HG1614-HT1614 | Protein Phosphatase 1 Alpha Catalytic Subunit | − |
| D17716 | N-acetylglucosaminyltransferase V | − |
| Y00636 | Lymphocyte function associated antigen-3 (LFA-3) | − |
| M27826 | Endogenous retroviral protease | − |
| U32576 | Apolipoprotein apoC-IV (APOC4) | − |
| L47276 | Alpha topoisomerase/L47276; Also: L47277 | − |
| L14787 | DNA-binding protein | − |
| D63880 | KIAA0159 | − |
| U41060 | Breast cancer estrogen regulated LIV-1 protein (LIV-1) | − |
| M62762 | Vacuolar H+ ATPase proton channel subunit | − |
| D10922 | FMLP-related receptor (HM63)-Also: M84562 | − |
| M21984 | Skeletal muscle Troponin T | − |
| **HG1877-HT1917 | Myelin Basic protein; Also: M13577 | + |
| X13546 | Putative HMG-17 non-histone protein | + |
| HG2465-HT4871 | Dna-Binding protein Ap-2 3 | − |
| L03840 | Fibroblast growth factor receptor 4 (FGFR4); X57205 | − |
| U07151 | GTP binding protein (ARL3) | − |
| Z68129 | H-IDH NADH isocitrate dehydrogenase gamma | − |
| U69126 | FUSE binding protein 2 (FBP2); Also: U94832 | − |
| D50863 | TESK1 | − |
| AB002382 | KIAA0384 | − |
| X01703 | Alpha-tubulin (b alpha 1) | − |
| L35249 | Vacuolar H+-ATPase Mr 56,000 subunit (HO57); M60346 | + |
| L36818 | (clone 51C-3) 51C protein | − |
| L25119 | Mu opiate receptor (MOR1) | − |
| M12125 | Fibroblast muscle-type tropomyosin | − |
| X15822 | COX VIIa-L liver-specific cytochrome c oxidase | − |
| M97347 | Beta-1,6-N-acetylglucosaminyltransferase; L41415 | − |
| D87258 | Cancellous bone osteoblast serin protease | − |
| X95240 | Cysteine-rich secretory protein-3; Also: X94323 | − |
| D86962 | KIAA0207 | − |
| M57703 | Melanin concentrating hormone (MCH); Also: S63697 | − |
| X02874 | (2′-5′) oligo A synthetase E | − |
| L76159 | FRG1 | − |
| AB000220 | Semaphorin E | − |
| M99487 | Prostate-specific membrane Antigen | − |
| D85758 | DROER homolog | + |
| X54637 | Tyk2 non-receptor protein tyrosine kinase | + |
| U89335 | Notch 4 | − |
| X14684 | La protein C-terminal region; Also: X13697, M20328 | − |
| X99975 | hRTR/hGCNF protein | − |
| AF005043 | Poly(ADP-ribose) glycohydrolase (hPARG) | − |
| X76132 | DCC | − |
| U50527 | BRCA2 region, sequence CG018; Also: U57962 | − |
| X74330 | DNA primase (subunit p48) | − |
| M96326 | Azurocidin | − |
| X55889 | Ciliary neurotrophic factor 1 | − |
| D28791 | PIG-A | − |
| D78367 | K12 keratin | − |
| D79997 | KIAA0175 | − |
| U02082 | Guanine nucleotide regulatory protein (tim1) | − |
| AF001294 | IPL | − |
| X63629 | P cadherin | − |
| D82060 | Kidney histidine rich putative membrane protein | − |
| D14822 | CBFA2T1 | − |
| D38437 | DNA mismatch repair | − |
| M25322 | Granule membrane protein-140 | − |
| M84424 | Cathepsin E (CTSE) | − |
| *M54927 | Myelin proteolipid protein | − |
| X57351 | 1-8D from interferon-inducible family; HG1538-HT1538 | − |
| U00952 | Clone A9A2BRB7 (CAC)n/(GTG)n repeat-containing | + |
| D31766 | KIAA0060 | + |
| U03057 | Actin bundling protein (HSN) | − |
| U65932 | Extracellular matrix protein 1 (ECM1) | − |
| S87759 | Protein phosphatase 2C alpha | − |
| HG2059-HT2114 | Arrestin Beta 2 | − |
| X77584 | ATL-derived factor/thiredoxin | + |
| HG651-HT4201 | Adducin Alpha Subunit 2 | + |
| L10374 | (clone CTG-A4) sequence | − |
| X85785 | DARC | − |
| L77886 | Protein tyrosine phosphatase | − |
| HG1980-HT2023 | Tubulin, Beta 2 | − |
| U28963 | Gps2 (GPS2) | + |
| U78180 | Sodium channel 2 (hBNaC2) | − |
| X78136 | hnRNP-E2 | + |
| X73460 | Ribosomal protein L3 | + |
| X60188 | ERK1 protein serine/threonine kinase | − |
| M14058 | Complement C1r | − |
| D30756 | KIAA0049 | − |
| U66061 | TCRBC1 from germline T-cell receptor beta chain | − |
| X66436 | Hsr1 | − |
| X77753 | TROP-2 | − |
| U06233 | POU domain protein (Brn-3b) | − |
| D28915 | Hepatitis C-associated microtubular aggregate p44 | − |
| M94556 | mitochondrial specific ss-DNA binding protein | − |
| HG1019-HT1019 | Serine Kinase Psk-H1 | − |
| X66079 | Spi-B | + |
| D83243 | NPAT | − |
| HG846-HT846 | Cyclophilin-Related protein | − |
| X96753 | Melanoma-associated chondroitin sulfate proteoglycan | − |
| M13903 | Involucrin | + |
| M13232 | Factor VII serine protease precursor; Also: J02933 | − |
| **L40027 | Glycogen synthase kinase 3 | − |
| Y00757 | Polypeptide 7B2 | − |
| U22398 | Cdk-inhibitor p57KIP2 (KIP2) | − |
| L16464 | ETS onco (PEP1) | − |
| U63336 | MHC Class I region proline rich protein | − |
| M81601 | Transcription elongation factor (SII) | + |
| AB000114 | Osteomodulin | − |
| X83929 | Type 3 desmocollin; Also: D17427 | − |
| S73813 | CD39 = lymphoid cell activation antigen | − |
| U37219 | Cyclophilin-like protein CyP-60 | − |
| V00594 | Metallothionein from cadmium-treated cells; J00271 | − |
| D83542 | Cadherin-15 | − |
| M31520 | Ribosomal protein S24; Also: HG3214-HT3391 | − |
| U04343 | CD86 antigen | − |
| X06272 | Docking protein (signal recognition particle receptor) | + |
| U38964 | PMS2 related (hPMSR2); Also: D38502 | − |
| D31765 | KIAA0061 | − |
| D50477 | Membrane-type matrix metalloproteinase 3; D83646 | − |
| U51477 | Diacylglycerol kinase zeta | − |
| X69920 | Calcitonin receptor; Also: L00587 | − |
| *D90086 | Pyruvate dehydrogenase beta subunit | − |
| U41816 | C-1 | − |
| X05130 | Prolyl 4-hydoxylase beta subunit; Also: J02783 | + |
| AB000895 | Cadherin FIB1 | − |
| *D63135 | ETS-like 30 kDa prot | − |
| U50330 | Procollagen C-protase (pCP-2) | − |
| X89894 | Nuclear receptor | − |
| U01691 | Annexin V (ANX5) 5-UTR; Also: X12454, M18366 | + |
| U49957 | LIM protein (LPP); Also: U49968 | − |
| D21851 | KIAA0028 | − |
| HG4704-HT5146 | Glial Growth Factor 2 | − |
| D55640 | Monocyte pseudoautosomal boundary-like sequence | − |
| D63874 | HMG-1 | − |
| L23333 | Corticotropin releasing factor receptor; Also: X72304 | + |
| U15131 | p126 (ST5) | + |
| M83088 | Phosphoglucomutase 1 (PGM1) | − |
| L42379 | Bone-derived growth factor (BPGF-1) | − |
| AF005037 | Secretory carrier membrane protein (SCAMP1) | − |
| J02947 | Extracellular-superoxide dismutase (SOD3) | + |
| U12707 | Wiskott-Aldrich syndrome protein (WASP); Also: U19927 | + |
| Y10204 | CD77 protein/Y10204 | − |
| U19906 | Arginine vasopressin receptor 1 (AVPR1) | − |
| HG3175-HT3352 | Carcinoembryonic Antigen | − |
| X15306 | NF-H 1 | − |
| D63851 | Unc-18 homolog | − |
| X64707 | BBC1 | − |
| M87339 | Replication factor C 37-kDa subunit | + |
| X86809 | Major astrocytic phosphoprotein PEA-15 | − |
| J04164 | Interferon-inducible protein 27-Sep | − |
| D14878 | Protein D123 | − |
| M93311 | Metallothionein-III | − |
| U04520 | Type IV collagen a5 chain (COL4A5) | − |
| X71129 | Electron transfer flavoprotein beta subunit | − |
| L14837 | Tight junction (zonula occludens) protein ZO-1 | + |
| D16469 | ORF Xq terminal portion | − |
| L11372 | Protocadherin 43 | − |
| X02751 | N-ras | − |
| J03824 | Uroporphyrinogen III synthase | + |
| S67798 | PH-20 | − |
| U16031 | TRANSCRIPTION FACTORS IL-4 Stat | − |
| L43964 | (clone F-T03796) STM-2 | − |
| U79526 | Orphan G-protein coupled receptor Dez isoform a | − |
| L10413 | Farnesyltransferase alpha-subunit | − |
| HG3432-HT3621 | Fibroblast Growth Factor Receptor K-Sam | − |
| M57466 | MHC class II HLA-DP light chain; Also: M83664, X00532 | + |
| D63876 | KIAA0154 | − |
| U07794 | Tyrosine kinase (TXK) | − |
| X13839 | Vascular smooth muscle alpha-actin | − |
| X07290 | HF12 | − |
| HG2264-HT2360 | Atpase Ca2+ Transporting Plasma Membrane 1 6 | − |
| U11037 | Sel-1 like | + |
| D63475 | KIAA0109 | − |
| U44754 | PSE-binding factor PTF gamma subunit | − |
| L78132 | Prostate carcinoma tumor antigen (pcta-1) | − |
| D86977 | KIAA0224 | − |
| U83192 | Post-synaptic density protein 95 (PSD95) | + |
| M21142 | Guanine nucleotide-binding protein G-s-alpha-3; M21142 | + |
| L41067 | NF-AT4c | − |
| D26561 | ORF E7 from papillomavirus 5b genome | − |
| U59111 | Dermatan sulfate proteoglycan 3 (DSPG3) | − |
| HG2999-HT4756 | Thyroid Peroxidase; Also: M25715 | + |
| M62403 | Insulin-like growth factor binding protein 4 (IGFBP4) | − |
| AB000115 | Unknown protein | − |
| D90276 | CGM7 nonspecific cross-reacting antigen (NCA) | − |
| M76424 | Carbonic anhydrase VII (CA VII) | + |
| D31762 | KIAA0057 | − |
| L76568 | S26 from excision and cross link repair protein (ERCC4) | − |
| X57579 | Activin beta-A subunit (2); Also: J03634 | + |
| X02404 | Second calcitonin related peptide (CGRP) | − |
| Y10936 | Hypothetical protein downstream of DMPK and DMAHP | − |
| D13643 | KIAA0018 | − |
| AF005361 | Importin alpha 6 | − |
| U41737 | Pancreatic beta cell growth factor (INGAP)/U41737 | − |
| K01383 | Metallothionein-I-A | − |
| D80008 | KIAA0186 | − |
| AB002533 | Qip1 | − |
| U68135 | SCC-S1c expressed in squamous cell cancer/U68135 | − |
| X79353 | XAP-4 GDP-dissociation inhibitor | − |
| D50310 | Cyclin I | + |
| D15049 | Protein tyrosine phosphatase | − |
| U31930 | Deoxyuridine nucleotidohydrolase | − |
| M55543 | Guanylate binding protein isom II (GBP-2) | − |
| M31303 | Oncoprotein 18 (Op18) | − |
| U45328 | Ubiquitin-conjugating enzyme (UBE2I); Also: U31882 | + |
| L34657 | Platelet/endothelial cell adhesion molecule-1 (PECAM-1) | + |
| U33818 | Inducible poly(A)-binding protein | + |
| M16653 | Pancreatic elastase IIB | − |
| U63825 | Hepatitis delta antigen interacting protein A (dipA) | − |
| AB002318 | KIAA0320 | − |
| X66276 | Skeletal muscle C-protein; Also: X73114 | − |
| D13631 | KIAA0006; Also: D25304 | + |
| M91467 | Serotonin receptor (5HT1E) | − |
| Z56281 | Interferon regulatory factor 3 | − |
| M37238 | Phospholipase C; Also: X14034 | + |
| X81003 | HCG V | − |
| J00277 | Lambda-[SK2-T2, HS578T]; RS-[3, 4, 6]) c-Ha-ras1 | − |
| L42373 | Protein phosphatase 2A B56-alpha | − |
| J00210 | IFNA (interferon alpha-d) | − |
| HG3523-HT4899 | Proto-Oncogene C-Myc; Also: L00058, HG3523-HT4900 | + |
| AF001548 | 815A9.1 myosin heavy chain from chromosome 16 | − |
| L24774 | Delta3, delta2-CoA-isomerase; Also: Z25821 | + |
| Z48054 | Peroxisomal targeting signal 1 (SKL type) receptor | − |
| D13720 | LYK; Also: L10717 | − |
| D87453 | KIAA0264 | − |

TABLE 12
Gene targets found across all comparisons
of MS gray matter spinal cord tissues against
normal gray matter spinal cord tissues.*

| Probe Set | Gene description | Mean fold change | Mean P value |
|---|---|---|---|
| *M87789 | Hybridoma H210 | 3.0601579 | 0.000696592 |
| | anti-hepatitis A IgG | | |
| | V, C, CDR regions | | |
| *X64072 | CD18; Also: M15395 | 1.8796121 | 0.00822973 |
| M13207 | Granulocyte-macrophage | −2.063521 | 0.012103845 |
| | colony-stimulating | | |
| | factor (CSF1) | | |
| HG2709- | Serine/Threonine Kinase | 1.6333372 | 0.016343018 |
| HT2805 | | | |
| M16707 | Histone H4; clone FO108 | −1.9707812 | 0.019372876 |

*Mean-fold change refers to the mean of the log₁₀(ratio)-fold changes of all MS gray matter vs. normal gray matter comparisons for that gene; mean P value refers to the mean significance value of all MS gray matter vs normal gray matter comparisons for that gene-these parameters also apply to Tables 13-15.

TABLE 13
Gene targets found across all comparisons of MS white matter spinal cord
tissues against normal white matter spinal cord tissues.

| Probe Set | Gene description | Mean fold change | Mean P value |
|---|---|---|---|
| M84739 | Autoantigen calreticulin | 2.6243939 | 0.003487238 |
| AB000895 | Cadherin FIB1 | −2.3163955 | 0.0055741 |
| U12707 | Wiskott-Aldrich syndrome protein (WASP); Also: U19927 | 2.2626829 | 0.005747785 |
| *M63438 | Ig rearranged gamma chain , V-J-C region; Also: X96754 | 3.0521967 | 0.006442657 |
| U35234 | Protein tyrosine phosphatase sigma | 2.2824217 | 0.007725067 |
| U37219 | Cyclophilin-like protein CyP-60 | −2.2580722 | 0.007876164 |
| U45328 | Ubiquitin-conjugating enzyme (UBE2I); Also: U31882 | 2.2253883 | 0.008423851 |
| *M87789 | Hybridoma H210 anti-hepatitis A IgG V, C, CDR regions | 2.6095958 | 0.010125314 |
| J02947 | Extracellular-superoxide dismutase (SOD3) | 2.1364149 | 0.012132355 |
| X54637 | Tyk2 non-receptor protein tyrosine kinase | 2.0976451 | 0.012253421 |
| *U37408 | CtBP | 2.09237 | 0.014673944 |
| L24774 | Delta3, delta2-CoA-isomerase; Also: Z25821_rna1 | 2.0763149 | 0.014787034 |
| HG3033-HT3194 | Spliceosomal protein Sap 62 | 2.2991536 | 0.015744522 |
| U15131 | p126 (ST5) | 2.040288 | 0.016092199 |
| *X64072 | CD18; Also: M15395 | 2.0389044 | 0.018208883 |
| HG162-HT3165 | Tyrosine Kinase Receptor Axl 2 | 2.0291313 | 0.018333948 |
| X15331 | Phosphoribosylpyrophosphate synthetase subunit one | 2.0054747 | 0.018860938 |
| M96684 | Pur (pur-alpha) | 2.0364039 | 0.022166073 |
| M23294 | Beta-hexosaminidase beta-subunit (HEXB) | 1.9720603 | 0.024128352 |
| D42053 | KIAA0091 | 1.9407077 | 0.024673169 |
| U71364 | Serine protase inhibitor (P19) | −1.9973864 | 0.026455898 |
| U83192 | Post-synaptic density protein 95 (PSD95) | 1.8968397 | 0.026959056 |
| M21984 | (clone PWHTnT16) skeletal muscle Troponin T | −1.9555675 | 0.030792454 |

TABLE 14
Gene targets found across all comparisons of
MS vs. normal spinal cord tissues, including gray
and white matter comparisons.

| Probe Set | Gene description | Mean fold change | Mean P value |
|---|---|---|---|
| *M87789 | Hybridoma H210 | 2.809845633 | 0.005934771 |
| | anti-hepatitis A IgG | | |
| | V, C, CDR regions | | |
| *X64072 | CD18; Also: M15395 | 1.968107833 | 0.013773704 |

TABLE 15
MS target genes commonly altered across MS spinal cord white matter samples
that contain inflammatory cells and have evidence of demyelination.

| Probe set | Gene description | Mean fold change | Mean P value |
|---|---|---|---|
| *M63438 | Ig rearranged gamma chain, V-J-C region; Also: X96754 | 3.1649421 | 0.00019838 |
| *M87789 | Hybridoma H210 anti-hepatitis A IgG V, C, CDR regions | 2.6612373 | 0.001353321 |
| D26561 | ORF E7 from papillomavirus 5b genome | −2.4064124 | 0.003085073 |
| U22970 | 16-Jun (interferon-inducible peptide precursor) | −2.3853381 | 0.003506211 |
| M84739 | Autoantigen calreticulin | 2.3988885 | 0.005304639 |
| AB000895 | Cadherin FIB1 | −2.3152488 | 0.005469599 |
| U45328 | Ubiquitin-conjugating enzyme (UBE2I); Also: U31882 | 2.2473988 | 0.005664919 |
| U12707 | Wiskott-Aldrich syndrome protein (WASP); Also: U19927 | 2.236641 | 0.006397345 |
| J04948 | Alkaline phosphatase (ALP-1) | 2.2390081 | 0.007089837 |
| U37219 | Cyclophilin-like protein CyP-60 | −2.257496 | 0.0076284 |
| J02947 | Extracellular-superoxide dismutase (SOD3); U10116 | 2.1735344 | 0.008235838 |
| X67325 | p27 | −2.2399248 | 0.00837286 |
| U35234 | Protein tyrosine phosphatase sigma | 2.2315119 | 0.010160845 |
| L41143 | Expressed pseudo TCTA at t(1;3) translocation site | 2.2002311 | 0.010868089 |
| U12139 | Alpha1(XI) collagen (COL11A1)/U12139 | 2.1197746 | 0.010952425 |
| X54637 | Tyk2 non-receptor protein tyrosine kinase | 2.1068855 | 0.011617197 |
| HG3033-HT3194 | Spliceosomal protein Sap 62 | 2.3472607 | 0.012248234 |
| HG162-HT3165 | Tyrosine Kinase Receptor Axl 2 | 2.1075667 | 0.012419809 |
| *X64072 | CD18; Also: M15395 | 2.1152978 | 0.0127252 |
| HG3945-HT4215 | Phospholipid Transfer protein | 2.3308357 | 0.013077384 |
| U02619 | TFIIIC Box B-binding subunit | 2.2199388 | 0.013347355 |
| X95406 | Cyclin E | −2.1339379 | 0.013951429 |
| L24774 | Delta3, delta2-CoA-isomerase; Also: Z25821 | 2.0417176 | 0.015024809 |
| M28825 | Thymocyte antigen CD1a | −2.0982975 | 0.016299644 |
| *U37408 | CtBP | 2.0620477 | 0.01643724 |
| M23294 | Beta-hexosaminidase beta-subunit (HEXB) | 2.0583126 | 0.017046586 |
| X07315 | PP15 (placental protein 15) | 2.0088193 | 0.017057852 |
| D42053 | KIAA0091 | 2.00626 | 0.017700929 |
| U15131 | p126 (ST5) | 2.010507 | 0.018236373 |
| U15655 | Ets domain protein ERF | 1.9931435 | 0.020560907 |
| M61199 | Cleavage signal 1 protein | 1.981728 | 0.021409014 |
| X93996 | AFX protein | 2.050328 | 0.023466008 |
| X15331 | Phosphoribosylpyrophosphate synthetase subunit one | 1.926366 | 0.024012526 |
| Z26256 | L-type calcium channel | 1.9508756 | 0.02443943 |
| M36429 | Transducin beta-2 subunit; Also: M16538 | 1.9340099 | 0.024855746 |
| U71364 | Serine protase inhibitor (P19) | −1.9973864 | 0.025176509 |
| U83192 | Post-synaptic density protein 95 (PSD95) | 1.8899076 | 0.026517609 |
| U33920 | Clone lambda 5 semaphorin | 1.8937119 | 0.028148796 |
| U27333 | Alpha (1,3) fucosyltransferase (FUT6), major transcript I | 1.8809063 | 0.028243361 |
| M96684 | Pur (pur-alpha) | 1.9775652 | 0.028255093 |
| M21984 | Clone PWHTnT16 skeletal muscle Troponin T | −1.9555675 | 0.029442849 |

TABLE 16
Genes previously reported to be dysregulated in MS central nervous system
tissues.*

| Probe set | Gene description | Up (+) or down (−) |
|---|---|---|
| U89606 | Pyridoxal kinase | + |
| D10704 | Choline kinase | + |
| X05610 | Type IV collagen alpha-2 chain | + |
| M87789 | Hybridoma H210 anti-hepatitis A IgG V, C, CDR regions | + |
| U18919 | Chromosome 17q12-21 clone pOV-2 | + |
| U16031 | TRANSCRIPTION FACTOR IL-4 Stat | + |
| HG1595-HT4788 | Heterogeneous Nuclear Ribonucleoprotein I; HG1595-HT4789 | + |
| U21090 | DNA polymerase delta small subunit | + |
| L26339 | Autoantigen | + |
| U62317 | Hypothetical protein 384D8_7 | + |
| D38583 | Calgizzarin | + |
| M13241 | N-myc | + |
| L25270 | XE169 | + |
| U37408 | CtBP | + |
| M63438 | Ig rearranged gamma chain, V-J-C region; Also: X96754 | + |
| L05624 | MAP kinase kinase; Also: L11284 | + |
| U52518 | Grb2-related adaptor protein (Grap) | + |
| U64573 | Connexin43 gap junction prot (connexin43)/U64573 | + |
| M35999 | Platelet glycoprotein IIIa (GPIIIa) | + |
| X13334 | CD14 myeloid cell-specific leucine-rich glycoprotein | + |
| M94250 | Retinoic acid inducible factor (MK) | + |
| M85220 | Heavy chain disease IgA chain CH3 region | + |
| X57809 | Rearranged Ig lambda light chain; S42404 | + |
| HG2730-HT2828 | Fibrinogen, A Alpha Polypeptide; Also: M58569 | + |
| D84145 | WS-3 | − |
| M11749 | Thy-1 glycoprotein | − |
| U28811 | Cysteine-rich fibroblast growth factor receptor (CFR-1) | − |
| S82024 | SCG10 = neuron-specific growth-associated protein/stathmin homolog | − |
| M74826 | Glutamate decarboxylase (GAD-2) | − |
| D90086 | Pyruvate dehydrogenase beta subunit | − |
| D61391 | Phosphoribosypyrophosphate synthetase-associated protein 39 | − |
| X00734 | Beta-tubulin (5-beta) with ten Alu family members | − |
| U57971 | Calcium ATPase isoform 3x/a; Also: U60414 | − |
| M54927 | Myelin proteolipid protein | − |
| X63578 | Parvalbumin | − |
| X07109 | Protein kinase C (PKC) type beta II | − |
| D63135 | ETS-like 30 kDa protein | − |
| J03263 | Lysosome-associated membrane glycoprotein (lamp A) | − |
| D13988 | Rab GDI | − |
| M58459 | Ribosomal protein (RPS4Y) isom | − |
| U60269 | Putative ERVK envelope protein | − |
| Y12711 | Putative progesterone binding protein | − |
| *These genes, denoted in Tables 1-15 by only one asterisk, are being shown for the purpose of (a) validation of the methodology of the present invention, and (b) for use as targets in combination with other genes found to be dysregulated by the inventors. The probe set identifiers | | |
| #for some of these genes (i.e., Ig lambda light chain and Heavy chain disease IgA chain CH3 region) were not mentioned in the report by Lock et al. (2002). The probe set identifier for fibrinogen (HG2730-HT2828) was not identical to the probe set in the report by Lock et al. (2002) (HG2730-HT2827). | | |

TABLE 17
Genes previously reported to be dysregulated
in MS brain lesions using high throughput
sequencing of cDNA libraries (Chabas et al.,
Science 2001; 294: 1731-5) and also shown
by the inventors to be similarly dysregulated in
MS spinal cords, using DNA microarrays.**

| | | Up (+) |
|---|---|---|
| Probe set | Gene Name | or Down (−) |
| D78014 | Dihydropyrimidinase related protein-3 | + |
| HG1877-HT1917 | Myelin Basic Protein | + |
| X05299 | Major centromere autoantigen CENP-B | + |
| L40027 | Glycogen synthase kinase 3 | − |
| **These genes, denoted in Tables 1-15 by two asterisks, are being shown for the purpose of (a) validation of the methodology of the present invention, and (b) for use as targets in combination with other genes found to be dysregulated by the inventors. Dihydropyrimidinase related protein- | | |
| #3 shares the probe set number identifier with the report by Chabas et al. (2001). The designation HG1877-HT1917 is a probe set identifier provided by Affymetrix. The probe set identifiers for Major centromere autoantigen CENP-B (X05299) and Glycogen synthase kinase 3 (L40027) are not identical to those reported by Chabas et al. for these genes. | | |

TABLE 18
Genes previously reported to be dysregulated in MS
brain lesions using filter cDNA microarrays
(Whitney et al., Annals of Neurology, 1999; 46(3): 425-8),
and also shown by the inventors to be similarly dysregulated
in MS spinal cords, using DNA microarrays.***

| Probe set | Gene Name | Up (+) or Down (−) |
|---|---|---|
| D16480 | Mitochondrial enoylCoA hydratase | + |
| J05037 | Serine dehydratase | + |
| ***These genes, denoted in Tables 1-15 by three asterisks, are being shown for the purpose of (a) validation of the methodology of the present invention, and (b) for use as targets in combination with other genes found to be dysregulated by the inventors. The gene identifiers | | |
| #reported by Whitney et al. (1999) (75860 for Mitochondrial enoylCoA hydratase and 76751 for serine dehydratase) differ from the probe set identifiers shown above. | | |

III. Gene Therapy

Thus, in accordance with the present invention, there are provided methods for the treatment of MS by gene therapy. Such methods include both the administration of a gene therapy vector encoding one or more genes identified as being downregulated in MS, and for genes that are overexpressed in MS, transgenes may be provided that reduce expression of appropriate targets.

MS is an inflammatory disease of the central nervous system. Entry of immune cells into the perivascular tissues of the brain and spinal cord lead to loss of myelin and eventually to axonal loss and neurodegeneration. In some cases, axonal loss occurs during the early stages of formation of the MS lesions. The molecules that mediate this tissue damage (i.e., from activated immune cells or from the affected tissues themselves) can be targeted with various therapies, to attempt to revert the ongoing demyelination or loss of axons. For instance, in Table 1, multiple genes are found to have elevated expression in MS tissues. Glutamate excitotoxicity has been linked to MS. Excitatory amino acid transporters and receptors, as well as calcium entry into the cells mediated by calcium channels, are mechanisms involved in glutamate toxicity. The metabotropic glutamate receptor 4 (probe set U92457) and the excitatory amino acid transporter 4 (U18244) are found to be elevated. Similarly, the P/Q-type calcium channel alpha 1 subunit was elevated. Among the inflammatory genes that are found to be elevated, complement component 2 (L09708), Ig-like transcript 2 (U82279), interleukin 8 receptor type B (U11877), Ig heavy chain VDJC region (HG4458-HT4727), monocyte chemoattractant protein-4 precursor (U46767), phospholipase A2 (M21056), granulocyte colony-stimulating factor receptor (CSF3R) (M59820) are found to be upregulated and would thus be considered targets for downregulation in MS. In contrast, the 5-HT6 serotonin receptor (L41147), and the STAT2 transcription factor (U18671) are downregulated, and therefore we propose that attempts to upregulate these transcripts will benefit MS patients. Similar examples of target selection can be extracted from Tables 2-10.

In Table 2, examples of target genes that are upregulated in MS spinal cords include the inflammation related genes Anti-hepatitis A IgG (*M87789) and the metal ion-related NRAMP1 (D50402). Downregulated genes that could be targeted for treating MS include the neurofilament triplet protein L (U57341), involved in neuronal cytoskeletal integrity, and a lymphoid specific transcription factor (M36542).

In Table 3, examples of target genes that are upregulated in MS spinal cords include the inflammation related genes CD14 (*X13334), C5a anaphylatoxin receptor (M62505), Fc receptor Iib3 for IgG (Affymetrix designation HG491-HT491), lymph node homing receptor (M25280), and the complement component properdin (M83652). Also in Table 3, downregulated genes that could be used as targets of treatment include guanylate kinase (L76200) involved in DNA repair, and mitochondrial creatine kinase (J04469) involved in energy metabolism.

In Table 4, elevated transcripts for the inflammation-related genes cathepsin C (X87212), T cell receptor zeta chain (J04132), and MHC class II HLA (M96132) may serve as targets of treatment. From Table 5, elevated phospholipid transfer protein (HG3945-HT4215), and downregulated transcripts for the DNA mismatch repair gene MLH1 (AF001359) and the glutamate transporter EAAT3 (U08989) are candidate targets. From Table 6, the downregulated TGF-beta superfamily protein (AB000584) may be used as a example. From Table 7, the repair gene Rad23A homolog (AD000092) was also downregulated. From Table 8, the apoptosis-related phospholipid scramblase (AF008445) is found to be an elevated target transcript. From Table 9, the inhibitor of apoptosis protein 1 (U45878) is downregulated, and elevation of its expression could prevent (neuronal) cell death. The immune attenuator CD152/CTLA4 (Y10514) is also downregulated, and elevation of its expression could attenuate inflammation in MS. The checkpoint suppressor 1 (U68723) is also found to be elevated. From Table 10, upregulated transcripts that are hereby proposed as targets of MS treatments include acid ceramidase (U70063), the MHC-binding protein 2 MBP2 (X65644), and choline kinase (*D10704). Also, from Table 11, decreased mitochondrial ATP synthase (X69908), and increased interferon γ (IFN-γ) receptor a chain (U19247) can be used as targets of treatment. A similar approach can be implemented for selecting genes from Tables 12 to 15.

The above genes are mentioned only by way of example, for a concept of treating MS that can be applied to all other genes in the list. Various aspects of gene delivery and expression are set forth below.

1. Therapeutic Transgenes

Thus, in accordance with the present invention, there are provided methods of treating and preventing MS utilizing genes identified as being overexpressed or underexpressed in MS, as illustrated in Tables 1-15. By inhibiting or increasing the expression of various of these genes, therapeutic benefit may be provided to patients.

2. Antisense

In contrast to “replacement” gene therapy, described above, it may be desirable to down-regulate the expression of certain targets that are overexpressed in individuals afflicted with, or at risk of, MS. A variety of mechanisms are available for effecting a decrease in gene expression using genetic constructs.

The term “antisense” nucleic acid refers to oligo- and polynucleotides complementary to bases sequences of a target DNA or RNA. When introduced into a cell, antisense molecules hybridize to a target nucleic acid and interfere with its transcription, transport, processing, splicing or translation. Targeting double-stranded DNA leads to triple helix formation; targeting RNA will lead to double helix formation.

Antisense constructs may be designed to bind to the promoter or other control regions, exons, introns or even exon-intron boundaries of a gene. Antisense RNA constructs, or DNA encoding such antisense RNA's, may be employed to inhibit gene transcription or translation within a host cell. Nucleic acid sequences which comprise “complementary nucleotides” are those which are capable of base-pairing according to the standard Watson-Crick complementarity rules. That is, that the larger purines will base pair with the smaller pyrimidines to form combinations of guanine paired with cytosine (G:C) and adenine paired with either thymine in the case of DNA (A:T), or uracil (A:U) in the case of RNA. Inclusion of less common bases such as inosine, 5-methylcytosine, 6-methyladenine, hypoxanthine and others in hybridizing sequences does not interfere with pairing.

As used herein, the terms “complementary” and “antisense sequences” mean nucleic acid sequences that are substantially complementary over their entire length and have very few base mismatches. For example, nucleic acid sequences of fifteen bases in length may be termed complementary when they have complementary nucleotides at thirteen or fourteen positions. Naturally, nucleic acid sequences which are “completely complementary” will be nucleic acid sequences which have perfect base pair matching with the target sequences, i.e., no mismatches. Other sequences with lower degrees of homology are contemplated. For example, an antisense construct with limited regions of high homology, but overall containing a lower degree (50% or less) total homology, may be used.

While all or part of the gene sequence may be employed in the context of antisense construction, statistically, any sequence of 17 bases long should occur only once in the human genome and, therefore, suffice to specify a unique target. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting a gene simply by testing the construct in vitro to determine whether the gene's function or expression is affected.

In certain embodiments, one may wish to employ antisense constructs which include other elements, for example, those which include C-5 propyne pyrimidines. Oligonucleotides which contain C-5 propyne analogs of uridine and cytidine have been shown to bind RNA with high affinity and to be potent inhibitors or gene expression. Wagner et al. (1993).

3. Ribozymes

The term “ribozyme” refers to an RNA-based enzyme capable of targeting and cleaving particular DNA and RNA sequences. Ribozymes can either be targeted directly to cells, in the form of RNA oligonucleotides incorporating ribozyme sequences, or introduced into the cell as an expression construct encoding the desired ribozymal RNA. Ribozymes may be used and applied in much the same way as described for antisense nucleic acids. Ribozyme sequences also may be modified in much the same way as described for antisense nucleic acids. For example, one could include modified bases or modified phosphate backbones to improve stability or function.

4. RNA Interference

RNA interference (RNAi) is a form of gene silencing triggered by double-stranded RNA (dsRNA). DsRNA activates post-transcriptional gene expression surveillance mechanisms that appear to function to defend cells from virus infection and transposon activity. Fire et al. (1998); Grishok et al. (2000); Ketting et al. (1999); Lin & Avery (1999); Montgomery et al. (1998); Sharp (1999); Sharp & Zamore (2000); Tabara et al. (1999). Activation of these mechanisms targets mature, dsRNA-complementary mRNA for destruction. RNAᵢ offers major experimental advantages for study of gene function. These advantages include a very high specificity, ease of movement across cell membranes, and prolonged down-regulation of the targeted gene. Fire et al. (1998); Grishok et al. (2000); Ketting et al. (1999); Lin & Avery (1999); Montgomery et al. (1998); Sharp (1999); Sharp & Zamore (2000); Tabara et al. (1999). Moreover, dsRNA has been shown to silence genes in a wide range of systems, including plants, protozoans, fungi, C. elegans, Trypanosoma and Drosophila. Grishok et al. (2000); Sharp (1999); Sharp & Zamore (1999).

Several principles are worth note (see Plasterk & Ketting, 2000) First, the dsRNA should be directed to an exon, although some exceptions to this rule have been shown. Second, a homology threshold (probably about 80-85% over 200 bases) is required. Most tested sequences are 500 base pairs or greater. Third, the targeted mRNA is lost after RNAᵢ. Fourth, the effect is non-stoichometric, and thus incredibly potent. In fact, it has been estimated that only a few copies of dsRNA are required to knock down >95% of targeted gene expression in a cell. Fire et al. (1998). Recently, shorter (˜20 base pairs) synthetic duplex RNAs have been shown to efficiently perform RNAi, by using liposome transfection. Further, similar short interfering RNA (siRNA) duplexes of 19-25 base pairs have been used by transfection via recombinant DNA constructs containing a promoter for U6 small nuclear RNA (snRNA) to drive nuclear expression of a single RNA transcript. This is also known as the hairpin siRNA/suppression of endogenous RNA (SUPER) strategy and has been shown to eliminate the expression of a target gene in long-term mammalian cell cultures (Brummelkamp et al., 2002; Paul et al., 2002; Lee et al., 2002; Miyagishi et al., 2002).

Although the precise mechanism of RNAᵢ is still unknown, the involvement of permanent gene modification or the disruption of transcription have been experimentally eliminated. It is now generally accepted that RNAᵢ acts post-transcriptionally, targeting RNA transcripts for degradation. It appears that both nuclear and cytoplasmic RNA can be targeted. Bosher and Labouesse (2000).

5. Single Chain Antibodies

Naturally-occurring antibodies (of isotype IgG) produced by B cells, consist of four polypeptide chains. Two heavy chains (composed of four immunoglobulin domains) and two light chains (made up of two immunoglobulin domains) are held together by disulphide bonds. The bulk of the antibody complex is made up of constant immunoglobulin domains. These have a conserved amino acid sequence, and exhibit low variability. Different classes of constant regions in the stem of the antibody generate different isotypes of antibody with differing properties. The recognition properties of the antibody are carried by the variable regions (VH and VL) at the ends of the arms. Each variable domain contains three hypervariable regions known as complementarity determining regions, or CDRs. The CDRs come together in the final tertiary structure to form an antigen binding pocket. The human genome contains multiple fragments encoding portions of the variable domains in regions of the immunoglobulin gene cluster known as V, D and J. During B cell development these regions undergo recombination to generate a broad diversity of antibody affinities. As these B cell populations mature in the presence of a target antigen, hypermutation of the variable region takes place, with the B cells producing the most active antibodies being selected for further expansion in a process known as affinity maturation.

A major breakthrough was the generation of monoclonal antibodies, pure populations of antibodies with the same affinity. This was achieved by fusing B cells taken from immunized animals with myeloma cells. This generates a population of immortal hybridomas, from which the required clones can be selected. Monoclonal antibodies are very important research tools, and have been used in some therapies. However, they are very expensive and difficult to produce, and if used in a therapeutic context, can elicit and immune response which will destroy the antibody. This can be reduced in part by humanizing the antibody by grafting the CDRs from the parent monoclonal into the backbone of a human IgG antibody. It may be better to deliver antibodies by gene therapy, as this would hopefully provide a constant localized supply of antibody following a single dose of vector. The problems of vector design and delivery are dealt with elsewhere, but antibodies in their native form, consisting of two different polypeptide chains which need to be generated in approximately equal amounts and assembled correctly are not good candidates for gene therapy. However, it is possible to create a single polypeptide which can retain the antigen binding properties of a monoclonal antibody.

The variable regions from the heavy and light chains (VH and VL) are both approximately 110 amino acids long. They can be linked by a 15 amino acid linker (e.g., (glycine₄serine)₃), which has sufficient flexibility to allow the two domains to assemble a functional antigen binding pocket. Addition of various signal sequences allows the scFv to be targeted to different organelles within the cell, or to be secreted. Addition of the light chain constant region (Ck) allows dimerization via disulphide bonds, giving increased stability and avidity. However, there is evidence that scFvs spontaneously multimerize, with the extent of aggregation (presumably via exposed hydrophobic surfaces) being dependent on the length of the glycine-serine linker.

The variable regions for constructing the scFv are obtained as follows. Using a monoclonal antibody against the target of interest, it is a simple procedure to use RT-PCR to clone out the variable regions from mRNA extracted from the parent hybridoma. Degenerate primers targeted to the relatively invariant framework regions can be used. Expression constructs are available with convenient cloning sites for the insertion of the cloned variable regions.

6. Vectors

In accordance with the present invention, both stimulatory and inhibitory genes may be provided to cells of an MS patient and expressed therein. Stimulatory genes are generally simply copies of the gene of interest, although in some cases they may be genes, the expression of which direct the expression of the gene of interest. Inhibitory genes, discussed above, may include antisense or single-chain antibody genes.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1989 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 19 lists non-limiting examples of elements/promoters that may be employed, in the context of the present invention, to regulate the expression of a RNA. Table 20 provides non-limiting examples of inducible elements, which are regions of a nucleic acid sequence that can be activated in response to a specific stimulus.

TABLE 19
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et |
| | al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn et |
| | al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek et |
| | al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake et |
| | al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et |
| | al., 1988; Feng et al., 1988; Takebe et al., 1988; Rosen et |
| | al., 1988; Berkhout et al., 1989; Laspia et al., 1989; Sharp |
| | et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 20
Inducible Elements

| Element | Inducer | References |
|---|---|---|
| MT II | Phorbol Ester (TFA) | Palmiter et al., 1982; Haslinger et |
| | Heavy metals | al., 1985; Searle et al., 1985; |
| | | Stuart et al., 1985; Imagawa et |
| | | al., 1987, Karin et al., 1987; |
| | | Angel et al., 1987b; McNeall et |
| | | al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai et |
| | | al., 1988 |
| β-Interferon | Poly(rI)x | Tavernier et al., 1983 |
| | Poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T | Taylor et al., 1989, 1990a, 1990b |
| | Antigen | |
| Proliferin | Phorbol Ester-TPA | Mordacq et al., 1989 |
| Tumor Necrosis Factor α | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Non-limiting examples of such regions include the human LIMK2 gene (Nomoto et al., 1999), the somatostatin receptor 2 gene (Kraus et al., 1998), murine epididymal retinoic acid-binding gene (Lareyre et al., 1999), human CD4 (Zhao-Emonet et al., 1998), mouse α2 (XI) collagen (Tsumaki et al., 1998), D1A dopamine receptor gene (Lee et al., 1997), insulin-like growth factor II (Wu et al., 1997), and human platelet endothelial cell adhesion molecule-1 (Almendro et al., 1996). Of particular interest are the neuronal promoter NSE (neuron-specific enolase), and the glial promoter GFAP (glial fibrillary acidic protein).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference).

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated.

A terminator may be necessary in vivo to achieve desirable message levels. In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392. Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

j. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system as it has a high frequency of integration and it can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding gene of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe. Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

7. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel and Baltimore, 1987), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

a. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

b. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

c. Calcium Phosphate

In other embodiments of the present invention, a nucleic acid may be introduced to the cells using calcium phosphate precipitation in an ex vivo context. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

d. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

e. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

f. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is a nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

g. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, has been incorporated into liposomes and an increase in the uptake of the insulin gene by hepatocytes has been observed (Nicolau et al., 1987). It is contemplated that the tissue-specific transforming constructs of the present invention can be specifically delivered into a target cell in a similar manner.

h. Microprojectile Bombardment

Microprojectile bombardment techniques can be used to introduce a nucleic acid ex vivo into at least one, organelle, cell, or tissue (U.S. Pat. Nos. 5,550,318, 5,538,880, 5,610,042, and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

IV. Methods of Assaying for Alterations in Gene Expression

In accordance with the present invention, methods are provided for assessing the ability of a genetic construct to alter the expression of target genes in patients suffering from or at risk of MS. In each of these assays, the expression of one or more genes, identified in Tables 1-15, will be measured. Genes that play a role in the immune system can be targeted to, and measured from the peripheral blood cells, or alternatively, targeted to, and measured directly from, the inflammatory lesions (i.e., when lesions are biopsied to rule out tumors). Non-immune genes (for instance, genes associated with neurodegeneration or demyelination) can be targeted to, and measured from (if biopsy is done), the brain or spinal cord lesions, regardless of the presence or absence of inflammation. The following is a discussion of various aspects of these methods.

1. Hybridization

There are a variety of ways by which one can assess gene expression. These methods either look at protein or at mRNA levels. Methods looking at mRNAs all fundamentally rely, at a basic level, on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

2. Amplification of Nucleic Acids

Since many nucleic acids, especially mRNAs, are in low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemilluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously (Chamberlan et al., 1990). The presence of many PCR primers in a single tube could cause many problems, such as the increased formation of misprimed PCR products and “primer dimers,” the amplification discrimination of longer DNA fragment and so on. Normally, MPCR buffers contain a Taq Polymerase additive, which decreases the competition among amplicons and the amplification discrimination of longer DNA fragment during MPCR. MPCR products can further be hybridized with gene-specific probe for verification. Theoretically, one should be able to use as many as primers as necessary. However, due to side effects (primer dimers, misprimed PCR products, etc.) caused during MPCR, there is a limit (less than 20) to the number of primers that can be used in a MPCR reaction. See also European Application No. 0 364 255 and Mueller & Wold (1989).

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[α-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC. In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA or total RNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only detected but quantified, by which is meant that the measured signal from each hybridization spot is compared to a unit value from the signal emitted by a known number of end-labeled target nucleic acids to obtain an absolute count of the copy number.

V. Pharmaceutical Formulations and Routes of Administration

It will be necessary to prepare pharmaceutical compositions in a form appropriate for use in vivo. Generally, this will entail preparing gene therapy vectors that are essentially free of pyrogens, as well as other impurities that could be harmful to humans or animals.

The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

Administration of these compositions according to the present invention will be via any common route so long as the target tissue is available via that route. This includes intradermal, subcutaneous, intramuscular, intraperitoneal or intravenous injection. Such compositions would normally be administered as pharmaceutically acceptable compositions, described supra.

The active compounds also may be administered intranasally, intraalveolarly (inhaled), parenterally, intrathecally (into the spinal fluid compartment), intraparenchymally (into the brain or spinal cord tissues) or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy administration by a syringe is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients that may include water, binders, abrasives, flavoring agents, foaming agents, and humectants.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

RNA Isolation and Hybridization.

All samples were obtained from local or national tissue banks and were processed under full compliance with institutional IRB requirements. Recorded tissue collection times ranged from 2-15 hours post-mortem, with the averages being 4 hours for MS and 10 hours for normals. For DNA microarray analysis, total RNA was obtained from postmortem gray or white matter of lumbar spinal cord from 9 individuals (5 with MS, and 4 controls without neurologic disease, of which 2 white matter control samples yielded sufficient RNA for the study). To assess the gene expression profile of post-mortem MS spinal cords, the inventors compared 4 MS gray matter tissues to 4 gray matter controls and 5 MS white mater tissues to 2 white matter controls (Table 1).

Analysis of the MS spinal cord samples by histopathology of both gray and white matter (average autolysis time 3.2 h), using Luxol Fast Blue, Bodian, H&E stains and GFAP, lymphocyte common antigen (LCA) and CD68 immunostaining revealed sample heterogeneity with findings that ranged from little involvement to frank inflammation, demyelination, or axonal loss. The inventors isolated RNA directly from frozen spinal cord specimens using the Qiagen RNAeasy™ kit, following the manufacturer's recommendations. Briefly, frozen tissues were dissected to separate gray and white matter, followed by mortar fragmentation in the presence of liquid nitrogen. Total RNA was isolated using the Qiagen Rneasy™ kit with the modification that the upper lipid layer was entirely removed from all samples after resuspension and spinning with buffer RLT. Although this strategy led to the discard of approximately 20-40% of the RNA sample, it preserved the overall integrity of our RNA because the lipid layer is known to compromise RNA stability and quality. All RNA samples included met quality/control standards (i.e., 260/280 OD ratios>1.8 in all RNA samples, and ratio of 3′ to 5′ signal>1.7 for GAPDH and β-actin on GeneChip for RNA samples used in microarray experiments) and were used for microarray hybridization. Though it is recognized that post-mortem changes in levels of individual mRNAs presumably occurred, these were likely similar among the specimens.

The inventors then performed microarray hybridization using RNAs isolated from gray and white matter from these spinal cords, using Affymetrix protocols. DNA microarray analysis was performed using Affymetrix HuFL GeneChip probe arrays. These microarrays contain 7,070 distinct probe sets, representing approximately 6,800 human genes. Briefly, using 7-10 μg of total RNA, double-stranded cDNA was synthesized using the Superscript Choice System (Life Technologies) with the following modifications. In the first strand synthesis, the reverse transcription reaction contained a T7-(dT)₂₄ primer plus 0.1 M DTT and 10 mM dNTP mix. For second strand synthesis, E. coli DNA ligase (10 U/μl) and T4 DNA Polymerase I (10 U/μl), 10 mM dNTP mix and RNase H (2 U/μl) were used. Phenol-chloroform extraction was followed by in vitro transcription (IVT) (Ambion T7 Megascript System) with biotin labeling. IVT was performed with (1:3) biotinylated: unlabeled CTP and UTP. The Ambion T7 enzyme mix and T7 transcription buffer were added to the ds cDNA and NTP labeling mix (ATP, CTP, UTP, GTP, Bio-11-CTP and Bio-16-UTP). The NTP labeling mix was incubated for 5 hr at 37° C., and cleaned using RNeasy columns (Qiagen). Thirteen to 20 μg of fluorescently-labeled and chemically-fragmented cRNA were used for array hybridization. Fragmented cRNA and herring sperm DNA were added to the hybridization buffer containing 1.0 M NaCl, 10 mM Tris-HCL pH 7.6, and 0.01% Triton X-100.

The hybridization mixture was heated to 99° C. for 5 min., spun, and incubated at 45° C. for 5 min, and injected into the probe array cartridge. Hybridizations were carried out at 45° C. for 16 hours with mixing at 60 rpm. Following hybridization, solutions were removed, and arrays were rinsed and incubated with 0.1× ST-T (100 mM NaCl, 10 mM Tris-HCL pH 8.0, and 0.01% Triton X-100) at 50° C. for 20 min. Hybridized arrays were stained with 5.0 μg/ml streptavidin-phycoerythrin (Molecular Probes) and 2.0 mg/ml acetylated BSA (Sigma) in 1× ST-T at 40° C. for 15 min. The streptavidin-phycoerythrin step was repeated after an intermediate amplification step in which anti-streptavidin rabbit IgG antibodies and secondary biotinylated goat anti-rabbit antibodies are added to the samples. Following washes, probe arrays were scanned twice at 6 μm resolution using the GeneChip system confocal scanner.

Microarray Data Collection and Analysis.

Scanned image files were converted to mRNA expression levels using Affymetrix GeneChip3.1 software. This software assesses presence or absence of transcripts for each probe set, taking into account metrics such as background, noise, and comparison of intensities between Perfect Match (PM) and their control Mismatch (MM) probe cells. The average intensity of each microarray was scaled to a target intensity of 1500. Files containing the average difference intensity values (i.e., expression levels) for each probe set were imported into an Access database. We assigned an arbitrary minimal expression level (i.e., average difference) value of 20 to any negative or zero values prior to performing the statistical analysis.

Selection of Discriminatory Genes. Statistics.

Several statistical measures have been introduced to identify differentially expressed genes for two conditions (e.g., cancerous and normal tissues). Parametric tests such as P-value (Golub et al., 1999) and t-test (Thomas et al., 2001) are based on differences of group means, while non-parametric tests such as Wilcoxon rank sum (Mann-Whitney) test are based on differences of rank sums in groups (Thomas et al. 2001). A couple of measures such as Wilks' lambda were also proposed for the identification of discriminatory genes in multi-classes (Dudoit et al., 2001; Hwang et al., 2002). All these parametric and non-parametric statistical tests eventually use parametric distributions such as t-distribution or F-distribution. Thus, these tests may perform poorly, resulting in producing many “false positives” or even “false negatives,” due to violation of their underlying distribution assumptions. In many cases of t-test applications, it can be seen that a histogram of t-statistic values of all genes does not match with a t-distribution theoretically defined for a given number of array samples. This discrepancy between a real distribution (histogram) and a theoretical distribution leads to “false positives” or “false negatives.”

The inventors propose a novel method that employs a non-parametric empirical estimation of a distribution, kernel density estimator, for any statistical measure used to detect differential expressions of genes in two or multiple conditions (Wand and Jones, 1995). In this study, for each gene i, a log ratio (rᵢ) between expression levels of a MS sample (gᵢ^{MS}) and the mean of healthy samples (gᵢ^{H}) is calculated as a statistical measure, as shown in equation 1 (Lock et al. 2002).

$${r}_{i} = {log}_{10} ( \frac{{g}_{i}^{MS}}{{g}_{i}^{H}} ) \qquad ( 1 )$$

Then, an empirical distribution of those ratios, which can be used for the statistical hypothesis test to select differentially expressed genes, is obtained using kernel density estimator. The density for a certain ratio (r) is defined by n ratios (rᵢ) as shown in equation 2.

$$\hat{f} ( r ; h ) = {( nh )}^{- 1} {\sum }_{i = 1}^{n} K ( \frac{r - {r}_{i}}{h} ) \qquad ( 2 )$$

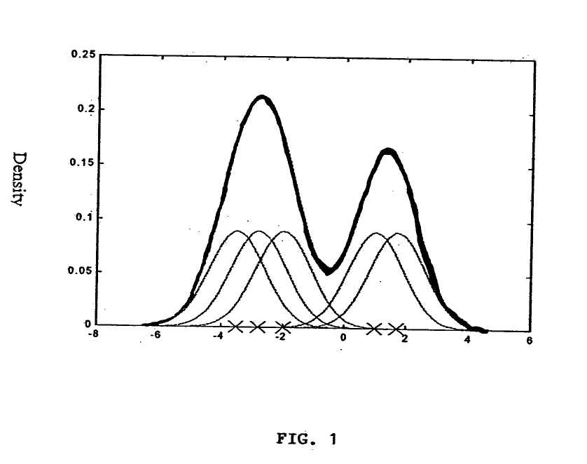

Here, K is a function satisfying ∫K(x)dx=1, which is called the kernel (normal distribution in this study), and h is a positive number, usually called the bandwidth or window width. FIG. 1 shows a kernel density estimate constructed using five ratios (five “x” marks on the x-axis) with a kernel chosen to be the normal distribution with zero mean and unit variance (N(0,1)).

In FIG. 1, the solid line is the kernel density estimated from the five ratios or kernels (dotted line) each of which is centered to each ratio. Just as a bin size, called the smoothing parameter, should be correctly chosen to explore the structure or shape of the distribution, the bandwidth should be optimally determined. A small bandwidth results in an under-smoothed estimate, while a large bandwidth an over-smoothed estimate. This sensitivity of the distribution shape to the bandwidth size is called variance-bias tradeoff. The optimal bandwidth is selected to minimize the mean integrated squared error (MISE) between the estimated density (\hat{ƒ}) and the target density (ƒ) for the kernel N(0,1), as shown in equation 3.

$$\hat{h} = {[ \frac{8 {π}^{1 / 2} R ( K )}{3 {{μ}_{2} ( K )}^{2} n} ]}^{1 / 5} s \qquad ( 3 )$$

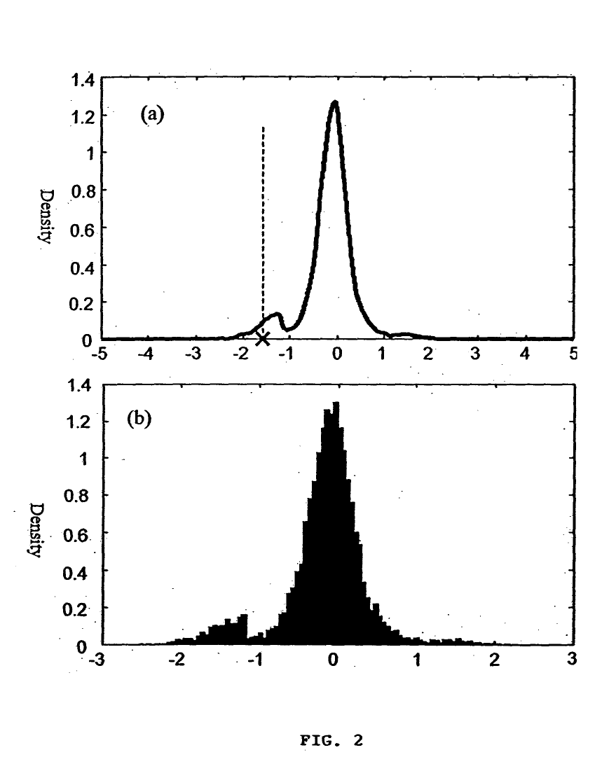

Here, R(K) and μ₂(K) are defined by ∫K(r)²dr and ∫r²K(r)dr, respectively, and s is the estimated standard deviation of ratios. Since this bandwidth to minimize MISE tends to be large, we tried ĥ/2, ĥ/4, and ĥ/8 to choose an optimal bandwidth among those derived from the MISE criterion in terms of the variance-bias tradeoff. In most of cases, ĥ/2 produced the optimal distribution structure (Wand and Jones, 1995). FIG. 2A shows a kernel density estimate for a MS sample where the optimal bandwidth is 0.052 and FIG. 2B shows a histogram with a bin size equivalent to the determined bandwidth. The kernel estimate indicates that there are two clear modes and one weak mode in the distribution. One clear mode in the left and one weak mode in the right of the distribution imply that there are more down-regulated genes in MS than up-regulated genes. Also, the mode in the center means that the majority of genes are not differentially expressed in MS and healthy patients. Comparing the kernel density estimate with the histogram, we can see that this estimated distribution matches well with the histogram.

With this empirically estimated distribution, a typical two-tailed hypothesis test is performed, as the typical t-test is done with t-distribution. The null hypothesis (H₀) is that means are equal and the alternative hypothesis (H₁) is that means are not equal. For instance, for a ratio marked on the x-axis in FIG. 2A, the area under the distribution below the ratio (aᵢ in equation 4) is calculated, and then the probability (called significance) of observing the given result by chance, when the null hypothesis is true, is determined as shown in equation 4.

pᵢ=2min(aᵢ,1−aᵢ) (4)

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

VII. References

The following references, to the extent that they provide exemplary procedural or other details supplementary to those set forth herein, are specifically incorporated herein by reference.

U.S. Pat. No. 3,817,837

U.S. Pat. No. 3,850,752

U.S. Pat. No. 3,939,350

U.S. Pat. No. 3,996,345

U.S. Pat. No. 4,275,149

U.S. Pat. No. 4,277,437

U.S. Pat. No. 4,366,241

U.S. Pat. No. 4,683,195

U.S. Pat. No. 4,683,202

U.S. Pat. No. 4,684,611

U.S. Pat. No. 4,797,368

U.S. Pat. No. 4,800,159

U.S. Pat. No. 4,883,750

U.S. Pat. No. 4,952,500

U.S. Pat. No. 5,139,941

U.S. Pat. No. 5,242,974

U.S. Pat. No. 5,279,721

U.S. Pat. No. 5,302,523

U.S. Pat. No. 5,322,783

U.S. Pat. No. 5,384,253

U.S. Pat. No. 5,384,261

U.S. Pat. No. 5,405,783

U.S. Pat. No. 5,412,087

U.S. Pat. No. 5,424,186

U.S. Pat. No. 5,429,807

U.S. Pat. No. 5,436,327

U.S. Pat. No. 5,445,934

U.S. Pat. No. 5,464,765

U.S. Pat. No. 5,472,672

U.S. Pat. No. 5,527,681

U.S. Pat. No. 5,529,756

U.S. Pat. No. 5,532,128

U.S. Pat. No. 5,538,877

U.S. Pat. No. 5,538,880

U.S. Pat. No. 5,545,531

U.S. Pat. No. 5,550,318

U.S. Pat. No. 5,554,501

U.S. Pat. No. 5,556,752

U.S. Pat. No. 5,561,071

U.S. Pat. No. 5,563,055

U.S. Pat. No. 5,571,639

U.S. Pat. No. 5,580,859

U.S. Pat. No. 5,589,466

U.S. Pat. No. 5,591,616

U.S. Pat. No. 5,593,839

U.S. Pat. No. 5,599,695

U.S. Pat. No. 5,610,042

U.S. Pat. No. 5,624,711

U.S. Pat. No. 5,656,610

U.S. Pat. No. 5,658,734

U.S. Pat. No. 5,700,637

U.S. Pat. No. 5,702,932

U.S. Pat. No. 5,736,524

U.S. Pat. No. 5,780,448

U.S. Pat. No. 5,789,215

U.S. Pat. No. 5,840,873

U.S. Pat. No. 5,843,640

U.S. Pat. No. 5,843,650

U.S. Pat. No. 5,843,651

U.S. Pat. No. 5,843,663

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,481

U.S. Pat. No. 5,849,486

U.S. Pat. No. 5,849,487

U.S. Pat. No. 5,849,497

U.S. Pat. No. 5,849,546

U.S. Pat. No. 5,849,547

U.S. Pat. No. 5,851,772

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,336

U.S. Pat. No. 5,882,864

U.S. Pat. No. 5,900,481

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,626

U.S. Pat. No. 5,919,630

U.S. Pat. No. 5,922,574

U.S. Pat. No. 5,925,517

U.S. Pat. No. 5,925,565

U.S. Pat. No. 5,928,862

U.S. Pat. No. 5,928,869

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,819

U.S. Pat. No. 5,935,825

U.S. Pat. No. 5,939,291

U.S. Pat. No. 5,942,391

U.S. Pat. No. 5,945,100

U.S. Pat. No. 5,981,274

U.S. Pat. No. 5,994,136

U.S. Pat. No. 5,994,624

U.S. Pat. No. 6,004,755

U.S. Pat. No. 6,013,516

U.S. Pat. No. 6,331,396

Abbondanzo, Ann. Diagn. Pathol., 3(5):318-327, 1999.

Allegreta et al., Science, 247(4943):718-21, 1990.

Allred et al., Arch Surg, 125(1):107-13, 1990.

Almendro et al., J Immunol., 157:5411-5421, 1996.

Alter et al., Proc. Nat'l Acad. Sci. USA, 97, 10101-10106 (2000).

Angel et al., Mol. Cell. Biol., 7:2256, 1987.

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Bagasra et al., Proc. Nat'l Acad. Sci. USA, 1995 Dec. 19;92(26):12041-5

Bagasra et al., Proc. Nat'l Acad. Sci. USA, 92(26):12041-5, 1995.

Baichwal and Sugden, In: Gene Transfer, Kucherlapati (ed.), New York, Plenum Press, 117-148, 1986.

Banerji et al., Cell, 27(2 Pt 1):299-308, 1981.

Banerji et al., Cell, 33(3):729-740, 1983.

Barcellos et al., Immunogenetics, 51: 281, 2000.

Berkhout et al., Cell, 59:273-282, 1989.

Blanar et al., EMBO J., 8:1139, 1989.

Blomer et al., J. Virol., 71(9):6641-6649, 1997.

Bo et al., Ann. Neurol., 36(5):778-86, 1994.

Bodine and Ley, EMBO J., 6:2997, 1987.

Boshart et al., Cell, 41:521, 1985.

Bosher and Labouesse, Nat. Cell Biol. 2(2):E31-36, 2000.

Bosze et al., EMBO J., 5(7):1615-1623, 1986.

Bowen et al., Clin. Pharmacol. Ther. 64, 339-46, 1998.

Braddock et al., Cell, 58:269, 1989.

Brown et al., Immunol Ser, 53:69-82, 1990.

Brummelkamp et al., Science 296, 550-553, 2002.

Bulla and Siddiqui, J. Virol., 62:1437, 1986.

Campbell and Villarreal, Mol. Cell. Biol., 8:1993, 1988.

Campere and Tilghman, Genes and Dev., 3:537, 1989.

Campo et al., Nature, 303:77, 1983.

Carbonelli et al., FEMS Microbiol Lett, 177(1):75-82, 1999.

Carlin et al., J. Neuropathol. Exp. Neurol. 59:361, 2000.

Celander and Haseltine, J. Virology, 61:269, 1987.

Celander et al., J. Virology, 62:1314, 1988.

Chamberlan et al., In: PCR Protocols, eds. Innis, Gelfand, Sninsky, White (Academic Press, NY, 272-281, 1990.

Chandler et al., Cell, 33:489, 1983.

Chang et al., Mol. Cell. Biol., 9:2153, 1989.

Chataway et al., Neurogenetics 2:91, 1999.

Chatterjee et al., Proc Nat'l Acad Sci. U.S.A., 86:9114, 1989.

Chen and Okayama, Mol. Cell Biol., 7(8):2745-2752, 1987.

Choi et al., Cell, 53:519, 1988.

Cocea, Biotechniques, 23(5):814-816, 1997.

Cohen et al., J. Cell. Physiol., 5:75, 1987.

Costa et al., Mol. Cell. Biol., 8:81, 1988.

Cotten et al., Proc Nat'l Acad Sci USA, 89(13):6094-6098, 1992.

Coupar et al., Gene, 68:1-10, 1988.

Cripe et al, EMBO J., 6:3745, 1987.

Culotta and Hamer, Mol. Cell. Biol., 9:1376, 1989.

Curiel, Nat. Immun., 13(2-3):141-64, 1994.

Dandolo et al., J. Virology, 47:55-64, 1983.

De Groot et al., J. Neuropathol. Exp. Neurol. 56(1):10-20, 1997.

De Jager et al., Semin. Nucl. Med. 23(2):165-179, 1993.

De Villiers et al., Nature, 312(5991):242-246, 1984.

Der et al., Proc. Nat'l Acad. Sci. USA 95:15623-8, 1998.

Deschamps et al., Science, 230:1174-1177, 1985.

Dillon and Goldstein, Multivariate Analysis, Wiley, New York; 1984.

Ding et al., J. Immunol., 160(6):2560-4, 1998.

Doolittle et al., Methods Mol Biol., 109:215-237, 1999.

Dudoit et al., U C Berkeley Statistics tech. Report, 2001.

Dudoit et al., Statistica Sinica 12, 111-139, 2002.

Edbrooke et al., Mol. Cell. Biol., 9:1908, 1989.

Edlund et al., Science, 230:912-916, 1985.

European Application No. 0 364 255

European Application No. 320 308

European Application No. EPO 0273085

European Application No. GB 2 202 328

European Application No. 329 822

Fechheimer et al., Proc. Nat'l Acad. Sci. USA, 84:8463-8467, 1987.

Feng and Holland, Nature, 334:6178, 1988.

Fernandez-Arquero et al. Neurology 53:1361, 1999.

Firak and Subramanian, Mol. Cell. Biol., 6:3667, 1986.

Fire et al., Nature, 391:806-811, 1998.

Fiten et al., J. Neuroimmunol. 95:195, 1999.

Foecking and Hofstetter, Gene, 45(1):101-105, 1986.

Fraley et al., Proc. Nat'l Acad. Sci. USA, 76:3348-3352, 1979.

Friedmann, Science, 244:1275-1281, 1989.

Frohman, In: PCR Protocols: A Guide To Methods And Applications, Academic Press, N.Y., 1990.

Fujita et al., Cell, 49:357, 1987.

Fukazawa et al., J. Neurol. Sci. 166: 47, 1999.

Fukazawa et al., J. Neurol. Sci. 171: 49, 1999.

Gade-Andavolu et al., Arch. Neurol. 55:513, 1998.

Ghosh and Bachhawat, In: Liver Diseases, Targeted Diagnosis and Therapy Using Specific Receptors and Ligands. Wu et al., eds., Marcel Dekker, New York, pp. 87-104, 1991.

Gilles et al., Cell, 33:717, 1983.

Giovannoni et al., J. Neurol. Sci., 145(1):77-81, 1997.

Gloss et al, EMBO J., 6:3735, 1987.

Godbout et al., Mol. Cell. Biol., 8:1169, 1988.

Golub et al., Science 286, 531-537, 1999.

Goodbourn and Maniatis, Proc. Nat'l Acad. Sci. USA, 85:1447, 1988.

Goodbourn et al., Cell, 45:601, 1986.

Gopal, Mol. Cell Biol., 5:1188-1190, 1985.

Graham and Van Der Eb, Virology, 52:456-467, 1973.

Greene et al., Immunology Today, 10:272, 1989

Grishok et al., Science, 287:2494-2497, 2000.

Grosschedl and Baltimore, Cell, 41:885, 1985.

Gulbis and Galand, Hum. Pathol. 24(12):1271-1285, 1993.

Harland and Weintraub, J. Cell Biol., 101:1094-1099, 1985.

Hashimoto et al., J. Neuroimmunol. 44:77, 1993.

Haslinger and Karin, Proc. Nat'l Acad. Sci. USA, 82:8572, 1985.

Hauber and Cullen, J. Virology, 62:673, 1988.

Hen et al., Nature, 321:249, 1986.

Hensel et al., Lymphokine Res., 8:347, 1989.

Herr and Clarke, Cell, 45:461, 1986.

Hintzen et al., JNI, 35:211-7, 1991.

Hirochika et al., J. Virol., 61:2599, 1987.

Hirsch et al., Mol. Cell. Biol., 10:1959, 1990.

Holbrook et al., Virology, 157:211, 1987.

Holter et al., Proc. Nat'l Acad. Sci. USA 97, 8409-8414, 2000.

Horlick and Benfield, Mol. Cell. Biol., 9:2396, 1989.

Horwich et al., Virol., 64:642-650, 1990.

Huang et al., Cell, 27:245, 1981.

Hug et al., Mol. Cell. Biol., 8:3065, 1988.

Hwang et al., Mol. Cell. Biol., 10:585, 1990.

Hwang et al., Bioinformatics, 18, 1184-1193, 2002.

Imagawa et al., Cell, 51:251, 1987.

Imbra and Karin, Nature, 323:555, 1986.

Imler et al., Mol. Cell. Biol, 7:2558, 1987.

Imperiale and Nevins, Mol. Cell. Biol., 4:875, 1984.

Innis et al., Proc. Nat'l Acad. Sci. USA 85(24):9436-9440, 1988.

Inouye and Inouye, Nucleic Acids Res., 13: 3101-3109, 1985.

Jacobsen et al., Nat. Genetics 26:495, 2000.

Jakobovits et al., Mol. Cell. Biol., 8:2555, 1988.

Jameel and Siddiqui, Mol. Cell. Biol., 6:710, 1986.

Jaynes et al., Mol. Cell. Biol., 8:62, 1988.

Jo et al., J. Biol. Chem., 276(19):16168-76, 2001.

Johnson and Applied Multivariate Statistical Analysis, Prentice Hall, New Jersey, 1992.

Johnson et al., Mol. Cell. Biol., 9:3393, 1989.

Johnston et al., Ann. Neurol., 50(4):434-42, 2001.

Kadesch and Berg, Mol. Cell. Biol., 6:2593, 1986.

Kaeppler et al., Plant Cell Reports 9: 415-418, 1990.

Kaneda et al., Science, 243:375-378, 1989.

Kantharia et al., Br. J. Rheumatol., 28(2):118-23, 1989.

Karin et al., Mol. Cell. Biol., 7:606, 1987.

Karin et al., Mol. Cell. Biol., 7:606, 1987.

Katinka et al., Cell, 20:393, 1980.

Kato et al., J. Biol. Chem., 266:3361-3364, 1991.

Kawamoto et al., Mol. Cell. Biol., 8:267, 1988.

Kelleher and Vos, Biotechniques, 17(6):1110-7, 1994.

Ketting et al., Cell, 99:133-141, 1999.

Kiledjian et al., Mol. Cell. Biol., 8:145, 1988.

Klamut et al., Mol. Cell. Biol., 10:193, 1990.

Klein et al., Nature, 327:70-73, 1987.

Knobeloch et al., Mol. Cell. Biol., 20(15):5363-9, 2000.

Koch et al., Mol. Cell. Biol., 9:303, 1989.

Kotze et al., Blood Cells Mol. Dis., 27(1):44-53, 2001.

Kraus et al., FEBS Lett., 428(3):165-170, 1998.

Kriegler and Botchan, In: Eukaryotic Viral Vectors, Y. Gluzman, ed., Cold Spring Harbor: Cold Spring Harbor Laboratory, N.Y., 1982.

Kriegler and Botchan, Mol. Cell. Biol., 3:325, 1983.

Kriegler et al., Cell, 38:483, 1984a.

Kriegler et al., Cell, 53:45, 1988.

Kuhl et al., Cell, 50:1057, 1987.

Kunz et al., Nucl. Acids Res., 17:1121, 1989.

Kwoh et al., Proc. Nat'l Acad. Sci. USA, 86: 1173, 1989.

Ladd et al., Mol. Cell Biol., 21(4):1285-96, 2001.

Lareyre et al., J. Biol. Chem., 274(12):8282-8290, 1999.

Larsen et al., Proc Nat'l Acad. Sci. USA., 83:8283, 1986.

Laspia et al., Cell, 59:283, 1989.

Latimer et al., Mol. Cell. Biol., 10:760, 1990.

Laughlin et al., J. Virol., 60(2):515-524, 1986.

Lawley et al., Ann. Rheum. Dis., 47(6):445-51, 1988.

Lebkowski et al., Mol. Cell. Biol., 8(10):3988-3996, 1988.

Lee et al., J. Auton. Nerv. Syst. 74(2-3):86-90, 1997.

Lee et al., Nature, 294:228, 1981.

Lee et al., Nucleic Acids Res., 12:4191-206, 1984.

Lee et al., Nat. Biotechnol. 19, 500-505, 2002.

Levenson et al., Hum Gene Ther, 9(8):1233-6, 1998.

Levinson et al., Nature, 295:79, 1982.

Lin and Avery, Nature, 402:128-129, 1999.

Lin et al., Mol. Cell. Biol., 10:850, 1990.

Liu et al. Am J Pathol, 158(6):2057-66, 2001.

Lock et al., Nature Medicine 8(5):500-508, 2002.

Lopez-Moratalla et al. Nitric Oxide, 1(1):95-104, 1997.

Lucotte et al., Mult. Scler. 6:78, 2000.

Luomala et al., Neurology 54:1862, 2000.

Luria et al., EMBO J., 6:3307, 1987.

Lusky and Botchan, Proc. Nat'l Acad. Sci. USA, 83:3609, 1986.

Lusky et al., Mol. Cell. Biol. 3:1108, 1983.

Macejak and Sarnow, Nature, 353:90-94, 1991.

Majors and Varmus, Proc. Nat'l Acad. Sci. USA, 80:5866, 1983.

Maniatis et al., Molecular Cloning, A Laboratory Manual, Cold Spring Harbor Press, Cold Spring Harbor, N.Y., 1989.

Mann et al., Cell, 33:153-159, 1983.

Mann et al., Neurology 54:552, 2000.

McLaughlin et al., J. Virol., 62(6):1963-1973, 1988.

McNeall et al., Gene, 76:81, 1989.

Miksicek et al., Cell, 46:203, 1986.

Miller et al., Am. J. Clin. Oncol., 15(3):216-221, 1992.

Miterski et al., Genes Immun. 1:37, 1999.

Miyagishi et al., Nat. Biotechnol. 19, 497-500, 2002.

Montgomery et al., Proc. Nat'l Acad. Sci. USA, 95:155-2-15507, 1998.

Mordacq and Linzer, Genes and Dev., 3:760, 1989.

Moreau et al., Nucl. Acids Res., 9:6047, 1981.

Mueller and Wold, Science 246, 780-786, 1989.

Muesing et al., Cell, 48:691, 1987.

Muller-Ladner et al., J. Neurol. Sci., 144(1-2):135-41, 1996.

Muzyczka, Curr Top Microbiol Immunol, 158:97-129, 1992.

Mycko et al., Ann. Neurol. 44:70, 1998.

Mycko et al., J. Neuroimmunol., 84:198, 1998.

Myhr et al., Neurology 52:1771, 1999.

Nabel and Baltimore, Nature 326:711-713, 1987.

Nakamura et al., In: Handbook of Experimental Immunology (4^{th} Ed.), Weir et al., (eds). 1:27, Blackwell Scientific Publ., Oxford, 1987.

Naldini et al., Science, 272(5259):263-267, 1996.

Ng et al., Nuc. Acids Res., 17:601, 1989.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, Biochim. Biophys. Acta, 721:185-190, 1982.

Nicolau et al., Methods Enzymol., 149:157-176, 1987.

Niino et al., J. Neurol. Sci. 179:70, 2000.

Nomoto et al., Gene, 236(2):259-271, 1999.

Ohara et al., Proc. Nat'l Acad. Sci. USA, 86: 5673-5677, 1989.

Omirulleh et al., Plant Mol. Biol., 21:415-28, 1993.

Ondek et al., EMBO J., 6:1017, 1987.

Omitz et al., Mol. Cell. Biol., 7:3466, 1987.

Padberg et al., J. Neuroimmunol., 99(2):218-23, 1999.

Palmiter et al., Nature, 300:611, 1982.

Paskind et al., Virology, 67:242-248, 1975.

Paul et al., Nat. Biotechnol. 20, 505-508, 2002.

PCT Application No. 95/06128

PCT Application No. WO 94/09699

PCT Application No. WO 94/09699

PCT Application PCT/US87/00880

PCT Application PCT/US89/01025

PCT Application WO 88/10315

PCT Application WO 90/07641

PCT Application WO 89/06700

Pech et al., Mol. Cell. Biol., 9:396, 1989.

Pelletier and Sonenberg, Nature, 334:320-325, 1988.

Perales et al., Proc. Nat'l Acad. Sci. USA, 91:4086-4090, 1994.

Perez-Stable and Constantini, Mol. Cell. Biol., 10:1116, 1990.

Picard and Schaffner, Nature, 307:83, 1984.

Pinkert et al., Genes and Dev., 1:268, 1987.

Plasterk and Ketting, Curr. Opin. Genet. Dev., 10:562-567, 2000.

Ponta et al., Proc. Nat'l Acad. Sci. USA, 82:1020, 1985.

Porton et al., Mol. Cell. Biol., 10: 1076, 1990.

Potrykus et al., Mol. Gen. Genet., 199:183-188, 1985.

Potter et al., Proc. Nat'l Acad. Sci. USA, 81:7161-7165, 1984.

Queen and Baltimore, Cell, 35:741, 1983.

Quinn et al., Mol. Cell. Biol., 9:4713, 1989.

Redondo et al., Science, 247:1225, 1990.

Reisman and Rotter, Mol. Cell. Biol., 9:3571, 1989.

Ren et al., Genes Dev., 16: 245-256, 2002.

Resendez Jr. et al., Mol. Cell. Biol., 8:4579, 1988.

Rice, Mathematical Statistics and Data Analysis, Duxbury Press, Belmont, Calif., 1995.

Rickert, Oncogene 12: 2631-40, 1996.

Ridgeway, In: Vectors: A survey of molecular cloning vectors and their uses, Stoneham: Butterworth, pp. 467-492, 1988.

Ripe et al., Mol. Cell. Biol., 9:2224, 1989.

Rippe et al., Mol. Cell Biol., 10:689-695, 1990.

Rittling et al., Nuc. Acids Res., 17:1619, 1989.

Rosen et al., Cell, 41:813, 1988.

Roux et al., Proc. Nat'l Acad. Sci. USA, 86:9079-9083, 1989.

Sakai et al., Genes and Dev., 2:1144, 1988.

Sambrook et al., In: Molecular Cloning: A Laboratory Manual, 2nd Ed., Cold Spring Harbor Press, Cold Spring Harbor, N.Y., 1989.

Sarchielli et al., J. Neuroimmunol., 80(1-2):76-86, 1997.

Satake et al., J. Virology, 62:970, 1988.

Schaffner et al., J. Mol. Biol., 201:81, 1988.

Searle et al., Mol. Cell. Biol., 5:1480, 1985.

Sharp and Marciniak, Cell, 59:229, 1989.

Sharp, Genes Dev., 13:139-141, 1999.

Sharpand Zamore, Science, 287:2431-2433, 2000.

Shaul and Ben-Levy, EMBO J., 6:1913, 1987.

Sherman et al., Mol. Cell. Biol., 9:50, 1989.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Statistical Toolbox, Math Works, Inc., Version 2.1.1, 1997.

Stelmasiak et al., Med. Sci. Monit., 7(5): 914-8, 2001.

Stephens and Hentschel, Biochem. J., 248:1, 1987.

Storey and Tibshirani, Stanford tech report, 2001.

Streit et al., J. Invest. Dermatol., 105(4):562-6, 1995.

Stuart et al., Nature, 317:828, 1985.

Sullivan and Peterlin, Mol. Cell. Biol., 7:3315, 1987.

Svenningsson et al., Neurology, 53(8):1880-2, 1999.

Swartzendruber and Lehman, J. Cell. Physiology, 85:179, 1975.

Tabara et al., Cell, 99:123-132, 1999.

Takebe et al., Mol. Cell. Biol., 8:466, 1988.

Takiguchi et al., Am. J. Physiol., 274(2 Pt 1):E265-70, 1998.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, Kucherlapati (ed.), New York: Plenum Press, pp. 149-188, 1986.

Thiesen et al., J. Virology, 62:614, 1988.

Thomas, Genome Research 11, 1227-1236, 2001.

Tienari et al., Lancet 340:987-91, 1992.

Tratschin et al., Mol. Cell. Biol., 4:2072-2081, 1984.

Treisman, Cell, 42:889, 1985.

Tronche et al., Mol. Biol. Med., 7:173, 1990.

Trudel and Constantini, Genes and Dev. 6:954, 1987.

Tsumaki et al., J. Biol. Chem. 273(36):22861-22864, 1998.

Tur-Kaspa et al., Mol. Cell Biol., 6:716-718, 1986.

Tusher et al., Proc. Nat'l Acad. Sci. USA 98, 5116-5121, 2001.

Two tailed t-test: www.psycho.uni-duesseldorf.de/aap/projects/gpower/reference/

Tyndell et al., Nuc. Acids. Res., 9:6231, 1981.

Vandenbroeck et al., Genes Immunol 1:460, 2000.

Vandenbroeck et al., J. Neuroimmunol. 76:189, 1997.

Vannice and Levinson, J. Virology, 62:1305, 1988.

Vasseur et al., Proc Nat'l Acad. Sci. U.S.A., 77:1068, 1980.

Wagner et al., Proc. Nat'l Acad. Sci. USA 87(9):3410-3414, 1990.

Wagner et al., Science, 260:1510-1513, 1993.

Walker et al., Proc. Nat'l Acad. Sci. USA, 89:392-396 1992.

Walter et al., J. Clin. Invest. 87:1266, 1991.

Wand and Jones, Kernel Smoothing. Chapman & Hall, 1995.

Wang and Calame, Cell, 47:241, 1986.

Warrington et al., Physiol. Genomics 2: 143-7, 2000.

Weber et al., Cell, 36:983, 1984.

Weinberger et al., Mol. Cell. Biol., 8:988, 1984.

Wilson et al., Science, 244:1344-1346, 1989.

Winer et al., J. Immunol., 166(7):4751-6, 2001.

Winoto and Baltimore, Cell 59:649, 1989.

Wong et al., Gene, 10:87-94, 1980.

Wu and Wu, J. Biol. Chem., 262:4429-4432, 1987.

Wu and Wu, Adv. Drug Delivery Rev., 12:159-167, 1993.

Wu and Wu, Biochemistry, 27:887-892, 1988.

Wu et al., Nature 414(6862):457-62, 2001.

Wu et al., Biochem Biophys Res Commun., 233(1):221-226, 1997.

Yamashita et al., J. Neurol. Sci., 153(1):32-4, 1997.

Yang et al., Proc. Nat'l Acad. Sci. USA, 87:9568-9572, 1990.

Yuceyar et al., Clin. Neurol. Neurosurg., 103(4):206-11, 2001.

Yusuf-Makagiansar et al., Med. Res. Rev. 22, 146-67, 2002.

Yutzey et al., Mol. Cell. Biol., 9:1397, 1989.

Zhao-Emonet et al., Biochem. Biophys. Acta., 1442(2-3):109-19, 1998.

Zufferey et al., Nat. Biotechnol., 15(9):871-875, 1997.

What is claimed is:

1. A method for treating or preventing multiple sclerosis (MS) comprising administering to a subject with MS a composition that causes an increase in the activity or expression of at least one gene product selected from the group consisting of those genes indicated by a minus (−) sign in Tables 1-15, other than those indicated by an asterisk.

2. The method of claim 1, further comprising a second MS therapy.

3. The method of claim 2, wherein the second MS therapy is given before the composition.

4. The method of claim 2, wherein the second MS therapy is given after the composition.

5. The method of claim 2, wherein the second MS therapy is given concurrent with the composition.

6. The method of claim 2, wherein said second MS therapy is interferon β1a, interferon β1b, glatiramer acetate, and mitoxantrone.

7. The method of claim 1, wherein the composition comprises peptide.

8. The method of claim 1, wherein the composition comprises a small molecule.

9. The method of claim 1, wherein the composition is an organo-pharmaceutical.

10. The method of claim 1, wherein the composition comprises an expression cassette comprising a nucleic acid encoding the selected gene product and a promoter active in eukaryotic cells, said nucleic acid operably linked to said promoter.

11. The method of claim 10, wherein said nucleic acid is comprised in a non-viral vector.

12. The method of claim 10, wherein said nucleic acid is comprised in a viral vector.

13. The method of claim 12, wherein said viral vector is an adenoviral vector, an adeno-associated viral vector, a retroviral vector, a herpesviral vector, a vaccinia viral vector or a polyoma viral vector.

14. The method of claim 10, wherein said promoter is a constitutive promoter, a tissue specific promoter or an inducible promoter.

15. The method of claim 14, wherein the tissue specific promoter is a neuronal cell promoter, a glial cell promoter, a monocyte promoter, a lymphocyte promoter, or a B cell promoter.

16. The method of claim 10, wherein said expression cassette further comprises a polyadenylation signal.

17. The method of claim 1, wherein said composition is administered intradermally, subcutaneously, intramuscularly, intraperitoneally, intravenously, intranasally, intraalveolarly, parenterally, intrathecally, intraparenchymally or intraperitoneally.

18. The method of claim 1, wherein said composition is administered to said mammal more than once.

19. The method of claim 1, wherein said composition is administered to said mammal in discrete repeated dosings.

20. The method of claim 1, wherein the at least one gene product comprises ORF E7 from papillomavirus 5b genome homolog (D26561), interferon-inducible peptide precursor 16-Jun (U22970), cadherin FIB1 (AB000895), cyclophilin-like protein Cyp-60 (U37219), and interferon α-inducible protein p27 (X67325).

21. The method of claim 1 or 20, wherein at least one gene product comprises cyclin E (X95406), thymocyte antigen CD1a (M28825), serine protease inhibitor p19 (U71364), and skeletal muscle troponin T (M21984).

22. The method of claims 20, or 21, wherein the at least one gene product further comprises IFNA (interferon alpha-d) (J00210), Interferon-alpha receptor (J03171), Interferon-inducible protein 27-Sep (J04164), Interferon regulatory factor 3 (Z56281), Interferon-induced 17-kDa/15-kDa protein (M13755), Interferon-inducible and MXA homolog p78 protein (M33882), Interferon-inducible 56 Kd protein (M24594), IFN-inducible 1-8D (X57351), Tumor necrosis factor type 1 receptor associated protein TRAP1 (U12595), granulocyte-macrophage colony stimulating factor CSF1 (M13207), TGF-beta superfamily protein (AB000584), Lipoxygenase (J03600), Myeloid cell differentiation protein (MCL1) (L08246), Chemokine TARC (D43767), CD110 protein (Y10506), CD152/CTLA4 (Y10514), Thrombospondin 2 (HG896-HT896), IgG Fc binding protein (D84239), Skin-antimicrobial-peptide 1 (SAP1) (Z71389), Mac-2 binding protein (L13210), Interleukin 1 receptor (M27492), Histone H1x (D64142), Histone H3.1 (M60746), Histone H4 (M16707), H4 histone (X60486), NBPhox (D82344), HOX2G (X16667), HOX4D (X59373), HOX11 (S38742), Androgen regulated homeobox protein (NKX3 1) (U80669), Paired-box protein (PAX2) (M89470), DNA-binding protein (GLI3) (M57609), Stat2 (U18671), Transcription factor ISGF-3 (M97935), Neurogenic basic-helix-loop-helix protein (NeuroD2) (U58681), H-neuro-d4 (U43843), Lim-Domain Transcription Factor Lim-1 (HG4318-HT4588), Cyclin D3 (M92287), Cyclin E1 (M74093), Cyclin G1 (X77794), Kinase Inhibitor P27kip1 Cyclin-Dependent (HG4258-HT4528), Cdk-inhibitor p57KIP2 (KIP2) (U22398), HsMcm6 (D84557), Retinoblastoma related protein (p107) (L14812), thymidylate synthase-inducer transcription factor LSF (U03494), 218 kD Mi-2 protein (X86691), Methyl-CpG-binding protein 2 (X99687), Androgen receptor (M23263), Retinoid X receptor beta (M84820), Orphan receptor ROR gamma (U16997), Orphan nuclear receptor (DAX1) (U31929), OTF-2 lymphoid-specific transcription factor (X13810), TRANSCRIPTION FACTOR (NFATcb) (U59736), NF-AT4c (L41067), YY1 (M77698), PLZF kruppel-like zinc finger protein (Z19002), Zinc finger protein, Hsa11 (X98833), TAFII70-alpha (L25444), RNA polymerase II associated protein RAP74 (X64037), AP-4 (S73885), Lupus p70 (Ku) autoantigen protein (J04611), Zinc Finger protein Znf155 (HG4243-HT4513), HPV16 E1 protein binding protein (U96131), E1A enhancer binding protein (E1A-F) (U18018), DNA-binding protein ABP/ZF (U82613), CCAAT transcription binding factor subunit gamma (NFY-C) (Z74792), anti-oxidant nuclear respiratory factor-1 (NRF-1) (L22454), Sodium Channel 1 (HG4593-HT4998), Sodium channel 2 (hBNaC2) (U78180), HBK2 potassium channel (X17622), Vacuolar H+ ATPase proton channel subunit (M62762), Calcium Channel, Voltage-Gated, Alpha 1e Subunit, 2 (HG3242-HT3419), Calcium Channel, Voltage-Gated, Alpha 1e Subunit, 3 (HG3242-HT4231), Beta nerve growth factor (X52599), Glial Growth Factor 2 (HG4704-HT5146), Fibroblast growth factor receptor 4 (L03840), Fibroblast Growth Factor Receptor K-Sam (HG3432-HT3621), Ciliary neurotrophic factor 1 (X55889), Heparin-binding EGF-like growth factor (M60278), Corticotropin releasing factor receptor (L23333), Connective tissue growth factor (M92934), Growth hormone releasing factor (L00137), Preprothyrotropin-releasing hormone (M63582), Proneurotensin-Proneuromedin N (U91618), Serotonin receptor (5HT1E) (M91467), 5-HT6 serotonin receptor (L41147), Serotonin 1B receptor (D10995), Calcitonin (HG2290-HT2386), Estrogen receptor-related protein (hERRa1) (L38487), m4 muscarinic acetylcholine receptor (M16405), ERF-1 (X79066), PTH-like hormone A (M24351), Dopamine DIA receptor (M85247), Dopamine D4 receptor (S76942), Glutamate transporter EAAT3 (U08989), Prepro-oxytocin-neurophysin I (OXT) (M11186), Vesicular acetylcholine transporter (U09210), Chorionic gonadotropin beta subunit (K03183), Enkephalin (J00123), Delayed rectifier potassium channel (KVLQTl-Iso5) (AF003743), Melanocortin 5 receptor (MC5R) (L27080), Thyroxine-binding globulin (M14091), GABA-A receptor, beta 1 subunit (X14767), Gamma-aminobutyric acid transporter type 3 (S75989), Strychnine binding subunit of inhibitory glycine receptor (X52008), EP3 prostanoid receptor EP3-I (S68874), Endomembrane proton pump subunit (M25809), NaK-ATPase gamma subunit (U50743), Mitochondrial ATP synthase c subunit (X69908), OXA1Hs (X80695), Electron transfer flavoprotein beta subunit (X71129), COX6B (AC002115), cytochrome c oxidase subunit VIIb (Z14244), Cytochrome b pseudo (U38268), Putative copper uptake protein hCTR2 (U83461), Transferrin (S95936), Metallothionein-I-A (K01383), Metallothionein IIa (V00594), Metallothionein III (M93311), Mitochondrial creatine kinase (CKMT) (J04469), Mitochondrial isocitrate dehydrogenase (NADP+) (X69433), Mitochondrial aspartate aminotransferase (M22632), Mitochondrial aldehyde dehydrogenase x (M63967), Aldehyde dehydrogenase type III (ALDHIII) (M74542). Aldehyde dehydrogenase 6 (U07919), Aldehyde dehydrogenase (ALDH8) (U37519), Aldehyde reductase (J04794), Oxidoreductase HHCMA56 (U13395), NAD+-isocitrate dehydrogenase (U87972), Selenoprotein P (Z11793), Salivary peroxidase (U39573), Glutathione S-transferase (GSTM5) (L02321), Glutathione S-transferase theta 2 (GSTT2) (L38503), Mitochondrial NADH-ubiquinone reductase 24 Kd subunit (M22538), Succinate dehydrogenase iron-protein subunit (sdhB) (U17886), Carnitine Calcium-Binding protein Mitochondrial (HG4749-HT5197), Heme oxygenase-2 (S34389), p38beta MAP kinase (U53442), C-Ki-Ras P21 (HG2036-HT2090), Protein kinase (MLK-3) (L32976), Kinase Myt1 (U56816), Anaplastic lymphoma kinase receptor (U66559), Receptor tyrosine kinase TrkC (U05012), G protein-coupled receptor kinase (GRK6) (L16862), G-protein coupled receptor (X95876), MEK kinase 3 (U78876), MNK1 (AB000409), JNK activating kinase (JNKK1) (U17743), FK506-binding protein (FKBP) (M34539), Insulin-stimulated protein kinase 1 (ISPK-1) (U08316), Serine Kinase Psk-H1 (HG1019-HT1019), Calcium, calmodulin-dependent protein kinase II gamma (U50360), Notch 4 (U89336), Myelin associated oligodendrocytic basic protein (D28114), Axin (AF009674), Neurofilament H (X15306), Neurofilament subunit NF-L (X05608), Neurofilament triplet L protein (U57341), Axonal transporter of synaptic vesicles (X90840), Cytokeratin 4 (X07695), Cytokeratin 17 (Z19574), K6b epidermal keratin type II (L00205), Dystroglycan (DAG1) (L19711), Macrophage capping protein (M94345), LAG-1 (X53683), Keratin type 11 (58 kD) (M21389), Skeletal muscle alpha 2 actinin (M86406), 22 kDa smooth muscle protein (SM22) (M95787), Myf-4 (X17651), and MYF6 (X5201 1), Non-muscle myosin light chain MLC (M22919), 815A9.1 myosin heavy chain (AF001548), Myosin-I beta (X98507), Fibroblast muscle-type tropomyosin (M12125), Cadherin FIB1 (AB000895), Cadherin FIB2 (AB000896), Contactin (Z21488), Fibronectin (FN precursor) (X02761), Beta-2 integrin alphaD (U40279), B-cam (X80026), Alpha-1 collagen type II (M60299), elastase IIB (M16653), Basement membrane heparan sulfate proteoglycan (X62515), Guanylate kinase (GUK1) (L76200), RAD23A homolog (AD000092), Mismatch repair protein (hMLH1) (AF001359), thymidylate kinase (CDC8) (L16991), Inhibitor of apoptosis protein 1 (U45878), Lysosome-associated membrane protein-2 (S79873), GM2 activator protein (X62078), alpha mannosidase (U37248), Alpha mannosidase II isozyme (L28821), Beta-galactoside alpha-23-sialyltransferase (SIAT4A) (L13972), Gal beta-13 GalNAc alpha-23 sialyltransferase (ST3Gal II) (U63090), Gal-beta(1-3/1-4)GlcNAc alpha-23-sialyltransferase (X74570), G9 encoding sialidase (X78687), Acid sphingomyelinase (ASM) (M59916), N-acetylglucosaminyltransferase I (GlcNAc-TI) (M55621), Beta-globin (U01317), Spot14 (Y08409), Spermidine synthase (M64231), Spermidine/Spermine N1-Acetyltransferase (HG172-HT3924), Prepro-plasma carboxypeptidase B (M75106), putative purinergic receptor P2Y10 (AF000545), Ubiquitin (M26880), Semaphorin V (U28369), aminoacylase 1 (L07548), N-acetylglucosaminyltransferase V (D17716), apolipoprotein apoC-IV (U32576), Alpha topoisomerase (L47276), Azurocidin (M96326), Uridine phosphorylase (X90858), Guanine nucleotide regulatory protein (tim1) (U02082), Guanylate binding protein isom II (GBP-2) (M55543), soluble guanylate cyclase (X66534), Osteomodulin (AB000114), Deoxyuridine nucleotidohydrolase (U31930), Calcium-sensing receptor (D50855), Granule membrane protein-140 (M25322), ELAM-1 ligand fucosyltransferase (ELFT) (M58597), p52 and p64 N-Shc (D84361), Crystallin Alpha A (HG3286-HT3463), Polypeptide 7B2 (Y00757), Tax helper protein 1 (D14827), Preferentially expressed antigen of melanoma (PRAME) (U65011), MNK1 (AB000409), UDP Galactose 4 epimerase (L41668), IPL (AF001294), Carbonyl reductase (J04056), Uromodulin (Tamm-Horsfall glycoprotein) (M15881), Enigma (L35240), 40871 sequence (U72507), Endogenous retroviral protease (M27826), lrp (X79882), Phosphomevalonate kinase (L77213), Unknown function protein (AF015910), dihydrodiol dehydrogenase (U05861), Modulator recognition factor I (MRF-1) (M62324), transglutaminase (M98447), Cpg-Enriched Dna Clone S19 (HG3995-HT4265), AML1b protein (D43968), Effector cell protease receptor-1 (EPR-1) (L32866), Folylpolyglutamate synthetase (M98045), Zn-alpha2-glycoprotein (X59766), Glucose transporter-like protein-III (GLUT3) (M20681), CO-029 (M35252), Pyruvate kinase type L (M15465), Ornithine transcarbamylase (K02100), Beta-casein (X13766), DCC (deleted in colorectal cancer) (S81294), Myeloid progenitor inhibitory factor-1 MPIF-1 (U85767), Carcinoembryonic antigen (CEA) (M29540), Estrogen sulfotransferase (U55764), Paraoxonase 2 (PON2) (L48513), TESK1 (D50863), Glucokinase (GCK) (M90299), Biphenyl hydrolase-related protein (X81372), Centrin (U03270), Insulin (J00268), Calcium activated neutral protease large subunit (muCANP) (X04366), Translational initiation factor (eIF-2) alpha subunit (J02645), Oncogene M11-Af4, Fusion Activated (HG4757-HT5207), PSE-binding factor PTF gamma subunit (U44754), TIMP-3 (D45917), Zinc finger protein ZNF133 (U09366), GP-39 cartilage protein (Y08374), Transmembrane 4 superfamily protein (SAS) (U01160), Gastrin-releasing peptide (K02054), Carnitine palmitoyltransferase I type I (Y08682), HIC-1 fragment (L41919), Diacylglycerol kinase (X62535), (clone NF 10) cytochrome P-450 nifedipine oxidase (J04449), MAGE-2 (L18920), (clone F-T03796) STM-2 (L43964), Chondroitin sulfate proteoglycan (MCSP) (X96753), Importin alpha 6 (AF005361), RNAse A (D26129), Peripherin (PRPH) (L14565), Treacher Collins syndrome (TCOF1) (U76366), Aminoimidazole carboxamide ribonucleotide transmylase/inosinicase (D82348), CGM7 nonspecific cross-reacting antigen (NCA) (D90276), G9a (X69838), CLA-1 (Z22555), Mevalonate pyrophosphate decarboxylase (MPD) (U49260), Acyl-CoA thioester hydrolase (U91316), Clathrin, Light Polypeptide B (HG2797-HT2906), Tissue plasminogen activator (PLAT) (K03021), Antigen of paraneoplastic sensory neuronopathy patients (M62843), Autoantigen NOR-90 (X56687), MAC30 (L19183), Dihydroorotate dehydrogenase (M94065), Neogenin (U61262), T-Plastin (HG2755-HT2862), Sarcolipin (SLN) (U96094), Fetal apolipoprotein AI precursor (X01038), Short-chain alcohol dehydrogenase family (D82061), 8-oxo-dGTPase (D16581), 4-aminobutyrate aminotransferase (S75578), RNase 4 (D37931), Glucose-6-phosphatase (U01120), ADP-ribosyltransferase (S74683), MHC Class I region proline rich protein (U63336), Achaete scute homologous protein (ASH1) (L08424), HFREP-1 (D14446), Ectodermal dysplasia protein (EDA) (U59228), Kruppel related zinc finger protein (HTF10) (L11672), DNA polymerase delta catalytic subunit (M80397), Elongin A (L47345), Succinate dehydrogenase iron-protein subunit (sdhB) (U17886), Ret Transforming (HG2825-HT2949), Proline-Rich protein Prb4 (HG4490-HT4876), S100A2 (Y07755), Nuclear factor erythroid 2 isom f (S77763), I-plastin (L20826), Gastrin releasing peptide receptor (GRPR) (M73481), Proto-onco Wnt7a (U53476), Farnesol receptor HRR-1 (U68233), P1-Cdc21(X74794), MSX-2 (D89377), secretin receptor (U28281), LDLC (Z34975), Methylenetetrahydrofolate Reductase (HG4234-HT4504), 6-pyruvoyl-tetrahydropterin synthase (D17400), ASM-like phosphodiesterase 3b (Y08134), Placental bikunin (U78095), Xanthine Dehydrogenase (HG3288-HT3465), Intestinal mucin (MUC2) (L21998), Secreted epithelial tumour mucin antigen (X52228), Profilin II (L10678), Fatty acids omega-hydroxylase (cytochrome P-450HKV) (D13705), HU-K4 (U60644), TCTEL1 (D50663), ITBA2 protein (X92896), PEP19 (PCP4) (U52969), Argininosuccinate synthetase (X01630), Expressed pseudo TCTA at t(1;3) translocation site (L41143), 6-phosphofructo-2-kinase/fructose-26-bisphosphatase (X52638), DROER homolog (D85758), Peroxisomal L-alanine:glyoxylate aminotransferase (X53414), X11 protein (U79255), PD-ECGF/TP (S72487), PBX2 (X59842), transcobalamin II (TCN2) (L02648), Palmitoylated erythrocyte membrane protein (MPP1) (M64925), Heat shock protein E coli DnaJ homolog (L08069), Int-1 mammary oncogene (X03072), Gravin (U81607), Glyoxalase II (X90999), and MURR1 (D85433), Alpha satellite and satellite 3 junction DNA sequence (M21305), ORF (M68864), Pregnancy-specific beta-1-glycoprotein PSGGA (M37755), LIMK-2 (D45906), A33 antigen precursor (U79725), Neuroendocrine-dlg (NE-dlg) (U49089), Nucleolar protein HNP36 (X86681), Mucin 3, Intestinal (HG2147-HT2217), B-cell pseudoautosomal boundary-like sequence (D55638), ELAM-1 ligand fucosyltransferase (ELFT) (M58597), Factor VII serine protease precursor (M13232), EPC-1 (U57450), 17 beta hydroxysteroid dehydrogenase type 2 (L 11708), Ini1 (U04847), Nup88 (Y08613), Cysteine protease CPP32 isom alpha (U13737), Int-2 protooncogene (X14445), GRB-7 SH2 domain (D43772), SH3 binding RES4-23A (AB000462), Platelet glycoprotein IIb (GPIIb) (M34344), R2 inducible membrane protein (X53795), MUC6 (HG880-HT880), RING protein (Y07829), EYA3 (Y10262), Prostasin (L41351), Lambda/iota-prot kinase C-interacting protein (U32581), EYA1 (Y10260), BENE (U17077), Prolargin (PRELP) (U41344), HRX-like protein (Y08836), Alpha-1-Antitrypsin (HG3517-HT3711), and ATP-binding cassette protein (U18237).

23. The method of claims 20, or 21, wherein the at least one gene product further comprises KIAA0176 (D79998), KIAA0123 (D50913), KIAA0320 (AB002318), KLAA0198 (D83784), KIAA0163 (D79985) and KLAA0246 (D87433), KIAA0028 (D21851), KIAA0224 (D86977), KIAA0018 (D13643), KIAA0113 (D30755), KIAA0011 (D13636), KIAA0181 (D80003), KIAA0182 (D80004), and KIAA0317 (AB002315), KIAA0159 (D63880), KIAA0384 (AB002382), KIAA0207 (D86962), KIAA0175 (D79997), KIAA0049 (D30756), KIAA0061 (D31765), KIAA0154 (D63876), KIAA0109 (D63475), KIAA0057 (D31762), KIAA0186 (D80008), KIAA0006 (D13631), KIAA0095 (D42085), KIAA0192 (D83783), KIAA0199 (D83782), KIAA0263 (D87452), KIAA0140 (D50930), KIAA0048 (D28588), KIAA0040 (D25539), KIAA0008 (D13633), KIAA0030 (D21063), KIAA0051 (D29640), KIAA0153 (D63487), KIAA0144 (D63478), KIAA9001 (D42040), KIAA0107 (D14663), KIAA0118 (D42087), KIAA0130 (D50920), and KIAA0167 (D79989).

24. The method of claim 1, further comprising modulating the level of a gene product in Tables 16, 17 or 18, or CD18.

25. A method for treating or preventing multiple sclerosis (MS) comprising administering to a subject with MS a composition that causes a decrease in the activity level or expression of a gene product selected from the group consisting of those genes indicated by a plus (+) sign in Tables 1-15, other than those indicated by an asterisk.

26. The method of claim 25, further comprising a second MS therapy.

27. The method of claim 26, wherein the second MS therapy is given before the composition.

28. The method of claim 26, wherein the second MS therapy is given after the composition.

29. The method of claim 26, wherein the second MS therapy is given concurrent with the composition.

30. The method of claim 26, wherein said second MS therapy is interferon β1a, interferon β1b, glatiramer acetate, and mitoxantrone.

31. The method of claim 25, wherein the composition comprises peptide.

32. The method of claim 25, wherein the composition comprises a small molecule.

33. The method of claim 25, wherein the composition is an organo-pharmaceutical.

34. The method of claim 25, wherein the composition comprises an expression cassette comprising a nucleic acid encoding an antisense construct or a ribozyme targeting the selected gene product, and a promoter active in eukaryotic cells, said nucleic acid operably linked to said promoter.

35. The method of claim 34, wherein said nucleic acid is comprised in a non-viral vector.

36. The method of claim 34, wherein said nucleic acid is comprised in a viral vector.

37. The method of claim 36, wherein said viral vector is an adenoviral vector, an adeno-associated viral vector, a retroviral vector, a herpesviral vector, a vaccinia viral vector or a polyoma viral vector.

38. The method of claim 34, wherein said promoter is a constitutive promoter, a tissue specific promoter or an inducible promoter.

39. The method of claim 38 wherein the tissue specific promoter is a neuronal cell promoter, a glial cell promoter, a monocyte promoter, a lymphocyte promoter, or a B cell promoter.

40. The method of claim 34, wherein said expression cassette further comprises a polyadenylation signal.

41. The method of claim 25, wherein said composition is administered intradermally, subcutaneously, intramuscularly, intraperitoneally, intravenously, intranasally, intraalveolarly, parenterally, intrathecally, intraparenchymally or intraperitoneally.

42. The method of claim 25, wherein said composition is administered to said mammal more than once.

43. The method of claim 25, wherein said composition is administered to said mammal in repeated discrete dosings.

44. The method of claim 25, where the at least one gene product comprises autoantigen calreticulin (M84739), ubiquitin-conjugating enzyme (UBE2I) (U45328), Wiskott-Aldrich syndrome protein (WASP) (U12707), placental type alkaline phosphatase (ALP-1) (J04948), and extracellular-superoxide dismutase (SOD3) (J02947).

45. The method of claim 25 or 44, where the at least one gene product comprises Protein tyrosine phosphatase sigma (U35234), expressed pseudo T-cell leukemia translocation-associated (TCTA) (L41143), Alpha1(XI) collagen (COL11A1) (U12139), Tyk2 non-receptor protein tyrosine kinase (X54637), and Spliceosomal protein Sap 62 (HG3033-HT3194).

46. The method of claim 25 or 44, where the at least one gene product comprises anti-B cell autoantibody IgM heavy chain variable VDJ region (U24683), Ig Heavy Chain, Vdjrc Regions (HG3731-HT4001), Ig-related 14.1 protein (M27749), Omega light chain protein 14.1 (Ig lambda chain related) (M34516), IGHA1 from Ig germline H-chain G-E-A region A: gamma-3 5 (J00220), Fc-gamma-RIIA IgG Fc receptor class IIA (X68090), Fc Receptor Iib3 For Igg, Low Affinity (HG491-HT491), Ig-like transcript 2 (U82279), Ig Heavy Chain Vdjc Regions (HG4458-HT4727), Ig J chain (M12759), and Ig alpha 2=Ig A heavy chain allotype 2; Also: S55735 (S71043).

47. The method of claim 44, 45, or 46, wherein the at least one gene product further comprises NRAMP1 (D50402), Leukocyte adhesion protein (LFA-1/Mac-1) (M15395), T cell receptor zeta-chain (J04132), T-cell receptor T3 gamma polypeptide (X04145), Interferon (IFN-alpha-M1) (M27318), Interferon alpha IFN-alpha 6 (X02958), Alpha interferon (V00551), Interferon-gamma receptor alpha chain (U19247), Interferon gamma receptor accessory factor-1 (AF-1) (U05875), TNF receptor (M58286), Tumor Necrosis Factor Receptor 2 Associated protein Trap3 (HG4683-HT5108), IFNB 1 (V00535), IL-4 splice variant (X81851), Interleukin-6-receptor (X58298), Interleukin-8 receptor type B (IL8RB)/U11877 (U11877), Interleukin-15 receptor alpha chain precursor (IL15RA) (U31628), Complement component 2 (C2) allele b (L09708), Complement component C9 (X02176), Adipsin/complement factor D (M84526), Complement component properdin; Also: X57748 (M83652), C5a anaphylatoxin receptor (M62505), Lipocortin (X05908), Beta-hexosaminidase beta-subunit (HEXB) (M23294), L-histidine decarboxylase (D16583), GPSAT=glycophorin SAT; Also: L31860 (S77893), Tyrosine Kinase Receptor Axl 2 (HG162-HT3165), Phospholipid Transfer protein (HG3945-HT4215), MHC class I-related protein; Also: X91625, U65416, X92841 (L14848), MHC class II HLA-DP light chain (M57466), MHC class II HLA-DR-beta-1*09012 (HLA-DRB1*09012) (M96132), MBP-2 MHC binding protein 2 (X65644), Platelet/endothelial cell adhesion molecule-1 (PECAM-1) (L34657), ME491/CD63 antigen (X62654), Heat shock protein hsp40 homolog (U40992), Heat-shock protein HSP70B′ (X51757), Thromboxane synthase (M80647), Thromboxane A2 receptor (D38081), Thrombospondin 2 (HG896-HT896), Granulocyte colony-stimulating factor receptor (CSF3R) (M59820), Plasminogen activator inhibitor type 1 N-terminus (X04729), Autoimmune Antigen Thyroid Disease-Related Antigen (HG3578-HT3781), Integrin beta-5 subunit (X53002), Integrin beta 7 subunit (S80335), Neuronal PAS1 (NPAS1) (U77968), Prointerleukin 1 beta (X04500), Interleukin 1 receptor antagonist IRAP (X53296), R kappa B (U08191), Cathepsin C (X87212), Lymphocyte Antigen Hla-G3 (HG273-HT273), Lymph node homing receptor (M25280), Monocyte chemoattractant protein-4 precursor (MCP-4) (U46767), thymosin beta (D82345), Tissue inhibitor of metalloproteinase 4 (U76456), Pancreatic phospholipase A-2 (PLA-2) (M21056), Fetal brain adenylyl cyclase (L05500), Adenylyl cyclase (L21993), Guanine nucleotide-binding protein (Gi) alpha subunit (M27543), Type 2 inositol 1 4 5-trisphosphate receptor (D26350), Putative G protein-coupled receptor (AZ3B); Also:U62027 (U28488), GNAT1 transducin alpha-chain (X15088), Transducin beta-2 subunit (M36429), Transducin-like enhancer protein (TLE3) (M99438), Low-Mr GTP-binding protein (RAB31) (U59877), 43 kDa inositol polyphosphate 5-phosphatase (Z31695), RAB7 (X93499), Ras Inhibitor Inf (HG511-HT511), R-ras (M14949), RasGTPase activating protein (D78156), Clone 110298 (L43579), Rab GDI (D13988), RhoE=26 kda GTPase homolog (S82240), HSPDE4C1 3 ,5-cyclic AMP phosphodiesterase (Z46632), SLP-76 associated protein (U93049), Calmodulin (M19311), Calmodulin dependent phosphodiesterase PDE1B1 (U56976), Calcineurin A catalytic subunit (S46622), Cyclophilin C (S71018), Cyclophilin-like protein (U37221), Secreted cyclophilin-like protein (SCYLP) (M63573), Fk506-Binding protein (HG1139-HT4910), Phospholipase C (M37238), GTPase activating protein (rap1GAP) (M64788), RasGTPase activating protein (D78156), Ras-Specific Guanine Nucleotide-Releasing Factor (HG2510-HT2606), Ras-related protein Rab5b (X54871), Ras inhibitor (Rin1) (L36463), RIN protein (Y07565), Guanine Nucleotide-Binding protein Rap2 (HG1996-HT2044), Guanine nucleotide-binding protein (Gi) alpha subunit (M27543), Guanine nucleotide-binding protein G-s-alpha-3 (M21142), Small G protein (Gx) (M64595), pp52=B lymphocyte signal transduction (S58733), GPR3 G protein-coupled receptor (U18550), Dishevelled homolog (DVL) (U46461), Protein kinase C delta-type (D10495), ERK6 extracellular signal regulated kinase (X79483), Tyrosine kinase (TXK) (L27071), Serine/Threonine Kinase (HG2709-HT2805), Tyrosine kinase (ELK1) (M25269), Casein kinase I gamma 2 (U89896), Calcium, calmodulin-dependent protein kinase II gamma (U50360), ERK6 extracellular signal regulated kinase (X79483), Serine/threonine kinase MNB (mnb) (U52373), T cell-specific tyrosine kinase (L10717), (clone FBK III 11c) protein-tyrosine kinase (DRT) (L41939), Phosphorylase kinase (PSK-C3) (M31606), p35 regulatory subunit of cdk5 kinase (X80343), 40 kDa protein kinase related to rat ERK2 (Z11695), PCTAIRE-3 serine/threonine protein kinase (X66362), Protein-tyrosine-phosphatase D1 (X79510), Tyrosine Phosphatase 1; Also: U12128 (HG3187-HT3366), Protein tyrosine phosphatase delta (L38929), Protein phosphatase 2A alpha subunit (M64929), Fructose 6-phosphate 2-kinase/fructose 2 6-bisphosphatase (D49817 and D49818), Inositol polyphosphate 1-phosphatase (L08488), Protein phosphatase 5 (X89416), Protein phosphatase X (X70218), InsP3 5-phosphatase; Also: Z31695 (X77567), Putative G protein-coupled receptor (AZ3B) (U28488), Cyclic nucleotide phosphodiesterase PDE2A3 (U67733), RGS3 (AF006609), Beta catenin/TCF-4 (Y11306), Nicotinic acetylcholine receptor alpha4 subunit precursor (U62433), Alpha-A1-adrenergic receptor (M76446), Beta 4 nicotinic acetylcholine receptor subunit (U48861), m3 muscarinic acetylcholine receptor (CHRM3) (U29589), Olfactory receptor cluster (U58675), Vasopressin V1b receptor (D31833), 43 kD acetylcholine receptor-associated protein (Rapsyn) (Z33905), Atrial natriuretic factor (M54951), Monoamine oxidase A (MAOA) (M68840), Transcobalamin I (J05068), Cholecystokinin type A receptor (CCK-A) (U23430), Growth hormone-releasing hormone receptor (L01406), Follicle stimulating hormone receptor (M65085), Chorionic gonadotropin beta subunit (K03189), CB2 (peripheral) cannabinoid receptor (X74328), Beta-2-adrenergic receptor (M15169), Spasmolytic polypeptide (SP) (X51698), Thyroid transcript factor 1 (X82850), Platelet factor 4 (PF4) (M25897), Preproinsulin; Also: M10039 (V00565), Insulin-like growth factor binding protein 5 (IGFBP-5) (M65062), IGF binding protein complex acid-labile subunit a (M86826), Glucagon-like peptide-1 receptor with CA dinucleotide repeat (U01157), Choline Acetyltransferase (HG4051-HT4321), hIRS-1=rat insulin receptor substrate-1 homolog (S85963), NMDA receptor modulatory subunit 2A (hNR2A) (U09002), Metabotropic glutamate receptor 4; Also: X80818 (U92457), Excitatory amino acid transporter 4 (U18244), PephBGT-1 betaine-GABA transporter (U27699), Corticotropin releasing factor receptor (L23333), Parathyroid hormone-related protein (M17183), Somatostatin receptor subtype 3 (SSTR3); Also: Z86000 (M96738), T3 receptor-associating cofactor-1; Also: U37146 (S83390), Telencephalin precursor (U72671), (clone CR-3) teratocarcinoma-derived growth factor 3 TDGF3 (M96956), P2× purinoceptor (AF000234), P2X7 receptor (Y09561), Group-specific component vitamin D-binding protein (M11321), Thymidylate kinase (CDC8) (L16991), DNA-repair protein (XRCC1) (M36089), Replication factor C 37-kDa subunit (M87339), Sox3 (X71135), SOX-4 protein (X70683), SOX5=Sry-related HMG box (S83308), RAD51 (D14134), Mutator (hMSH2) (U03911), Cdc25A (M81933), RBP2=retinoblastoma binding protein 2 (S66431), SWI/SNF complex 60 KDa subunit (BAF60c) (U66619), Retinoic acid receptor gamma 1 (M38258), Cellular retinoic acid-binding protein (S74445), Cyclin I (D50310), Checkpoint suppressor 1 (U68723), Cell cycle checkpoint control protein (U53174), PRAD1 cyclin (X59798), Cyclin F (Z36714), CCG1/TFIIDp250 (D90359), (clone mf18) RNA polymerase II (L37127), TFIIIC Box B-binding subunit (U02619), Ets domain protein ERF (U15655), Myelin TRANSCRIPTION FACTORS 1 (MTF1) (M96980), Paired Box Hup1 (HG2188-HT2258), Homeotic protein Hox54 (HG3502-HT3696), Homeobox (clone HHO.c13); Also: X17360 (X04706), HOX 5.1 protein (X17360), Homeobox protein (PHOX1) (M95929), Homeotic protein Hpx-42 (HG3884-HT4154), Khead Family Afx1 (HG4245-HT4515), AFX protein (X93996), PAX3B=transcription factor; Also: S69369 (S69370), Pur (pur-alpha) (M96684), HOX7 (M76732), Delta opioid receptor (U07882), Nuclear orphan receptor LXR-alpha (U22662), PAX3A=TRANSCRIPTION FACTOR (S69369), Basic Transcription Factor 44 Kda Subunit (HG3748-HT4018), B-cell specific TRANSCRIPTION FACTOR (BSAP) (M96944), Nucleolar autoantigen No 55 (U47621), Transcription elongation factor (SII) (M81601), Polyadenylate binding protein II (Z48501), ERF-2 (X78992), DNF1552 (lung) (J03068), High mobility group protein HMG-I(Y) (L17131), Putative HMG-17 non-histone protein (X13546), B-myb (X13293), Erythroid-specific TRANSCRIPTION FACTORS EKLF (U65404), Faciogenital dysplasia (FGD1) (U11690), Br140(M91585), MSS1 (D11094), Nuclear factor I-X (L31881), Zn-15 related zinc finger protein (rlf) (U22377), Id-related helix-loop-helix protein Id4 (U28368), DNA-binding protein (HRC1) (M91083), DNA binding protein (HPF2) (M27878), Indian hedgehog protein (IHH) (L38517), PBX2 (X59842), Transcription Factor Btf3 Homolog M90355 (HG4518-HT4921), Proto-Oncogene C-Myc (HG3523-HT4899), Spi-1 proto-oncogene (X52056), Spi-B (X66079), Proliferating cell nuclear antigen (PCNA) promoter region (J05614), p50-NF-kappa B homolog (S76638), Basic TRANSCRIPTION FACTOR 62 kD subunit (BTF2) (M95809), Basic Transcription Factor 44 Kda Subunit (HG3748-HT4018), TRANSCRIPTION FACTOR SIM2 short form (U80457), C-kit proto-oncogene (X06182), Id-related helix-loop-helix protein Id4 (U28368), C/EBP gamma (U20240), Interleukin-1 beta convertase (IL1BCE, CASPASE 1) (M87507), DNA (cytosin-5)-methyltransferase (X63692), RNA polymerase subunit hRPB 33 (J05448), Inducible poly(A)-binding protein (U33818), H12 histone H1 (X57129), H2B/h/Z80780 (Z80780), H4/h H4 histone (X60487), Myeloid elf-1 like factor (MEF) (U32645), TRANSCRIPTION FACTOR GATA-2 (M77810), NBK apoptotic inducer protein (X89986), L-type calcium channel (Z26256), CNG2=cyclic nucleotide-gated cation channel/S76067 (S76067), Vacuolar H+-ATPase (L35249), K+ channel beta 2 subunit (U33429), Epithelial amiloride-sensitive sodium channel gamma (X87160), Channel-like integral membrane protein (CHIP28) (M77829), K-C1 cotransporter (hKCC1) (U55054), Voltage-gated calcium channel beta subunit (U07139), DHP-sensitive calcium channel gamma subunit (CACNLG) (L07738), P/Q-type calcium channel alpha1 subunit; Also: U79663 (X99897), Acid sphingomyelinase (ASM) (M59916), N-acetylglucosaminyltransferase I (GlcNAc-TI) (M55621), Alpha (1,3) fucosyltransferase (FUT6), GalNAc-T4/Y08564 (Y08564), Acid ceramidase (U70063), Beta-galactoside alpha26-sialyltransferase (SIAT1)/U67849 (U67849), UCP1 uncoupling protein (X51954), UCP3S (U82818), Glutathione transferase M3 (GSTM3) (J05459), Selenium-binding protein (hSBP)/U29091 (U29091), Manganese superoxide dismutase SOD2 (X07834), ATL-derived factor/thioredoxin (X77584), Ceruloplasmin (ferroxidase) (M13699), FDXR (adrenodoxin reductase); Also: HG2836-HT2962 (M58509), Mitochondrial NAD(P)+ dependent malic enzyme (M55905), HALPHA44 alpha-tubulin (X06956), Alpha1(XI) collagen (COL11A1) (U12139), Alpha-1 collagen type II s 1 2 and 3 (M60299), Skeletal muscle LIM-prot SLIM2 (U60116), Beta-myosin heavy chain (M21665), Dermal fibroblast elastin (X52896), Cadherin (D88799), Cardiac troponin I (X90780), Cardiac troponin T (X74819), Chitotriosidase precursor (U29615), Apolipoprotein AII (X04898), Hair keratin hHb5 (X99140), Hair keratin hHb6 (X99142), High-sulphur keratin (X63755), Collagen Type Viii Alpha 1 (HG2614-HT2710), a1(XIX) collagen chain; Also: U09279 (D38163), Myotubularin related protein 1 (MTMR1) (U58032), Myosin VIIA (USH1B) (U39226), Beta-myosin heavy chain; Also: X52889 (M21665), Myosin, Heavy Polypeptide 10, Non-Muscle (HG2175-HT2245), Myosin; Also: M36769 (Z38133), Smooth muscle myosin heavy chain isoform Smemb (S67247), Embryonic/atrial myosin light chain (MLC-1-emb/A isoform) (M37075), High-sulphur keratin (X63755), Microtubule-associated protein 2 (MAP2) (U01828), Microtubule-associated tau protein (X14474), Alpha 1 syntrophin (S81737), Dystrobrevin-epsilon; Also: U46744 (U46746), Dynactin (U73799), Dynein HeavyChain (HG2417-HT2513), Enteric smooth muscle gamma-actin (D00654), Non-muscle alpha-actinin (M95178), Microtubule-associated protein 2 (MAP2) (U01828), Duchenne Muscular Dystrophy protein (Dmd); Also: M18533 (HG2260-HT2349), Synaptophysin (p38) (X06389), Ladinin (LAD) (U42408), Beta B1-crystallin (U35340), Cylicin (Z22780), Cylicin II (Z46788), Loricrin (M94077), Intestine-specific annexin (Z11502), Annexin V (ANX5) (U01691), Phospholipid scramblase (AF008445), BAK BCl-2 homolog (X84213), Bcl-2 binding component 3 (bbc3) (U82987), Survival motor neuron protein (U80017), Phosphoribosylpyrophosphate synthetase (X15331), Uroporphyrinogen III synthase (J03824), Muscle glycogen synthase (J04501), ATP synthase B chain (D28383), Deoxyhypusine synthase (U26266), Pyrroline 5-carboxylate synthetase (X94453), S-adenosylmethionine synthetase (D49357), G7a valyl-tRNA synthetase (X59303), Aromatase (estrogen synthetase) (X13589), PP15 (placental protein 15) (X07315), Placental protein 5 (D29992), Delta3, delta2-CoA-isomerase (L24774), Topoisomerase type II (M27504), Thyroid Peroxidase (HG2999-HT4756 or M25715), Triglyceride lipase (M29194), Hormone-sensitive lipase testicular isoform (U40002), LDL-phospholipase A2 (U24577), Carbonic anhydrase VII (CA VII) (M76424), Procarboxypeptidase A1 (X67318), Prepro-plasma carboxypeptidase B (M75106), Carboxypeptidase M (J04970), Melanocortin 5 receptor (MC5R) (L27080), PSG10 pregnancy specific glycoprotein 10 (X17098), Glutamate pyruvate transaminase (GPT) (U70732), Neurotensin receptor (X70070), hnRNP-E2 (X78136), Small Nuclear Ribonucleoprotein U1, 1snrp (HG4557-HT4962), Heterologous ribonucleoprotein A0 (U23803), Ribosomal protein L3 (X73460), XIST a (X56199), Sel-1 like (U11037), p126 (ST5) (U15131), Cleavage signal 1 protein (M61199), Membrane glycoprotein M6 (D49958), Post-synaptic density protein 95 (PSD95) (U83192), LDL-receptor related protein (X13916), Clone lambda 5 semaphorin (U33920), major transcript I (U27333), A9A2BRB7 (CAC)n/(GTG)n repeat-containing clone (U00952), Activin beta-A subunit (2) (X57579), Adducin Alpha Subunit 2 (HG651-HT4201), Gps2 (GPS2) (U28963), Involucrin (M13903), Docking protein (signal recognition particle receptor) (X06272), Tight junction (zonula occludens) protein ZO-1 (L14837), Apomucin (Z48314), Pancreatic mucin (J05582), Mucin/M22406 (HG1067-HT1067), Z68155 and others (X79683), Histo-blood group ABO protein (U15197), Hs-cul-4A (U58090), Plectin (PLEC1) (U53204), TPRC (X99720), Prolyl 4-hydroxylase alpha subunit (M24486), Prolyl 4-hydoxylase beta subunit (X05130), 4F2 glycosylated heavy chain (4F2HC) antigen (M21904), Cosmid clone LUCA17/3p213 (AC002077) clone 1D2 (Z78289), Uridine nucleotide receptor (UNR) (U40223), Pre-splicing factor SRp20(D28423), Lfp35 from BRCA1, Rho7 and vatI (L78833), p97 homolog (D85939), HIV-1 Nef interacting protein (Nip7-1) (U83843), HFH4 (X99350), XG (clone RACE5) (Z48519), LLGL (D50550), MUF1 protein (X86018), Unknown protein (clone ICRFp507O0882) (Z70220), Heregulin-beta2 (M94167), NB-1 (M58026), Caveolin (Z18951), ATP-binding cassette protein (ABC2) (U18235), SNC19 sequence (U20428), UDP-galactose transporter 2 (D88146), CE29 4.1 (CAC)n/(GTG)n repeat-containing clone (U00928), gp25L2 protein (X90872), E6-AP ubiquitin protein ligase 3A (AF002224), Ribosomal DNA repeating unit (U13369), Vacuolar proton ATPase subunit D (X71490), Inter-alpha-trypsin inhibitor subunit 3 (X16260), Leukemia virus receptor 1 (GLVR1) (L20859), Clone S171 (L40393), Clone cD24-1 Huntington's candidate region fragment (L37199), FLII (U80184), Inhibin A-subunit (M13981), K12 protein precursor (U77643), Factor X (blood coagulation factor) (L29433), M25296 and others (M31776), Chondroadherin (U96769), Clone 23933 sequence (U79273), Protein immuno-reactive with anti-PTH polyclonal antibodies (U28831), Metastasis-associated mta1 (U35113), Bleomycin hydrolase (X92106), Gu binding protein (U78524), 65 kDa hydrophobic protein (U17566), Ribosomal protein L18a Homolog (HG4390-HT4660), Terminal transferase (M 11722), Skeletal muscle LIM-protein SLIM2 (U60116), EVX1 (X60655), HNSPC (D82346), (23k/3) ubiquitin-conjugating enzyme UbcH2 (Z29331), Mg81 (HG909-HT909), Growth-arrest-specific protein (gas) (L13720), Peripheral myelin protein-22 (PMP22) 1B (U08096), Mouse transaldolase (U67611), hHKb1 protein (X81420), L-arginine:glycine amidinotransferase (X86401), Furin (X17094), UGT2BIO udp glucuronosyltransferase (X63359), Transmembrane protein Tmp21-IIex (X97444), U1 small nuclear RNP-specific C protein (X12517), Nicotinamide nucleotide transhydrogenase (U40490), Nicotinamide N-methyltransferase 1 and 5 ing region (U51010), Chymotrypsinogen (M24400), Netrin-2 like protein (NTN21) (U86759), M95971 (J05252), Phosphoprotein Tal2 (HG4068-HT4338), PAC clone DJ525N14/Xq23 (AC002086), 62 kDa paraneoplastic antigen (L02867), Transglutaminase E3 (TGASE3) (L10386), NB Neurofibromin (D12625), HIV-1 Nef interacting prot (Nip7-1) (U83843), Uterus ficolin-1 (D83920), NF-AT3 (L41066), WD protein IR10 (U57057), Beta-microseminoprotein (MSP) (M34376), Clk2 (L29218), TNNT2 11/X98482 (X98482), RR2 small subunit ribonucleotide reductase (X59618), DD96 (U21049), AP-3 complex beta3A subunit (U91931), Hemopexin (M36803), Recoverin (S62028), Cartilage matrix protein (CMP) (M55682), B56-delta (L76702), (˜95%) SFTPA2D (HG3928-HT4198), Antileukoprotease (ALP) from cervix uterus (X04470), CD171 protein (Y10207), Thyroid receptor interactor (TRIP9) (L40407), H19 RNA (spliced in silico) (M32053), (clone S171) (L40393), Unknown protein (U82306), Coronin (X89109), KIAA0055 (D29956), Telomerase reverse transcriptase (AF015950), Tigger 1 transposable element (U49973), Transcript ch132/S77361 (S77361), Low density lipoprotein receptor (M28219), Chondroitin/dermatan sulfate proteoglycan (PG40) core protein (M14219), Endometrial bleeding associated factor (U81523), Fra-2 (X16706), Phosphoglucomutase 1 (HG3893-HT4163), Steroid 11-beta-hydroxylase (CYP11B1) (M32879), RSRFR2 (X63380), Small proline rich protein (sprII), clone 174N (M21302), CHD5 protein (Y12478), Na+,K+-ATPase catalytic subunit alpha-III isoform (M37457), DP-2 (L40386), Propionyl-CoA carboxylase beta-subunit (M31169), NECDIN related protein (U35139), DNL1L from chromosome X region (L44140), GOS2 (M72885), Clones 23920 and 23921 sequence (U79271), Germline oligomeric matrix protein (COMP) (L32137), LISCH7 (liver-specific bHLH-Zip transcription factor) (AD000684), Cpg-Enriched Dna, Clone E18 (HG3991-HT4261), MADER (X70991), Bone morphogenetic protein-3b (D49493), Macrophage lectin 2 (D50532), Hereditary multiple exostosis (U96629), Erythrocyte membrane protein band 42 (EPB42) (M60298), Zinc finger protein (clone 647) (X16282), Branched chain decarboxylase alpha subunit (Z14093), Sterol regulatory element binding protein-2 (U02031), UDP-galactose translocator (D84454), Hermansky-Pudlak syndrome protein (HPS) (U65676), Ribonuclease k6 precursor/U64998 (U64998), Chromosome 12p15 BAC clone CIT987SK-99D8 sequence (U91327), Chromosome 17q12-21 clone pOV-2 (U18919), (clone p5-23-3) (L48692), Hypothetical protein Npiiy20/M76676 (HG167-HT167), Plakophilin; Also: X79293 (Z34974), 75-kD autoantigen (PM-Sc1) (M58460), High-mobility group phosphoprotein isoform I-C (HMGIC) (U28749), Alpha-1-antitrypsin-related protein (M19684), DLX-2 (Dlx2) (U51003), Blood platelet membrane glycoprotein Ib-alpha (GPIB) (M22403), Tumor-associated membrane protein homolog (TMP) (U43916), Clone A9A2BRB5 (CAC)n/(GTG)n repeat-containing (U00946), Zinc finger protein 45 (ZNF45) (L75847), Beige-like protein (BGL) (M83822), Splicing Factor Sc35 m 3 (HG3088-HT3263), HZF2 zinc finger protein (X78925), Cytosolic serine hydroxymethyltransferase (SHMT) (L11931), S-lac lectin L-14-II (LGALS2) (M87860), Angiotensin II type 2 receptor (U20860), MN1 protein (clone ICRFp507I0498); Also: X82209 (Z70218), Splicing factor, SF1-Bo isoform; Also: L49380 (Y08766), Pregnancy zone protein (X54380), Nuclear respiratory factor 1 (NRF1) (U44848), Clones MDP4 MDP7 microsomal dipeptidase (MDP) (J05257), Gastrin-binding protein/X98225 (X98225), Acrosin (EC 342110) (Y00970), Peptide Yy; Also: D13897_rna2 (HG2348-HT2444), G protein-coupled receptor (STRL22) (U68031), CGM1 (HG1728-HT1734), Cystatin D (HG1098-HT1098), (clone 353) DRAL (L42176), (clone CTG-B37) sequence (L10377), Down syndrome critical region 1 (DSCR1) alternative 1 (U85265), Zinc Finger protein Zfp-36 (HG3491-HT3685), EAR-1r (D16815), (clone s22i71) (L40396), NCBP interacting protein 1 (D59253), Erythroblastosis virus onco homolog 2 (ets-2) (J04102), BLR1 Burkitt's lymphoma receptor 1 (X68149), Trabecular meshwork-induced glucocorticoid response protein (AF001620), bHLH-PAS protein Jap3 (U60415), Guanylin (M97496), Dioxin-responsive (S81578), RD/X99296 (X99296), Plasma inter-alpha-trypsin inhibitor heavy chain H(3) (X14690), Major Yo paraneoplastic antigen (CDR2) (M63256), PCI (plasminogen activator inhibitor 3) from protein C inhibitor (M68516), KNP-Ib; Also: U53003 (D86062), MJD1=MJD1 protein {CAG repeats} (S75313), POM121-like 1 (D87002), Cell surface glycoprotein P3.58 (M55024), Oviductal glycoprotein (U09550), Kallmann syndrome (KAL) (M97252), PACAP receptor (D17516), Retinal pigment epithelium-specific 61 kDa protein (RPE65) (U18991), Squamous cell carcinoma antigen=serine protease inhibitor (S66896), Clone 23948 sequence (U79293), Albumin, 3; Also: HG2841-HT2970, HG2841-HT2968 (HG2841-HT2969), Protein containing SH3 domain SH3GL2 (X99657), HK2 hexokinase II (Z46376), Ribosomal protein S6 kinase 2 (RPS6KA2) (L07597), Phosphoglycerate mutase, muscle-specific (PGAM-M) (J05073), IP prostacyclin receptor (D38128), Pan-2 (HG2604-HT2700), Anion Exchanger 3 Cardiac Isom (HG4128-HT4398), Tax1 binding protein (U25801), Hepatic nuclear factor 1-alpha (TCF-1-alpha)/U73499 (U73499), M-phase phosphoprotein mpp9 (X98258), Brain 4 (X82324), PTX3/X97748; Also: M31166 (X97748), Uroporphyrinogen decarboxylase (URO-D)/M60891 (M60891), pTR7 repetitive sequence/X15675 (X15675), Erythropoietin receptor (M60459), Sulfite oxidase (L31573), HM145 (D10925), PEBP2aC1 acute myeloid leukaemia (Z35278), Trpc2 transcript (possible pseudo) (X89067), P3 (X12458), CD20 receptor (S7) (X07203), Aromatic amino acid decarboxylase (ddc) (M76180), DNA sequence from Huntington's Disease Region (Z69923), BAC clone GS244B22/7q21-q22/AC002450 (AC002450), Cysteine protease (D55696), Semaphorin (CD100) (U60800), DNA on chromosome 22q 11.2-qter contains GSTT1-2 (Z84718), WD repeat protein HAN11/U94747 (U94747), X64467_rna1 and others (M13928), Syt V (X96783), ltk; Also: D16105 (X52213), JTV-1 (JTV-1) (U24169), Alpha-L-iduronidase (M95740), Sterol carrier protein-X/sterol carrier protein-2 (SCP-X/SCP-2) (U11313), Aspartyl(asparaginyl)beta-hydroxylase; Also: U03109 (S83325), Presenilin 1 (PS1; S182); Also: L76517 (L76528), DAP-1 (X76105), SS-A/Ro ribonucleoprot autoantigen 60 kd subunit (M25077), Endothelin-B receptor (D13168), Ca2+-dependent activator protein secretion (U36448), Placenta (Diff33) (U49188), Putative OSP like protein (U89916), Peroxisomal 70 kD membrane protein; Also:X83467_rnal (M81182), Carnitine palmitoyltransferase II precursor (CPT1) (U09646), Isolate JuSo MUC18 glycoprot (3 variant); Also: M28882 (M29277), Cytochrome P450 (CYP2A13) (U22028), Cytochrome P450c21(M17252), and MBP1; Also: X15422 (X15954).

48. The method of claims 20, or 21, wherein the at least one gene product further comprises: KIAA0006 (D13631), KIAA0010 (D13635), KIAA0053 (D29642), KIAA0060 (D31766), KIAA0064 (D31764), KIAA0066 (D31886), KIAA0079 (D38555), KIAA0086 (D42045), KIAA0091 (D42053), KIAA0092 (D42054), KIAA0100 (D43947), KIAA0133 (D50923), KIAA0150 KIAA0152 (D63486), (D63484), KIAA0187 (D80009), KIAA0201 (D86956), KIAA0217 (D86971), KLAA0230 (D86983), KIAA0235 (D87078), KIAA0240 (D87077), KIAA0260 (D87449), KIAA0281 (D87457), and KIAA0358 (AB002356).

49. The method of claim 25, further comprising modulating the level of a gene product in Tables 16, 17 or 18, or CD18.

50. A method of identifying a composition useful in the treatment or prevention of multiple sclerosis (MS) comprising:
(a) providing a cell that expresses one or more genes identified in Tables 1-15, other than those indicated by an asterisk;
(b) contacting said cell with a candidate substance; and
(c) assessing the expression of the one or more genes,
wherein modulation of the expression of the one or more genes identifies said candidate substances as a composition useful in the treatment or prevention of multiple sclerosis MS.

51. The method of claim 48, wherein said cell is of neuronal, glial, endothelial, intravascular, perivascular, or central nervous system-infiltrating immune cell origin.

52. The method of claim 49, wherein said neuronal cell is a neural progenitor or stem cell.

53. The method of claim 49, wherein said glial cell is an astrocyte, microglia cell or oligodendrocyte.

54. The method of claim 49, wherein said intravascular cell is a myocyte.

55. The method of claim 49, wherein said perivascular cell is a pericyte.

56. The method of claim 49, wherein said central nervous system-infiltrating immune cell is a lymphocyte, a monocyte, or a B cell.
